SEQUENCE LISTING (135 sequences)

SEQ ID NO: 1 - DNA, 49 bp, Synthetic construct
  source 1..49: mol_type = other DNA
  misc_feature 1..49: note = Nucleotide sequence for oligo containing 15bp annealing antigen barcode
ccttggcacccgagaattccannnnnnnnnnnnnnncccatataagaaa

SEQ ID NO: 2 - DNA, 15 bp, Synthetic construct
  source 1..15: mol_type = other DNA
  misc_feature 1..15: note = Antigen barcode for CA/09
gcagcgtataagtca

SEQ ID NO: 3 - DNA, 15 bp, Synthetic construct
  source 1..15: mol_type = other DNA
  misc_feature 1..15: note = Antigen barcode for RSC3
cagtaagttcgggac

SEQ ID NO: 4 - DNA, 15 bp, Synthetic construct
  source 1..15: mol_type = other DNA
  misc_feature 1..15: note = Antigen barcode for HT593.1
tgaccttcctctcct

SEQ ID NO: 5 - DNA, 15 bp, Synthetic construct
  source 1..15: mol_type = other DNA
  misc_feature 1..15: note = Antigen barcode for KNH1209.18
gacctcattgtgaat

SEQ ID NO: 6 - DNA, 15 bp, Synthetic construct
  source 1..15: mol_type = other DNA
  misc_feature 1..15: note = Antigen barcode for 5768.04
taactcagggcctat

SEQ ID NO: 7 - DNA, 15 bp, Synthetic construct
  source 1..15: mol_type = other DNA
  misc_feature 1..15: note = Antigen barcode for 286.36
cagatgatccaccat

SEQ ID NO: 8 - DNA, 15 bp, Synthetic construct
  source 1..15: mol_type = other DNA
  misc_feature 1..15: note = Antigen barcode for BG505 T332N
atcgtcgagagctag

SEQ ID NO: 9 - DNA, 23 bp, Synthetic construct
  source 1..23: mol_type = other DNA
  misc_feature 1..23: note = Outer Reverse primer (RP) 1
ggtaggtgtgcactccactggtc

SEQ ID NO: 10 - DNA, 25 bp, Synthetic construct
  source 1..25: mol_type = other DNA
  misc_feature 1..25: note = Outer RP 2
caccactgtggccttgttktcctgg

SEQ ID NO: 11 - DNA, 25 bp, Synthetic construct
  source 1..25: mol_type = other DNA
  misc_feature 1..25: note = Outer RP 3
caccactgtggccttgttttcgttg

SEQ ID NO: 12 - DNA, 23 bp, Synthetic construct
  source 1..23: mol_type = other DNA
  misc_feature 1..23: note = Outer RP4
gtgttgtccttgctgtcctgatc

SEQ ID NO: 13 - DNA, 22 bp, Synthetic construct
  source 1..22: mol_type = other DNA
  misc_feature 1..22: note = Inner RP 1
gctcagggaagtagcccttgac

SEQ ID NO: 14 - DNA, 28 bp, Synthetic construct
  source 1..28: mol_type = other DNA
  misc_feature 1..28: note = Inner RP 2
ctcctcactcgagggygggaayaggctg

SEQ ID NO: 15 - DNA, 23 bp, Synthetic construct
  source 1..23: mol_type = other DNA
  misc_feature 1..23: note = Inner RP 3
gttcagagccatccaccttccac

SEQ ID NO: 16 - DNA, 49 bp, Synthetic construct
  source 1..49: mol_type = other DNA
  misc_feature 1..49: note = Forward Primer (FP) 1
aatgatacggcgaccaccgagatctacactctttccctacacgacgctc

SEQ ID NO: 17 - DNA, 25 bp, Synthetic construct
  source 1..25: mol_type = other DNA
  misc_feature 1..25: note = Forward Primer (FP) 2
aatgatacggcgaccaccgagatct

SEQ ID NO: 18 - protein, 11 aa, Synthetic construct
  REGION 1..11: note = Amino acid sequence for CDRH3- VH3-197-54
AKAPYGYEGDV

SEQ ID NO: 19 - protein, 20 aa, Synthetic construct
  REGION 1..20: note = Amino acid sequence for CDRH3- VH3-183-54
ARYSYYRYSDGTYYSSGFDV

SEQ ID NO: 20 - protein, 15 aa, Synthetic construct
  REGION 1..15: note = Amino acid sequence for CDRH3- VH3-80-54
ARVPFYGYHYGAPDV

SEQ ID NO: 21 - protein, 12 aa, Synthetic construct
  REGION 1..12: note = Amino acid sequence for CDRH3- VH3-167-75
TLTYYWSADFDV

SEQ ID NO: 22 - protein, 13 aa, Synthetic construct
  REGION 1..13: note = Amino acid sequence for CDRH3- VH1-58-54
ARGYYDDSFNFQY

SEQ ID NO: 23 - protein, 16 aa, Synthetic construct
  REGION 1..16: note = Amino acid sequence for CDRH3- VH3-114-75
ARDYSSGFFPRSGFQH

SEQ ID NO: 24 - protein, 11 aa, Synthetic construct
  REGION 1..11: note = Amino acid sequence for CDRH3- VH1-171-54
ARCGAYYDFDV

SEQ ID NO: 25 - protein, 14 aa, Synthetic construct
  REGION 1..14: note = Amino acid sequence for CDRH3- VH3-282-54
AAEYYSRDNYYEII

SEQ ID NO: 26 - protein, 6 aa, Synthetic construct
  REGION 1..6: note = Amino acid sequence for CDRH3- VH3-167-75
ATGLAV

SEQ ID NO: 27 - protein, 6 aa, Synthetic construct
  REGION 1..6: note = Amino acid sequence for CDRH3- VH3-167-75
ATGLAV

SEQ ID NO: 28 - protein, 12 aa, Synthetic construct
  REGION 1..12: note = Amino acid sequence for CDRH3- VH1-171-54
ARDYGNDWYFDV

SEQ ID NO: 29 - protein, 9 aa, Synthetic construct
  REGION 1..9: note = Amino acid sequence for CDRH3- VH1-151-54
ATLTTTGEY

SEQ ID NO: 30 - protein, 9 aa, Synthetic construct
  REGION 1..9: note = Amino acid sequence for CDRH3- VH1-151-54
TTLTTTGEY

SEQ ID NO: 31 - protein, 9 aa, Synthetic construct
  REGION 1..9: note = Amino acid sequence for CDRH3- VH1-151-54
TTLTTTGEY

SEQ ID NO: 32 - protein, 9 aa, Synthetic construct
  REGION 1..9: note = Amino acid sequence for CDRH3- VH1-151-54
TTLTTTGEY

SEQ ID NO: 33 - protein, 12 aa, Synthetic construct
  REGION 1..12: note = Amino acid sequence for CDRH3- VH3-197-54
AREEGVQVYFDV

SEQ ID NO: 34 - protein, 6 aa, Synthetic construct
  REGION 1..6: note = Amino acid sequence for CDRH3- VH3-167-75
TSGISQ

SEQ ID NO: 35 - protein, 12 aa, Synthetic construct
  REGION 1..12: note = Amino acid sequence for CDRH3- VH3-183-54
VREEGGQVYFDV

SEQ ID NO: 36 - protein, 12 aa, Synthetic construct
  REGION 1..12: note = Amino acid sequence for CDRH3- VH3-183-54
VREEGGQVYFDV

SEQ ID NO: 37 - protein, 12 aa, Synthetic construct
  REGION 1..12: note = Amino acid sequence for CDRH3- VH3-183-54
VREEGGQVYFDL

SEQ ID NO: 38 - protein, 12 aa, Synthetic construct
  REGION 1..12: note = Amino acid sequence for CDRH3- VH3-183-54
VREEGGQVYFDV

SEQ ID NO: 39 - protein, 9 aa, Synthetic construct
  REGION 1..9: note = Amino acid sequence for CDRH3- VH1-91-54
ARGMGYFDV

SEQ ID NO: 40 - protein, 9 aa, Synthetic construct
  REGION 1..9: note = Amino acid sequence for CDRH3- VH1-91-54
ARGMGYFDV

SEQ ID NO: 41 - protein, 9 aa, Synthetic construct
  REGION 1..9: note = Amino acid sequence for CDRH3- VH1-91-54
VRGMGYFDV

SEQ ID NO: 42 - protein, 9 aa, Synthetic construct
  REGION 1..9: note = Amino acid sequence for CDRH3- VH1-91-54
ARGGGYFDV

SEQ ID NO: 43 - protein, 9 aa, Synthetic construct
  REGION 1..9: note = Amino acid sequence for CDRH3- VH1-91-54
ARGMGYFDV

SEQ ID NO: 44 - protein, 13 aa, Synthetic construct
  REGION 1..13: note = Amino acid sequence for CDRH3- VH1-58-54
ARTYYDDAFTFQY

SEQ ID NO: 45 - protein, 13 aa, Synthetic construct
  REGION 1..13: note = Amino acid sequence for CDRH3- VH1-58-54
ARTYYDDAFTFQY

SEQ ID NO: 46 - protein, 11 aa, Synthetic construct
  REGION 1..11: note = Amino acid sequence for CDRH3- VH1-171-54
ARCAGYYDFDF

SEQ ID NO: 47 - protein, 16 aa, Synthetic construct
  REGION 1..16: note = Amino acid sequence for CDRH3- VH1-91-54
VLRGTRMVTIDPHFDV

SEQ ID NO: 48 - DNA, 363 bp, Synthetic construct
  source 1..363: mol_type = other DNA
  misc_feature 1..363: note = 112_22_HC Nucleotide sequence CDRH
caggtccagctccaggaatcaggtcccggccttgttaaaccttcacagtctttaagcatcacttgtatggtgagtggcttttccatcaccactaccaactactggtggcactggattcgacagccccccggcaagagcctggagtggctgggagaaattagatacgacgatgctactaagtacaatccctctctgaagtccagattaagtattactcgcgacaccggtaagaatcagttcagcctgcagttaacctcagtgaccacagaggatactgctacgtattactgtgccagaacctattacgacgacgccttcaccttccagtactgggggcaagggacgctcgtcacagtgagcagt

SEQ ID NO: 49 - DNA, 363 bp, Synthetic construct
  source 1..363: mol_type = other DNA
  misc_feature 1..363: note = 112_23_HC Nucleotide sequence CDRH
caggtgcagctccaagaaagcggaccgggcctggttaaaccaagtcagagcttatctataacttgtatggtcagtggattcagtattaccaccacaaattattggtggcactggattcggcaaccgcccggcaagcgcctggagtggctgggagaaatgaagtacgacgctcataccaaatacaacccatccgtggagagtcgcctgagcatcactagggatacaggaaaaaatcaattttttctgcagctcacttccgttaccaccgaagataccgcgacttactactgcgccagaacatattatgatgatgccttcacattccaatactggggccaggggacactggtcacggttagctcc

SEQ ID NO: 50 - protein, 9 aa, Synthetic construct
  REGION 1..9: note = Amino acid sequence for CDRL3- VK1-178
QQGNSFPLT

SEQ ID NO: 51 - protein, 13 aa, Synthetic construct
  REGION 1..13: note = Amino acid sequence for CDRL3- VL8-87
GANDGSGSSFVYV

SEQ ID NO: 52 - protein, 9 aa, Synthetic construct
  REGION 1..9: note = Amino acid sequence for CDRL3- VK1-274
LQYGTSPYT

SEQ ID NO: 53 - protein, 9 aa, Synthetic construct
  REGION 1..9: note = Amino acid sequence for CDRL3- VK2-3
FQNTHSRNT

SEQ ID NO: 54 - protein, 9 aa, Synthetic construct
  REGION 1..9: note = Amino acid sequence for CDRL3- VK1-140
QQYDTYPFT

SEQ ID NO: 55 - protein, 8 aa, Synthetic construct
  REGION 1..8: note = Amino acid sequence for CDRL3- VK3-280
FQYYSGFT

SEQ ID NO: 56 - protein, 9 aa, Synthetic construct
  REGION 1..9: note = Amino acid sequence for CDRL3- VK2-16
VQTTHDPYT

SEQ ID NO: 57 - protein, 9 aa, Synthetic construct
  REGION 1..9: note = Amino acid sequence for CDRL3- VK4-81
MQTYSAPWT

SEQ ID NO: 58 - protein, 9 aa, Synthetic construct
  REGION 1..9: note = Amino acid sequence for CDRL3- VK2-16
VQTTHDPNT

SEQ ID NO: 59 - protein, 9 aa, Synthetic construct
  REGION 1..9: note = Amino acid sequence for CDRL3- VK2-16
VQTTHDPNT

SEQ ID NO: 60 - protein, 7 aa, Synthetic construct
  REGION 1..7: note = Amino acid sequence for CDRL3- VK1-115
QHNYGIS

SEQ ID NO: 61 - protein, 9 aa, Synthetic construct
  REGION 1..9: note = Amino acid sequence for CDRL3- VL5-74
GFWHSNRYV

SEQ ID NO: 62 - protein, 9 aa, Synthetic construct
  REGION 1..9: note = Amino acid sequence for CDRL3- VL5-74
GFWHSNRYV

SEQ ID NO: 63 - protein, 9 aa, Synthetic construct
  REGION 1..9: note = Amino acid sequence for CDRL3- VL5-74
GFWHSNRYV

SEQ ID NO: 64 - protein, 9 aa, Synthetic construct
  REGION 1..9: note = Amino acid sequence for CDRL3- VL5-74
GFWHSNRYV

SEQ ID NO: 65 - protein, 9 aa, Synthetic construct
  REGION 1..9: note = Amino acid sequence for CDRL3- VK2-16
VQSTYDPPT

SEQ ID NO: 66 - protein, 9 aa, Synthetic construct
  REGION 1..9: note = Amino acid sequence for CDRL3- VK2-16
VQTTHDPIT

SEQ ID NO: 67 - protein, 9 aa, Synthetic construct
  REGION 1..9: note = Amino acid sequence for CDRL3- VK2-16
VQSTQDPPT

SEQ ID NO: 68 - protein, 9 aa, Synthetic construct
  REGION 1..9: note = Amino acid sequence for CDRL3- VK2-16
VQSTQDPPT

SEQ ID NO: 69 - protein, 9 aa, Synthetic construct
  REGION 1..9: note = Amino acid sequence for CDRL3- VK2-16
VQSTQDPPT

SEQ ID NO: 70 - protein, 9 aa, Synthetic construct
  REGION 1..9: note = Amino acid sequence for CDRL3- VK2-16
VQSTQDPPT

SEQ ID NO: 71 - protein, 9 aa, Synthetic construct
  REGION 1..9: note = Amino acid sequence for CDRL3- VK4-95
QQFISGPPT

SEQ ID NO: 72 - protein, 9 aa, Synthetic construct
  REGION 1..9: note = Amino acid sequence for CDRL3- VK4-95
QQFIRAPPT

SEQ ID NO: 73 - protein, 8 aa, Synthetic construct
  REGION 1..8: note = Amino acid sequence for CDRL3- VK4-95
QQFISAPP

SEQ ID NO: 74 - protein, 9 aa, Synthetic construct
  REGION 1..9: note = Amino acid sequence for CDRL3- VK4-95
QQFISGPPT

SEQ ID NO: 75 - protein, 9 aa, Synthetic construct
  REGION 1..9: note = Amino acid sequence for CDRL3- VK4-95
QQFIRGPPT

SEQ ID NO: 76 - protein, 10 aa, Synthetic construct
  REGION 1..10: note = Amino acid sequence for CDRL3- VK1-140
QQYDSYPPLT

SEQ ID NO: 77 - protein, 10 aa, Synthetic construct
  REGION 1..10: note = Amino acid sequence for CDRL3- VK1-140
QQYDGYSPLT

SEQ ID NO: 78 - protein, 9 aa, Synthetic construct
  REGION 1..9: note = Amino acid sequence for CDRL3- VK2-16
VQTTHDPYT

SEQ ID NO: 79 - protein, 9 aa, Synthetic construct
  REGION 1..9: note = Amino acid sequence for CDRL3- VK1-274
LQTYISPRT

SEQ ID NO: 80 - DNA, 354 bp, Synthetic construct
  source 1..354: mol_type = other DNA
  misc_feature 1..354: note = 109_1_HC Nucleotide sequence CDRH
gaggcccaactggtagagagcggaggagggctggtgcaacctggcgggtctcttagactgtcctgcgtggcttcaggcttcactttcaacaggtatgctatgaactggatccggcaggcccccgggaaaggattggagtggattagcatgatttggtacgacggctccaaaatgtattacgcggatagtgttaaaggcagatttacgatttcccgggataacgataaaaatacactctatttacagatgagctcactgcgcccagaggacaccgccctgtattactgtgccaaggctccctacggatacgagggtgacgtttggggagccggcacattggtgaccgtgtccagt

SEQ ID NO: 81 - DNA, 321 bp, Synthetic construct
  source 1..321: mol_type = other DNA
  misc_feature 1..321: note = 109_1_LC Nucleotide sequence CDRL
tgtggaatacagctcactcaacctcctagcgccagcgctagcgtcggggacactgtcaagatttcctgccaggcatcacaatcagtttcaagctatcttaactggtaccaacagaaacctggccaggcgcctaaacgcttgatttatggcgctaccaacctggaaagtggcgtcccttccaggttttcaggttctggaagcggtactggttttaccctcactatcagctccctgcaacccgaagattttgctacatattactgccagcaaggcaattctttccccttgacctttggcggcggcaccagactggagatcaag

SEQ ID NO: 82 - DNA, 381 bp, Synthetic construct
  source 1..381: mol_type = other DNA
  misc_feature 1..381: note = 109_2_HC Nucleotide sequence CDRH
gaagagcagctcgttgagtcaggcggaggactcgtacagtccggaggctctctcaagctgtcctgcatggcttccggctttacgttttccagctaccctatgagctggattcggcaggcacctggcaaaggcctcgagtggatctcaaccatcggatctcgcgggtcaaaaacttactacgccggctccgtgaagggccgtttcacaatcagcagggacaatggcaagaatacgttgtacctccagatgagtagtttgcgccctgaagacacggcagtttattactgcgcccggtactcctattaccgctatagtgatggaacatattactccagcggcttcgacgtttggggagctgggaccttggtgaccgtctcctcc

SEQ ID NO: 83 - DNA, 348 bp, Synthetic construct
  source 1..348: mol_type = other DNA
  misc_feature 1..348: note = 109_2_LC Nucleotide sequence CDRL
cagcctgtgctgacccaactgccttcagcaagcgcctcagtggggcagacattaaagctgacttgtaccctgagcagcgggtatagcaactataatgtgaattggtaccaacaatcacaggggaagtctccccgctttgtgatgcgcgtcggaactagcggtatagtggggagcaagggagacggcataccagataggttttcaggtagtggtagcggctgggatcgatatttgactatccagaacatccaggaggaggatgagaacgtttactattgtggcgctaacgatggctctggctcttcctttgtctacgtcttcgggggaggaaccaaactcacggtcctt

SEQ ID NO: 84 - DNA, 366 bp, Synthetic construct
  source 1..366: mol_type = other DNA
  misc_feature 1..366: note = 109_3_HC Nucleotide sequence CDRH
gaggaacagctggtagaaagtggagggggactggttcaaccgggcggatcactgaagctttcttgcgtggcgagtggattcacatttagcgactattggatgagctggatccggcaggctcctggtcgcggtttggagtggctgacctacataaattatgacggcgatgatacgtactacacagacagcgtgaaggggcggtttaccatctcccgagacaactcaaggaatgtcctgttcttgcagatgtcacgcctcaagcccgaagataccgctgtgtattactgcgctcgggtgcccttttatggataccattatggggccccagacgtgtggggtgcgggcaccctcgtgacagtgtcctct

SEQ ID NO: 85 - DNA, 321 bp, Synthetic construct
  source 1..321: mol_type = other DNA
  misc_feature 1..321: note = 109_3_LC Nucleotide sequence CDRL
tgtgacattcagctcacacagccggcctccgcgagcgccagcgtgggcgacactgtgaagattagctgtcaggccagccagaatatctatagctatctctcttggtaccagcaaaagcccggacaggccccgaagctccttatctactatgcgaaccgcctcgcaagcggcgtcccgtcacgcttcagcggagcagggagtggaactgactttacactcacaatctcttccctgcagcctgaagattttgccacatactattgtttgcaatacggcacatcaccttacaccttcggcgccggcaccaaactggaaataaag

SEQ ID NO: 86 - DNA, 357 bp, Synthetic construct
  source 1..357: mol_type = other DNA
  misc_feature 1..357: note = 109_4_HC Nucleotide sequence CDRH
gaggtgcagctggtggagtccggcggggggctggtgaagccaggcgagtcacttcgcctttcttgcgtggctagcggttttacctttagctcatactggatgaactggatccgtcaggcccctggtaaagggctggagtggatcagcgccctgaatagcgatggcagcaccactctttacgcggacagtgtcaagggccggtttaccattagccgagataacggtaagaacaccctctacctgcaaatgagctcactgagacctgaggacacagcaatctactattgcacgctgacttattactggagtgcagacttcgacgtctggggcgcaggtaccctcgtgacagtttcaagc

SEQ ID NO: 87 - DNA, 336 bp, Synthetic construct
  source 1..336: mol_type = other DNA
  misc_feature 1..336: note = 109_4_LC Nucleotide sequence CDRL
gacgtggtaatgacacaaacccccctgtcactgtccgtcagtccaggtgaaccagcgtccatttcctgtagagctagccagtcactgcttcactccaatggtaagacatatcttcattgggtggttcataaaccagggcaagctccacgcggcatgatctaccaggtatcaaataagtatagtggaaccccagagcgcttcagtggaagtgggtccgggactgatttcactctcaagataagtcgcgtggaagcagaagacgccggagtgtactattgtttccagaacactcattccagaaatacttttgggggcggcacaaaattagagattaag

SEQ ID NO: 88 - DNA, 363 bp, Synthetic construct
  source 1..363: mol_type = other DNA
  misc_feature 1..363: note = 109_5_HC Nucleotide sequence CDRH
caagtgcagctgcaggaatcaggacccggccttgtcaagccctctcagagcctcagcatcacctgcatggtgtctggatttagcatcacaaccacaaactattggtggcactggatcaggcagccacctggcaaatccctggaatggctgggggaaatcagctacgacggcaataccaaaagcaacccatcagtcaagtcaagactgtctatcactagggacaccggcaaaaatcagttctccctccagctgaccagcgtgacaacggaagacacggctacatactattgtgcgcgcggatattacgacgacagttttaatttccagtactggggacagggcaccctggttactgtgtcttcc

SEQ ID NO: 89 - DNA, 321 bp, Synthetic construct
  source 1..321: mol_type = other DNA
  misc_feature 1..321: note = 109_5_LC Nucleotide sequence CDRL
gatatccagatgactcaaagccccagtttcctttcagcatctcttggggaccgagtaaccatcacttgtaaggcatctcaaaacattcatcggaatctggcctggtaccaccagaaacctggaaaagccccccggctgttgatttatagtacaagcaacctggactcaggtgtgccttctcgcttctccgggagcggcagttggactgactacgcatttactatttcagctgtcgagcccgacgatgtaggaatttatcattgtcaacagtatgatacttacccttttacattcggagggggcactaagctcgaaatcaag

SEQ ID NO: 90 - DNA, 369 bp, Synthetic construct
  source 1..369: mol_type = other DNA
  misc_feature 1..369: note = 112_1_HC Nucleotide sequence CDRH
gaggtccagctggtggaaagtggtggcggcttggtgcaaccagggggctctctgagactgagctgtgtcgcttccggattttccttcgacaattatggtatgacctggattaggcaagcccctggcaaaggcttggaatggattagcgccatcagcttctctgggggtaacactaattatgcagacagtgtgaaggggaggttcacaatttcccgcgataacggaaagaacgctctctacctccagctctcctcactccgacctgaggacaccgccacttattattgcgctcgagactattcaagcggcttcttcccacgctccgggttccaacactggggacagggaacgttggtgaccgtgtcctct

SEQ ID NO: 91 - DNA, 318 bp, Synthetic construct
  source 1..318: mol_type = other DNA
  misc_feature 1..318: note = 112_1_LC Nucleotide sequence CDRL
caaacactgttgacacaaactccagatagtttagcggtgtctcccggcgagacggttaccctgaattgtcgggcatcccagggggtctcagactacctcgagtggtatcaacagaagcccggacgtgcccctcgcctgctcatatactcagccagtgacagagctaccggggtgcctgcccgctttagcggaagcggatctgggacagattttactttaacaatttccagtctgcaaccacaggacattgcggtctattattgttttcagtactattcaggatttacctttggtggtgggactaaattagaaattaaa

SEQ ID NO: 92 - DNA, 357 bp, Synthetic construct
  source 1..357: mol_type = other DNA
  misc_feature 1..357: note = 112_2_HC Nucleotide sequence CDRH
caggtccagcttcaggagagtggatccggcttagtgggcccatcccagactctttccctcgcatgttctgtgagcgggttcagcattaccacaagcggatactcttgggcttggattcggcaacctcccggcaagtcactggagtggatgggccacatttggtacgatgccgacacttactacaacccctccatcaagaacaggattagcatttcccgtgacaccgggaaaaatcagttttccctccagctcagctccatcaccacggaggatacagcaatctattactgtgctcgatgtggggcctactatgattttgacgtctggggggccggcactttggtgactgtaagctca

SEQ ID NO: 93 - DNA, 336 bp, Synthetic construct
  source 1..336: mol_type = other DNA
  misc_feature 1..336: note = 112_2_LC Nucleotide sequence CDRL
gacgtcgtaatgacacagactccattgctgctgtccgtctctcccggagagcctgccagcatgtcctgcaagagcagccaaagcctgtggaactcagatggttacacttacttacactggctggtgcataaaccaggccaggccccacggggcgtgatctatgaagtgtctgataaatattcatggaccagcgaacggtttacaggcgggggatccggaacagactttactctgaagatatctcgcgtagaggccgaggatgccggggtgtactactgcgtgcagacaacccatgacccatatacttttggagcggggactaaactcgagattcgg

SEQ ID NO: 94 - DNA, 363 bp, Synthetic construct
  source 1..363: mol_type = other DNA
  misc_feature 1..363: note = 112_3_HC Nucleotide sequence CDRH
gaggtccagctggttgagtccggaggtgggctcgctcagccgggtggttccttgcgactcagctgcttggcgagcggttttactttcaacgagtcttggatgtcttgggtgcggcaggtgcccggcaaggcccttgagtggctggcaaacattaacggagatagtaccaaaatgaactacgctgatagcgtcaaaggtagattcaccatgtccagggactctggcaaaaacaccctgtaccttcaaatgaacggcttgcgaagcgccgacaccgccttgtactactgtgccgccgaatattactcacgcgacaactattatgaaataatttggggcgccggaactctggtgaccgtatcttcc

SEQ ID NO: 95 - DNA, 339 bp, Synthetic construct
  source 1..339: mol_type = other DNA
  misc_feature 1..339: note = 112_3_LC Nucleotide sequence CDRL
gacatcgtgatgactcagtcccctgcttcactggcagtgacacctggtgagtccgccaccatacactgtaagagctctcaatccctgctgtattcatatgacaacaaaaactacctcatatggtatcagcaaaagccaggccagtctccaaaattgttgatttctttagcctctacccgtaacacaggagtctcagatcactttagcgggtctggttctgggaccgacttcacactcacaatctcaagggtgcaggctgaggatgttgcggattattactgcatgcaaacgtactccgcaccatggacattcggcgctggaacaaagttagagattaag

SEQ ID NO: 96 - DNA, 339 bp, Synthetic construct
  source 1..339: mol_type = other DNA
  misc_feature 1..339: note = 112_4_HC Nucleotide sequence CDRH
gaggtacaactggttgagtcaggggggggattggtgaaaccaggcgaaagccttcgcttgtcttgtgtggctagtggattcacgttctcctcatactggatgaactggattcgccaggctccagggaaggggctcgagtggatctctaccatcaattccgacggcactaatacttattattccgacagtgtgaaaggcagattcaccattagtcgcgataatgggaagaatactctgtacctccagatgagcagcttgcgtcctgaagatactgccatgttctattgcgctactggcctggccgtgtggggcgccgggactctggtgactgtttcatca

SEQ ID NO: 97 - DNA, 336 bp, Synthetic construct
  source 1..336: mol_type = other DNA
  misc_feature 1..336: note = 112_4_LC Nucleotide sequence CDRL
gatgtggtcatgacacagaccccactccttttgagtgtgtctcccggcgagcctgcgtctatttcatgtcggagttcccagtccctcttatatagcgacggctccaccctgctgcactggttagtacataagccaggccaagccccaaggggagtgatctacgaagttagcgataagtactcctggacccctgaaaggttcacgggctctggctcaggaaccgattttacacttaagatctctcgggtcgaggctgaggacgctggagtctactactgcgttcagactacgcacgatcccaacaccttcggcggaggcaccaagctcgaaataaag

SEQ ID NO: 98 - DNA, 352 bp, Synthetic construct
  source 1..352: mol_type = other DNA
  misc_feature 1..352: note = 112_7_HC Nucleotide sequence CDRH
caggtccaattgcaggaatccggcccaggacttgtgaagccttcacagacactgagcctgacatgcacagtgacaggcttttccatcacaacgaacacgtattattggtcctggatccgccaacctcctggtaaatcacccgaatggctcggtattatagacagcggaggtgacacgacctacagcccctcattgaagagccgaattagtatctctagggacactggcaaaagtcagttttctcttcagctgcgttccgtgactcctgaggacacagccacttattactgcgctactctgacaaccacgggagaatattggggtcagggtaccctggtaactgtgtcatcag

SEQ ID NO: 99 - DNA, 333 bp, Synthetic construct
  source 1..333: mol_type = other DNA
  misc_feature 1..333: note = 112_7_LC Nucleotide sequence CDRL
caggagttagtgactcaagaacctagtctctccgcaccccccggaacggcagtccgcttaacttgcacactgcggtccgatcttagttcctaccgcattttctggtaccagcagaagcccgggtctcccccccggttcctccttcattaccataccgattctgacaagcagctcggctctgggttcccttctcggtttagtggcagtaccagcgccaatgctggtgtgttacggatttccggtctccagcccgaggatgaggcggacttctattgcggcttttggcacagtaaccgatatgtgtttggtggcggcactaaacttacagtgctc

SEQ ID NO: 100 - DNA, 351 bp, Synthetic construct
  source 1..351: mol_type = other DNA
  misc_feature 1..351: note = 112_8_HC Nucleotide sequence CDRH
caggtgcaactccaggaatccggacccggattagtgaagcctagccagaccctcagcctcacatgtaccgggactggagtctctatcaccgccagcacttattactggagctggattcgccaacccccaggtaagagtcccgagtggatgggcatcatcgactcagacggcgacacagcatattctccatctctcaaaagccgaatatctatctctagagatactggtaagaaccagttcagccttcagctcaggagcgtcactccagaggatacagctacatacttctgtacaacactgacaaccactggcgagtattggggccagggaactctggtgaccgtcagctca

SEQ ID NO: 101 - DNA, 333 bp, Synthetic construct
  source 1..333: mol_type = other DNA
  misc_feature 1..333: note = 112_8_LC Nucleotide sequence CDRL
caggaactggttacccaggagccttccctgtctgcccccccaggcactgccgctcgcttgacctgcacattaaggagtgacctttcttcttacaggatcttctggtatcagcagaaaccaggctctcctccgagatttctgttgcactatcatactgactctgataagcagttgggcagcggcttcccaagccggttctctggatccacttccgcgaatgccggggttttaggaattagtgggctgcagccagaggatgaggccgattactattgcggattttggcatagcaacaggtatgtgtttggtggtggaacaaaactgaccgtactt

SEQ ID NO: 102 - DNA, 351 bp, Synthetic construct
  source 1..351: mol_type = other DNA
  misc_feature 1..351: note = 112_9_HC Nucleotide sequence CDRH
caggtgcagctgcaagaatccgggcccggacttgtgaagccttctcaaaccctctcactgacctgtacagggacaggagtgtctatcaccgctaatacttactattggagctggattcgccagccccctgggaaatctcctgagtggatgggaataattgacagcgacggcgacacggcatacagcccttctctgaaatcccggatatccatctccagagacactggtaagaatcagttcagcctgcagttacgctccgtgacccctgaggatactgcaacctatttttgcaccacgctgacaacaactggcgagtattggggccagggaactctcgttacagtgtcctct

SEQ ID NO: 103 - DNA, 333 bp, Synthetic construct
  source 1..333: mol_type = other DNA
  misc_feature 1..333: note = 112_9_LC Nucleotide sequence CDRL
caagagctcgttacgcaggagcccagcctgtccgcccccccaggcaccgccgcaaggctgacatgtaccctgagaagtgatctcagcagctataggatcttctggtaccaacagaaacctgcctcccccccacgctttttgctccactatcataccgatagtgataagcagctcggctccggcttcccttctcgattttcaggaagcacaagcgctaacgccggggttctcggtatctccggtctgcagcctgaggatgaagctgactattattgtggtttctggcatagtaacaggtacgtcttcgggggcggtacaaaactcacagtgctg

SEQ ID NO: 104 - DNA, 351 bp, Synthetic construct
  source 1..351: mol_type = other DNA
  misc_feature 1..351: note = 112_10_HC Nucleotide sequence CDRH
caggtccagctgcaggaaagtggtcccggactggtgaaaccttcacaaacactcagcctgacctgtacaggaactggggttagtattacagcaagtacttattactggtcatggatcaggcagccacccggtaagtctcctgagtggatgggtattattgacagtgatggggacacggcttacagcccaagcttgaagtcaagaatatccatcagcagggatacaggaaaaaatcaattctctcttcaacttcgtagcgtggcacccgaggataccgccacgtatttctgtacaactcttactacaactggtgagtactggggccaggggactttagttaccgtctcctct

SEQ ID NO: 105 - DNA, 333 bp, Synthetic construct
  source 1..333: mol_type = other DNA
  misc_feature 1..333: note = 112_10_LC Nucleotide sequence CDRL
caggaactggtaacccaggagtcttctctgagcgccccacccgggaccgctgcacgactgacttgcactctccgcagcgacctgagttcctatcgcatcttctggtatcagcagaaaccaggctctcctcctagatttctgctgcactaccacacagacagtgataagcagttaggaagcggcttccctagccggttctctgggtcaacctcagcaaatgccggggttctgggcatctcaggccttcagcctgaggacgaggctgcatattattgtgggttttggcattctaatcgctacgtgttcggcgggggcaccaagctgacagtgctc

SEQ ID NO: 106 - DNA, 354 bp, Synthetic construct
  source 1..354: mol_type = other DNA
  misc_feature 1..354: note = 112_11_HC Nucleotide sequence CDRH
gaagagcatctcgtcgagagcggcggcggcctggttcagcctggggggcccctccagctgagctgcgtcgcaagtggcttcactttttctaactatgcaatgagttggatcagacaagcgcctgggaaaggcctggaatggatttcagcgataaatagcgtctctaagatctattacgcggattccgtcaagggcaggttcacaatcagcagggacaacgacaaaaacacgctctacctccagatgagctccttaaggccagaggacaccgcagtgtactactgcgctagggaagaaggcgttcaggtttacttcgatgtctggggagcgggcacgctggtcactgtgtcttct

SEQ ID NO: 107 - DNA, 336 bp, Synthetic construct
  source 1..336: mol_type = other DNA
  misc_feature 1..336: note = 112_11_LC Nucleotide sequence CDRL
gacgtcgtgatgacccagactccgctgctgctgagcgtatctcctggtgagccagcctcaatcagctgtcgaagctcccagtccttgctctattctgacggatatacttatctccattggcttgttcacaagcctgggcaggctccacgcggagttatatacgaggtgagcgacaaatattcctggactcccgagcgattcaccgggagtggctctgggactgacttcactctgaagatcaaccgggtggaggctgaagacgctggtctgtattactgcgttcagagtacctatgaccctcctacttttggggccggtacaaaactggagattaag

SEQ ID NO: 108 - DNA, 339 bp, Synthetic construct
  source 1..339: mol_type = other DNA
  misc_feature 1..339: note = 112_12_HC Nucleotide sequence CDRH
gaggttcagctcgtcgagtcaggcggaggattagtgaaacctggggagtccctcaggctcagttgtgtggctagcggcttcgccttctccacctactggatgaattgggttagacaggcacccgggaaagggttagaatggatttcaggcatctccggagacggctcaagcacctattacgccgactcagtgcaggggcagttcacaatctcccgcgataacgacaagaacactctgtccctccagatgtcttctttaagacctgaagacacagcaatgtattactgcacttccggcatcagtcagtggggtgccgggacacttgtgaccgtgtcatct

SEQ ID NO: 109 - DNA, 336 bp, Synthetic construct
  source 1..336: mol_type = other DNA
  misc_feature 1..336: note = 112_12_LC Nucleotide sequence CDRL
gacgtcgtcatgacgcagacgcccctgcttttgtccgtgtcacccggtgagcccgcttcaatttcctgcaggagctcccaatcccttctcgattctgacggatataactacctgcactgggtcgttcacaagcccgggcaggccccccgcggagtgatctacgaggtctctgacaagtatagctggaccccagagcggtttactggatccggcagccgaacagacttcacactcaaaattagtagggtggaagccgaagacgccggagtctactactgtgtacagacaactcacgaccccataacttttggcggggggactaagctcgaaatcaaa

SEQ ID NO: 110 - DNA, 357 bp, Synthetic construct
  source 1..357: mol_type = other DNA
  misc_feature 1..357: note = 112_13_HC Nucleotide sequence CDRH
gaggagcagctggtcgaatccggtgggggactggtgcagccaggcggctccctgaaactgtcttgcatggcttccggctttacatttagctcctacgccatgacctggataaggcaggcaccaggcaagggtcttgagtggatcagtgccattagatcctcagggtcaaacacttattatgctggaagcgtaaaaggccgttttaccataagcagggataatgataagaataccttgtacctgcagatgtctagcctcagaccagaggacacagccgtctactattgcgttcgcgaggaagggggacaggtctattttgacgtttggggtgcggggaccctcgtgaccgtgtcatct

SEQ ID NO: 111 - DNA, 336 bp, Synthetic construct
  source 1..336: mol_type = other DNA
  misc_feature 1..336: note = 112_13_LC Nucleotide sequence CDRL
gatgtggttatgacgcagacgccgctgctcctgagcgtgtcacccggcgaacccgcctctattagttgtaggtcctcccaatccctgttatatagcgacggctatacatacttgcactggctggtgcacaaacccggtcaggccccccgtggagtgatctacgaggtgtccgacaaatatagttggactccagagcgattcagtggctccggctctggtacggattttacgttaaagatttctcgggtggaggccgaggacatcggcgtgtactactgtgtgcagtctactcaagacccccccactttcggagctgggactaaactggaaatcaaa

SEQ ID NO: 112 - DNA, 357 bp, Synthetic construct
  source 1..357: mol_type = other DNA
  misc_feature 1..357: note = 112_14_HC Nucleotide sequence CDRH
gaggaacaattagtcgagtcaggagggggacttgttcagcccggcgggtcactgaaattaagctgcatggcctcaggtttcagctttagctcctacgccatgacatggatacgtcaggcaccaggaaagggcctggaatggatcagcgccatcaggtcctccggcagtaacacttactacgcggactctgtcaaagggcgttttacaatctctcgggataatgacaagaatatgctctatctgcagatgtcctccctgaggccggaggacaccgcagtgtactattgtgtacgcgaagaaggaggccaggtgtacttcgacgtgtggggtgccggtactctggtcaccgtctcctcc

SEQ ID NO: 113 - DNA, 336 bp, Synthetic construct
  source 1..336: mol_type = other DNA
  misc_feature 1..336: note = 112_14_LC Nucleotide sequence CDRL
gagatcgtcatgacccagacgccattgctgctctcagtttctccaggggagcccgcaagcattagctgccgttcatctcagtctctgctgtacagtgacggttacacttacctccactggctcgttcataagcctgggcaggcaccccgcggagtgatttacgaggtgtcagacaagtattcctggacaccagaacgattcacggggtcaggatccggaacagacttcacattgaagatttcccgcgtggaagcagaagacattggggtgtattattgtgtccagtcaacccaggatccccctacgttcggggcaggcaccaagctcgaaatcaag

SEQ ID NO: 114 - DNA, 358 bp, Synthetic construct
  source 1..358: mol_type = other DNA
  misc_feature 1..358: note = 112_15_HC Nucleotide sequence CDRH
gaggagcagctcgtcgagagtggcgggggactggtgcagccaggaggatctcttaaactctcctgtatggccagtggatttagtttttcatcctacgccatgacctggattaggcagggtcccgggaagggcctggaatggatcagcgctattcgttcttctggcagcaagacatattatactggaagcgtgaaaggtcgctttactatttccagagataatgacaagaacatgctctatttacagatgtccagcctgaggccagaggatacagcagtgtactattgtgtacgggaggagggggggcaagtgtactttgatctctggggggcaggtaccttggtcaccgtgtcctctg

SEQ ID NO: 115 - DNA, 336 bp, Synthetic construct
  source 1..336: mol_type = other DNA
  misc_feature 1..336: note = 112_15_LC Nucleotide sequence CDRL
gagattgttatgactcagactcccctgctgctgagcgtgtcaccgggcgaacctgcatcaattagttgccgcagttctcagagcctcctctattcagacggctacacatacttgcattggttggttcacaagcccggacaagcaccaaggggcgttatctatgaagtctccgacaaatactcctggactcctgaacgcttcaccggctctggttccggtacagatttcacccttaaaatctccagagtagaggcagaggacataggagtttactactgtgtgcagtcaactcaggaccccccgacattcggtgcaggtacaaaactcgaaatcaag

SEQ ID NO: 116 - DNA, 357 bp, Synthetic construct
  source 1..357: mol_type = other DNA
  misc_feature 1..357: note = 112_16_HC Nucleotide sequence CDRH
gaggagcagctggtggaatccggtggtggattggtccagcccggtggcagtctgaaacttagctgcatggcctccggcttctccttctcctcttatgcaatgacttggatccggcaggcacctgggaaaggacttgaatggataagcgctattagaagcagcggctctaatacgtactacgccgacagcgttaaagggagattcaccattagtcgagataacgacaaaaacatgttgtacttacagatgagctctttaaggcctgaagacacagccgtttactattgtgtacgtgaggagggcggacaagtctattttgacgtctggggagccggtacgttggtgactgttagctcc

SEQ ID NO: 117 - DNA, 336 bp, Synthetic construct
  source 1..336: mol_type = other DNA
  misc_feature 1..336: note = 112_16_LC Nucleotide sequence CDRL
gaaattgtaatgactcagacccccttgctgcttagcgtcagccctggtgagccggcatccattagctgtcggagttcacaaagtctgctgtacagcgatggttacacctacctccactggcttgttcacaagccgggacaggccccacggggggtgatttatgaagtgtcagataagtattcttggactcctgaacgcttcacaggatccggaagcggcaccgactttactcttaagatcagcagagtagaggcagaggacatcggcgtctactattgtgtgcagtcaacgcaagatcccccaactttcggtgccggcaccaagctcgaaatcaaa

SEQ ID NO: 118 - DNA, 351 bp, Synthetic construct
  source 1..351: mol_type = other DNA
  misc_feature 1..351: note = 112_17_HC Nucleotide sequence CDRH
cagatgcagctgcaagaaagcgggccagggctcgtgaagccgagccagactctgtttttgacctgtagcgttagcgggttttccataaccacgtccgggagtggctggagttggattcggcaaccccgcgggaagactctggaggttttagggggagttgcatacaacggagggactggctataatccatccatagaatcccggatttccatatctagggatactggcaaaaaccaatttagcctgcaggtgaactcagtgaccgaagaagacaccgccacatactattgcgctagagggatgggatatttcgacgtttggggggcgggtactctggttactgtgagctca

SEQ ID NO: 119 - DNA, 339 bp, Synthetic construct
  source 1..339: mol_type = other DNA
  misc_feature 1..339: note = 112_17_LC Nucleotide sequence CDRL
gagacagtgatgacacagtctcccgccactatggccgtttcaccaggtgagagagtaaccatacattgtaaatccagccagtctctgttttacagcggtaacaataagcatctccttagctggtatcagcagaagcctggacagagccctaagctgctcatttacttcacgtccacgagagccagtggagtgcctgaccggttttccggatccgggtctggaaccgacttcactctcacaattaattccctgcaggccgaggacgtagccgattatttttgccagcagttcatttctgggccacctacattcggggctggaacaaaattggaaatcaag

SEQ ID NO: 120 - DNA, 351 bp, Synthetic construct
  source 1..351: mol_type = other DNA
  misc_feature 1..351: note = 112_18_HC Nucleotide sequence CDRH
cagatgcatctgcaggagagcggccccggcctcgtcaaaccatcacagacgttgttcctgacatgttcagttagcgggtttagtatcacaacaagtgcctatggctggagctggatacggcaacccagaggcaaaaccctggaagtaatgggtggcattgcctataatggaggaacaggctataacccaagtatcaaatcccggatttccatatccagggacacaggtaagaaccaattttcacttcagctcaacagcgtgaccgaggaagacacggcaacctactattgcgctaggggaatggggtactttgatgtgtggggcgccggaacactggttaccgtaagctcc

SEQ ID NO: 121 - DNA, 339 bp, Synthetic construct
  source 1..339: mol_type = other DNA
  misc_feature 1..339: note = 112_18_LC Nucleotide sequence CDRL
gatatcgttatgactcaaagtccagcaaccatggccgtgtctcccggagagagagtgactatccactgcaagtcctcccagagtctcttctattcaggcaattccaaacacctgttatcatggtatcagcagagacccggtcagtccccgaagctgctgatcagttgggcatccacccgcgcgagtggagtccctgatagattcagcgggtctggctctggtacagacttcacacttacgatctcttcactccaggctgaagacgtagcagtgtacttctgtcagcagtttatccgagcccctcccacatttggagccggcactaaactcgaaattaag

SEQ ID NO: 122 - DNA, 351 bp, Synthetic construct
  source 1..351: mol_type = other DNA
  misc_feature 1..351: note = 112_19_HC Nucleotide sequence CDRH
cagatgcagttacaggaatcaggtcctggccttgtgaagccatcccagaccctttttctcacctgttccgtgtcaggcttcagtattactacgagtggatacggatggagctggattaggcagccacggggtaaaacactggaggtcatgggcggaatcgcatataacggggcgacaggctacaacccaagtattaagagcaggatctctatctccagagataccggcaaaaatcaattttcactccagttaaattctgttactgaagaggatacggccacttactactgcgtacgaggaatgggttactttgatgtctggggcgcaggaacattggtgacagtctctagt

SEQ ID NO: 123 - DNA, 339 bp, Synthetic construct
  source 1..339: mol_type = other DNA
  misc_feature 1..339: note = 112_19_LC Nucleotide sequence CDRL
gacatcgttatgacccagtcccccgcaacaatggctgtttccccaggtgagagagtgaccatccattgtaagagcagccaaagcctgttttattccggaaataataaacacctcctgagttggtaccagcaaaagcccggccagtctcctaaactgttgatctattggccttcaactagggcctcaggcgttccagatcgcttcagtggatccggatccggtacagatttcactctcaccattagttccctccaagccgaggatgttgcagattacttttgtcagcagtttatcagcgctccccctacattcggcgccgggacaaagcttgaaataaaa

SEQ ID NO: 124 - DNA, 351 bp, Synthetic construct
  source 1..351: mol_type = other DNA
  misc_feature 1..351: note = 112_20_HC Nucleotide sequence CDRH
cagatgcaactgcaggagagtggccccggacttgtaaagccttctcaaacgctgtttctcacttgcagtgtgtccggcttcagcatcacaacatctggaagcgggtggtcctggatcagacaaccacgcgggaaaactttagaagtgatgggactgattgcctacaatggagataccggttataaccctagtatcaaatctcgcctcagtataagtcgggatacaggaaaaaaccagtttagccttcaattgaacagtgtgactgaagaagatactgctacctactactgtgcgagggggggagggtactttgacgtctggggcgctggcaccttagtgacagtctcctcc

SEQ ID NO: 125 - DNA, 339 bp, Synthetic construct
  source 1..339: mol_type = other DNA
  misc_feature 1..339: note = 112_20_LC Nucleotide sequence CDRL
gatatcgtgatgacacagagcccggctaccatggccgtttctccaggcgagcgggtgaccatccattgtaagagtagtcagtctttgttttactccggcaacaataaatatttgttgagttggtaccagcagaacccaggtcagtctccgcgcctgctgatctactgggcttctacacgcgctagtggagtacctgacagattcagcgcatccggatcaggcaccgatttcaccttgactatctccagcctccaggccgaagacgtcgcagactatttctgccagcagtttatctccggccccccaacgttcggggccggcacaaagctcgagatcaag

SEQ ID NO: 126 - DNA, 351 bp, Synthetic construct
  source 1..351: mol_type = other DNA
  misc_feature 1..351: note = 112_21_HC Nucleotide sequence CDRH
cagatgcagttacaggagtcaggaccgggactggttaagccttcccagaccctgtttctcacatgcagcgtgtctggtttttcaatcactaccagcggctctgggtggagctggataaggcagccaaggggaaagaccctggaagttatgggagggtttgcctacaatggagacacggcttataacccgtccatcacatccagaatttccatctctagggacacagggaagaatcaattttctctgcagctcaattccgtgactgaagaggataccgccacttactattgtgcgagaggcatgggctactttgacgtctggggggcaggcacactggttacagtctcatct

SEQ ID NO: 127 - DNA, 339 bp, Synthetic construct
  source 1..339: mol_type = other DNA
  misc_feature 1..339: note = 112_21_LC Nucleotide sequence CDRL
gacatcgtcatgacccagtccccggccacaatggccgtctctccaggggagagagctacaatacactgtaagtcatcccaaaccctgttttactctgggaataaaaaacacctccttacctggtatcaacagaagccaggccagtcacccaaaatgattattaattgggcatccacgcgagcaagcggcgtgcctgataggtttagcggctcaggcagtggaacagattttactctcacaatcaactctctgcaggccgaagacgtggccgactatttttgtcagcaattcatccgcggaccgccaacgttcggcgcaggaacaaaattagaaatcaag

SEQ ID NO: 128 - DNA, 324 bp, Synthetic construct
  source 1..324: mol_type = other DNA
  misc_feature 1..324: note = 112_22_LC Nucleotide sequence CDRL
gacatacagatgacacaatccccctcattcctttctgcgtccctcggggaccgcgtcacaattacctgcaaggcatctcagaacatttatcgcaacttggcctggtaccaccagaaacctggcaaggcccccaggctgcttatctacagtgcctctaatctggactctggggtcccgtctagatttagcggattcggctcaggcaccgactacacatttatcattggcgccgtagaatcagatgacgtgggcatctaccactgtcagcagtacgatagttatcctcctctgacattcggtggcggtacgaggctggaaattaaa

SEQ ID NO: 129 - DNA, 324 bp, Synthetic construct
  source 1..324: mol_type = other DNA
  misc_feature 1..324: note = 112_23_LC Nucleotide sequence CDRL
gacattcagatgacacaatcaccctcctttttatcagccagcctcggagatcgggtgacaatttcttgtaaggcttctcagaatattcagcgtaacctggcctggtaccaccagaggccgggcaaggcccctagactgctgatatatgccgccagtagtctggacagtggggtgcctagccgtttttcagggtcaggctccggtacagattatacttttaccatttctaccgtggagccagatgacgtagggatttatcactgtcagcagtacgacgggtacagtcccctgacgttcggaggcggaacccggttggagatcaag

SEQ ID NO: 130 - DNA, 372 bp, Synthetic construct
  source 1..372: mol_type = other DNA
  misc_feature 1..372: note = 112_25_HC Nucleotide sequence CDRH
caggtccaattgcaggaatctggacctggactggtaaaaccaagccaagccctgtcactgacatgtgcggtatctggattttccattactaccacctactatggatggcactggtttaggcagccgaaagggaagtctccagaatggatgggtgtgatcgcttataacggggacacggggtacaatccctctctgcagtcacgaatcagtatttccagagaagctggcaagaatcagttctttctgaagctgcacagcgtgaccgaagaggatacggcgatgtattactgcgtgctgcggggcactagaatggtcacgatcgacccgcattttgatgtgtggggcgcaggaacgcttgttaccgtgtcttcc

SEQ ID NO: 131 - DNA, 321 bp, Synthetic construct
  source 1..321: mol_type = other DNA
  misc_feature 1..321: note = 112_25_LC Nucleotide sequence CDRL
tgcgacgtgttactgacccagcccgctagcgctagtgcatctgttggggataccgtaaaaatttcctgccgagcctcccaaagcgtaggggactttctgtcttggtaccagcagaaggcgggaaaggccccccaactgttgatttatgccgccacccacttggcctatggcgtgtccagcagattctctggctctggcagtggaacggacttcaccctcactataagtagcttgcagcctgaggactttggaacatactattgtctgcagacttatatctctccacgaacctttggtgccgggacaaacctcgagataaaa

SEQ ID NO: 132 - protein, 111 aa, Synthetic construct
  REGION 1..111: note = Amino acid sequence- VH1-58-54+JH2
QVQLQESGPGLVKPSQLSITCTVSGFSITTTNYWWHWICQPPGKSLEWLGEISYDGNTKYNPSVKSRLSIARDTGKNQFSLQLTSVTTEDTATYYCNFQYWGQGTLVTVSS

SEQ ID NO: 133 - protein, 92 aa, Synthetic construct
  REGION 1..92: note = Amino acid sequence- VK1-140+JK2
DIQMTQSPSSLSASLGDRVTIITCKASLAWYHQKPGKAPRLLIYNLDSGVPSRFSGSGSGTDFTLTISAVEPEDVGVYYCNTFGGGTKLEIK

SEQ ID NO: 134 - protein, 111 aa, Synthetic construct
  REGION 1..111: note = Amino acid sequence- VH1-151-54+JH2
QVQLQESGPGLVKPSQTLSLCTCVTGGFSITTSSYYWSWIRQPPGKSPEWMGVIDYDGDTAYSPSLKSRISISRDTGKQFSLQLRSVTEDTATYYCNFQYWGQGTLVTVSS

SEQ ID NO: 135 - protein, 110 aa, Synthetic construct
  REGION 1..110: note = Amino acid sequence- VL5-74+JL3/6/10
QELVTQEPSLSPPGTAARLTCTLRSDLSVGGSYRIFWYQQKPGSPPRFLLHYHTDSDKQLGSGPSRFSRSGSKDTSANAGVLRISGLQPEDEADYYCYVFGGGGTKLTVL

(19) United States
(12) Patent Application Publication
(10) Pub. No.: US 2025/0102520 A1
(43) Pub. Date: Mar. 27, 2025

(54) SIMULTANEOUS DETECTION OF ANTIGENS AND ANTIGEN SPECIFIC ANTIBODIES IN A GUINEA PIG MODEL SYSTEM

(71) Applicant: VANDERBILT UNIVERSITY, Nashville, TN (US)

(72) Inventors: Ivelin Georgiev, Nashville, TN (US); Matthew Vukovich, Nashville, TN (US)

(21) Appl. No.: 18/891,353

(22) Filed: Sep. 20, 2024

Related U.S. Application Data
(60) Provisional application No. 63/584,618, filed on Sep. 22, 2023.

(51) Int. Cl. G01N 33/68; A61K 39/145; A61K 39/21; C12N 7/00; C12N 15/10; C12Q 1/686; C12Q 1/6869; G16B 30/10

(52) U.S. Cl. CPC G01N 33/6854; A61K 39/145; A61K 39/21; C12N 7/00; C12N 15/1096; C12Q 1/686; C12Q 1/6869; G16B 30/10; C12N 2740/16033; C12N 2760/16134; C12N 2760/18534; G01N 2333/11; G01N 2333/16

(57) ABSTRACT

Disclosed herein are methods of determining broadly neutralizing antibodies, antigen specific cell sorting and developing vaccine compositions in a guinea pig model system using LIBRA-seq.

### CROSS-REFERENCE TO RELATED APPLICATIONS

This US application claims priority to, and the benefit of, U.S. Provisional Patent Application No. 63/584,618, filed Sep. 22, 2023, which is incorporated by reference herein in its entirety.

### STATEMENT REGARDING FEDERALLY SPONSORED RESEARCH

This invention was made with government support under Grant No. A1175245 awarded by the National Institutes of Health. The government has certain rights in the invention.

### REFERENCE TO SEQUENCE LISTING

The sequence listing submitted on Sep. 20, 2024, as an .XML file entitled “10644-177US1_ST26.xml” created on Sep. 18, 2024, and having a file size of 190,903 bytes is hereby incorporated by reference pursuant to 37 C.F.R. § 1.52 (c) (5).

### BACKGROUND

The HIV-1 Envelope protein (Env) is the sole target of neutralizing antibodies and employs several strategies to evade the immune response, including but not limited to extensive glycosylation to shield vulnerable protein epitopes, presentation of immunodominant non-neutralizing or strain-specific neutralizing epitopes, conformational masking, and vast amino acid sequence diversity. Despite these challenges, some chronically infected individuals develop broadly neutralizing serum primarily attributed to the development of broadly neutralizing antibodies (bNAbs). Efforts to recapitulate a bNAb response through vaccination have proven extremely difficult, largely due to the unique features of bNAbs including long CDRH3s, improbable mutation sets, and rare bNAb-producing B cell lineages available for activation.

Guinea pigs have proven to be an excellent model system for studying vaccine efficacy, especially in the field of HIV-1 vaccine design. Although there is a long history of using guinea pigs in vaccine studies, there is an inadequate toolkit available to study the elicited antibody response at the monoclonal level in a high-throughput and reproducible manner. Most vaccine studies with guinea pigs rely on measuring the polyclonal neutralizing response as a readout of vaccine efficacy, which can miss the nuanced contributions of rare desirable antibodies.

Currently available antibody sequencing techniques for guinea pigs rely on single-cell sorting into 96-well plates, with little information that can help down-select candidate antibodies for expression and purification.

Consistent elicitation of serum antibody responses that neutralize diverse clades of HIV-1 remains a primary goal of HIV-1 vaccine research. Little is known about the influence of the HIV immunogens and their number in vaccine cocktails. Therefore, there is a need in the art to explore bNAbs against viruses and methods to improve the vaccine efficacy.

### SUMMARY

In some aspects, disclosed herein is a method of determining broadly neutralizing antibodies in a guinea pig model system, comprising:
immunizing the guinea pig with a cocktail of two or more viral strain antigens from a virus;
isolating antigen-positive IgG-expressing B-cells from the guinea pigs immunized to the viral strain antigens using IgG positive/IgM negative flow panel;
labeling a plurality of viral strain antigens with unique antigen barcodes;
providing a plurality of barcode-labeled viral strain antigens to a population of the isolated IgG-expressing B-cells to form a mixture;
allowing the plurality of barcode-labeled viral strains to bind to the population of the isolated IgG-expressing B-cells;
washing unbound viral strain antigens from the population of isolated IgG-expressing B-cells;
separating the isolated IgG-expressing B-cells into single cell emulsions;
introducing into each single cell emulsion a unique cell barcode-labeled bead;
preparing a single cell cDNA library from the single cell emulsions;
performing nested PCR for target enrichment on the cDNA library using an inner reverse primer SEQ ID NO: 13, 14, or 15, an outer reverse primer SEQ ID NO:9, 10, 11, or 12, and a forward primer SEQ ID NO: 16 or 17;
isolating B-cell receptor (BCR) genes from the cDNA library in the single cell emulsions;
sequencing the paired heavy and light chains of the BCR;
performing PCR amplification reactions to produce a plurality of amplicons, wherein the amplicons comprise: the cell barcode and the nucleic acid sequence that identifies the particular viral strain antigens, wherein each amplicon comprises a unique molecular identifier (UMI);
sequencing the plurality of amplicons;
removing a sequence lacking the cell barcode, the UMI, or the nucleic acid sequence that identifies the particular viral strain antigens;
aligning the antibody sequence to a reference library of immunoglobulin V, D, J and C sequences;
constructing a UMI count matrix comprising the cell barcode, the nucleic acid sequence that identifies the particular viral strain antigens, and the antibody sequence;
determining a LIBRA-seq score, wherein a low score means no predicted binding to antigens and a high score means high predicted binding to antigens;
determining the nucleic acid sequence that identifies the particular viral strain antigens; and
determining broadly neutralizing antibodies binding more than two viral strain antigens.

In some embodiments, the barcode-labeled viral strain antigens are labeled with a barcode comprising a DNA sequence or an RNA sequence. In some embodiments, the cell barcode-labeled beads are labeled with a barcode comprising a DNA sequence or an RNA sequence.

In some embodiments, the antibody sequence comprises an immunoglobulin heavy chain (VDJ) sequence, or an immunoglobulin light chain (VJ) sequence.

In some embodiments, the barcode-labeled viral strain antigens comprise an antigen from a pathogen or an animal. In some embodiments, the viral strain antigens are not purified. In some embodiments, the pathogen is a virus. In some embodiments, the viral strain antigens comprise HIV-1 envelope protein (Envs)-BG505 T332N, 97ZA012.29, 286.36, 5768.04, KNH1209.18, DU172.17, HT593.1, MB539.2B7, a CD4 binding site-specific antigen (RSC3), and/or a negative control antigen (CA/09 H1). In some embodiments, the viral strain antigens from a virus comprises an antigen from human immunodeficiency virus (HIV), an antigen from influenza virus, or an antigen from respiratory syncytial virus (RSV). In some embodiments, the viral strain antigen from HIV comprises HIV-1 Env. In some embodiments, the viral strain antigen from influenza virus comprises hemagglutinin (HA). In some embodiments, the viral strain antigen from RSV comprises an RSV F protein.

In some embodiments, the plurality of viral strain antigens further comprise a panel of epitope knock-outs. In some embodiments, the plurality of viral strain antigens further comprise a panel of viral antigen variants or mutations for epitope mapping.

In some embodiments, the method of any preceding aspect further comprises determining a length of a complementarity-determining region (CDR) of the antibody specifically binding to the viral strain antigen.

In some embodiments, the method of any preceding aspect further comprises determining a motif of a CDR of the antibody specifically binding to the viral strain antigen. In some embodiments, the CDR is selected from the group consisting of CDRH3 (SEQ ID NOS: 18-47) and CDRL3 (SEQ ID NOS: 50-79).

In some aspects, disclosed herein is a method of developing a vaccine composition in a guinea pig model system, comprising:
immunizing the guinea pig with a cocktail of two or more viral strain antigens from a virus;
isolating antigen-positive IgG-expressing B-cells from the guinea pigs immunized to the viral strain antigens using IgG positive/IgM negative flow panel;
labeling a plurality of viral strain antigens with unique antigen barcodes;
providing a plurality of barcode-labeled viral strain antigens to a population of the isolated IgG-expressing B-cells to form a mixture;
allowing the plurality of barcode-labeled viral strains to bind to the population of the isolated IgG-expressing B-cells;
washing unbound viral strain antigens from the population of isolated IgG-expressing B-cells;
separating the isolated IgG-expressing B-cells into single cell emulsions;
introducing into each single cell emulsion a unique cell barcode-labeled bead;
preparing a single cell cDNA library from the single cell emulsions;
performing nested PCR for target enrichment on the cDNA library using an inner reverse primer SEQ ID NO: 13, 14, or 15, an outer reverse primer SEQ ID NO:9, 10, 11, or 12, and a forward primer SEQ ID NO: 16 or 17;
isolating B-cell receptor (BCR) genes from the cDNA library in the single cell emulsions;
sequencing the paired heavy and light chains of the BCR;
performing PCR amplification reactions to produce a plurality of amplicons, wherein the amplicons comprise: the cell barcode and the nucleic acid sequence that identifies the particular viral strain antigens, wherein each amplicon comprises a unique molecular identifier (UMI);
sequencing the plurality of amplicons;
removing a sequence lacking the cell barcode, the UMI, or the nucleic acid sequence that identifies the particular viral strain antigens;
aligning the antibody sequence to a reference library of immunoglobulin V, D, J and C sequences;
constructing a UMI count matrix comprising the cell barcode, the nucleic acid sequence that identifies the particular viral strain antigens, and the antibody sequence;
determining a LIBRA-seq score, wherein a low score means no predicted binding to antigens and a high score means high predicted binding to antigens;
determining the nucleic acid sequence that identifies the particular viral strain antigens;
determining broadly neutralizing antibodies binding more than two viral strain antigens; and
formulating a vaccine composition comprising the particular viral strain antigens that specifically bind to broadly neutralizing antibodies, wherein the particular viral strain antigens are capable of eliciting an immune response in the guinea pig.

In some embodiments, the method of any preceding aspect further comprises detecting paired heavy and light chain gene among IgG expressing B-cells from multiple guinea pigs administered with the same vaccine composition, thereby identifying vaccine composition public clonotypes.

In some embodiments, the identified vaccine composition public clonotypes are humanized.

### BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A-1D show vaccine groups were designed to evaluate the effect of including different numbers of Env strains in a cocktail immunization strategy. FIG. 1A: Vaccine groups were composed of 2 strains (Groups 1 and 2), 3 strains (Group 3), or 6 strains (Group 4). Group 1 consisted of 2 strains that have traditionally been used in the past. Envs in groups 2-4 were chosen to primarily assess the effect of having different numbers of Env strains included in the vaccine and were chosen by maximizing Env amino acid sequence diversity, as well as selecting for strains with intact glycan shields and high sensitivity to all major bNAb classes. FIG. 1B: Glycan occupancy at conserved glycosylation positions for each group is shown. Tan boxes indicate that all strains in a given group have a PNGS at the specified position. Pink boxes indicate a glycan hole, with the number denoting how many strains in the group are missing the specified glycan. Blue boxes indicate a glycan hole that is compensated for by a neighboring glycan, with the number denoting how many strains in the group have the compensatory glycan. Boxes with two colors indicate the presence of both missing and compensatory glycans at the specified position. FIG. 1C: Vaccine strains are shown with the minimum IC₅₀ values for each major bNAb class. Antibodies were grouped into bNAb classes based on epitopes targeted and other similarities such as angle of approach. FIG. 1D: Guinea pigs were immunized with 100 μg total SOSIP.DS.644.sc immunogen at 3 intervals 4 weeks apart. Final bleeds and splenocytes were collected 4 weeks after the final dose.

FIGS. 2A-2C show immunogen production and validation. FIG. 2A: Immunogens displayed expected size on SDS-PAGE. FIG. 2B: Immunogens displayed expected FPLC SEC profiles. Dotted lines indicate fractions collected indicative of trimers. FIG. 2C: Antigenicity of the immunogens was as expected measured by ELISA using a panel of standard antibodies including a CD4 binding site antibody (3BNC117), a quaternary-specific antibody (PGDM1400), as well as antibodies that recognize the open form of the trimer (F105 and 447-52d).

FIGS. 3A-3B show serum ELISA binding and effector functions. FIG. 3A: Sera from all timepoints were tested for binding against all strains of Env antigen in the vaccine regimen in an ELISA format. Final bleeds from all groups showed high binding to all Envs tested, whereas pre-bleed samples showed no binding. FIG. 3B: Sera from all groups showed measurable ADCP responses to two strains of Env antigen. Fluorescent beads with immobilized streptavidin were coated with biotinylated Env trimer and subsequently mixed with sera from each guinea pig. THP-1 monocytes were then added and ADCP was quantified as the internalization or engulfment of the antigen coated beads by THP-1 cells measured by flow cytometry. This internalization is indicated as an ADCP score. All groups were compared using a Kruskal-Wallis test and did not reach statistical significance for either antigen.

FIGS. 4A-4B show serum neutralization data for vaccine groups. FIG. 4A: Guinea pig sera was tested for neutralization against tier 2 HIV-1 pseudoviruses displaying Envs from vaccine strains (a-h) and the standard global panel (i-s) in the TZM-bl cell neutralization assay. Each row displays serum neutralization data from one guinea pig and each column represents neutralization data against a given strain of pseudovirus. ID₅₀ values are reported as colors ranging from no neutralization detected (blue) to high potency neutralization (dark red). Detectable responses per animal were compared by a two-sided Wilcoxon test. There was a statistically significant difference between groups 2 and 3 (p=0.006), with group 3 outperforming group 2. Groups 3 and 4 were comparable but trended towards group 3 having a broader median response (p=0.2). Groups 2 and 4 were comparable (p=0.6). There was a statistically significant difference between groups 1 and 3 (p=0.004), with group 3 outperforming group 1. Raw ID₅₀ values were subtracted from the ID₅₀ values for the HIV-1 specificity control, murine leukemia virus. Responses that were equal to or greater than 20 after subtraction were considered positive. Arrows denote guinea pigs 109 (top) and 112 (bottom), from which splenocytes were used for monoclonal antibody isolation. FIG. 4B: Comparison of breadth between groups as a function of the proportion of guinea pigs in each group that exhibit greater than or equal to that breadth denoted on the x-axis. The median, maximum, and minimum number of responses per group are shown to the right.

FIGS. 5A-5B show development of LIBRA-seq with guinea pig B cells. FIG. 5A: A flow panel was designed to isolate antigen-positive guinea pig B cells from splenocytes obtained from guinea pigs 109 and 112, chosen based on their broad serum neutralization profiles. Lymphocytes were gated out based on size, followed by live cells. Cells with high IgG expression and negative for IgM expression were then gated followed by antigen positive B cells. FIG. 5B: The LIBRA-seq (LInking B cell Receptor to Antigen specificity through sequencing) workflow was adapted for use in guinea pigs by modifying BCR sequencing steps. Primers shown in red indicate guinea pig-specific reverse primers that anneal to the constant regions of the guinea pig IgG genes. These custom reverse primers were mixed with existing forward primers specific to sequences appended during single cell sequencing using the Chromium Next GEM Single Cell V(D)J Reagent Kit. Following target enrichment, single cell sequencing was carried out as described in methods. The LIBRA-seq antigen screening panel is shown to the right and consists of 5 Envs, the RSC3 antigen, and an influenza HA CA/09 (negative control). Sequences of the primers used in guinea pig LIBRA-seq are shown.

FIGS. 6A-6C show identification and validation of tier 2 neutralizing monoclonal antibodies from vaccinated guinea pigs using LIBRA-seq. FIG. 6A: The LIBRA-seq output showing prioritized antibodies from guinea pig samples with predicted antigen specificities is shown. Each row consists of one B cell with the LIBRA-seq scores for each antigen, ranging from low (white) to high (blue), as well as ELISA area under the curve with the same color scheme. Antibodies isolated from guinea pig 109 have a 109 before their number, and antibodies from guinea pig 112 have a 112 before their number. FIG. 6B: Six neutralizing antibodies were identified from the positive hits. The virus strain is indicated by the title for each curve. FIG. 6C: Neutralizing antibodies were tested against the vaccine panel and 9-strain global panel. IC₅₀ values are displayed in μg/mL. Bolded numbers indicate neutralization was detected.

FIGS. 7A-7B show guinea pig antibody gene usage and public clonotype analysis. FIG. 7A: Antibody sequences were aligned to published guinea pig V genes. CDR3 sequences are shown along with CDR3 lengths. FIG. 7B: Sequence analysis revealed a guinea pig public clonotype. Antibodies 109-5 (from guinea pig 109) along with antibodies 112-22 and 112-23 (from guinea pig 112) share the same variable and joining gene usage and have >70% CDR3 identity. Antibodies 112-7, 112-8, and 112-9 aligned to their inferred variable and joining germline genes are shown.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.” “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human. In some embodiments, the subject is a guinea pig.

“Nucleotide,” “nucleoside,” “nucleotide residue,” and “nucleoside residue,” as used herein, can mean a deoxyribonucleotide or ribonucleotide residue, or other similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

The term “polynucleotide” refers to a single or double stranded polymer composed of nucleotide monomers.

The method and the system disclosed here including the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

As used herein, the term “antigen” refers to a molecule that is capable of stimulating an immune response such as by production of antibodies specific for the antigen. Antigens of the present invention can be, for example, an antigen from human immunodeficiency virus (HIV), an antigen from influenza virus, or an antigen from respiratory syncytial virus (RSV). Antigens of the present invention can also be, for example, a human antigen (e.g. an oncogene-encoded protein).

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to specifically interact with the HIV virus, such that the HIV viral infection is prevented, inhibited, reduced, or delayed. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes, heavy-chain gene and light-chain gene. The heavy-chain gene is constructed through somatic recombination of V, D, and J gene segments. In human, there are 51 VH, 27 DH, 6 JH, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called α, δ, ε, γ, and μ, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F (ab′) 2, Fab′, Fab, Fv, sFv, scFv and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)₂, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific, in another example, an antibody that specifically binds to an antigen may also bind to different allelic forms of the antigen, However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

### Methods

In some aspects, disclosed herein is a method of determining broadly neutralizing antibodies in a guinea pig model system, comprising:
immunizing the guinea pig with a cocktail of two or more viral strain antigens from a virus;
isolating antigen-positive IgG-expressing B-cells from the guinea pigs immunized to the viral strain antigens using IgG^{positive}/IgM^{negative} flow panel;
labeling a plurality of viral strain antigens with unique antigen barcodes;
providing a plurality of barcode-labeled viral strain antigens to a population of the isolated IgG-expressing B-cells to form a mixture;
allowing the plurality of barcode-labeled viral strains to bind to the population of the isolated IgG-expressing B-cells;
washing unbound viral strain antigens from the population of isolated IgG-expressing B-cells;
separating the isolated IgG-expressing B-cells into single cell emulsions;
introducing into each single cell emulsion a unique cell barcode-labeled bead;
preparing a single cell cDNA library from the single cell emulsions;
performing nested PCR for target enrichment on the cDNA library using an inner reverse primer SEQ ID NO: 13, 14, or 15, an outer reverse primer SEQ ID NO:9, 10, 11, or 12, and a forward primer SEQ ID NO: 16 or 17;
isolating B-cell receptor (BCR) genes from the cDNA library in the single cell emulsions;
sequencing the paired heavy and light chains of the BCR;
performing PCR amplification reactions to produce a plurality of amplicons, wherein the amplicons comprise: the cell barcode and the nucleic acid sequence that identifies the particular viral strain antigens, wherein each amplicon comprises a unique molecular identifier (UMI);
sequencing the plurality of amplicons;
removing a sequence lacking the cell barcode, the UMI, or the nucleic acid sequence that identifies the particular viral strain antigens;
aligning the antibody sequence to a reference library of immunoglobulin V, D, J and C sequences;
constructing a UMI count matrix comprising the cell barcode, the nucleic acid sequence that identifies the particular viral strain antigens, and the antibody sequence;
determining a LIBRA-seq score, wherein a low score means no predicted binding to antigens and a high score means high predicted binding to antigens;
determining the nucleic acid sequence that identifies the particular viral strain antigens; and
determining broadly neutralizing antibodies binding more than two viral strain antigens.

In some embodiments, disclosed herein is a method of determining broadly neutralizing antibodies in a guinea pig model system, comprising:
immunizing the guinea pig with a cocktail of two or more viral strain antigens from a virus;
isolating antigen-positive IgG-expressing B-cells from the guinea pigs immunized to the viral strain antigens using IgG positive/IgM negative flow panel;
labeling a plurality of viral strain antigens with unique antigen barcodes;
providing a plurality of barcode-labeled viral strain antigens to a population of the isolated IgG-expressing B-cells to form a mixture;
allowing the plurality of barcode-labeled viral strains to bind to the population of the isolated IgG-expressing B-cells;
washing unbound viral strain antigens from the population of isolated IgG-expressing B-cells;
separating the isolated IgG-expressing B-cells into single cell emulsions;
introducing into each single cell emulsion a unique cell barcode-labeled bead;
preparing a single cell cDNA library from the single cell emulsions;
performing nested PCR for target enrichment on the cDNA library using an inner reverse primer, an outer reverse primer, an inner forward primer, or an outer forward primer;
isolating B-cell receptor (BCR) genes from the cDNA library in the single cell emulsions;
sequencing the paired heavy and light chains of the BCR;
performing PCR amplification reactions to produce a plurality of amplicons, wherein the amplicons comprise: the cell barcode and the nucleic acid sequence that identifies the particular viral strain antigens, wherein each amplicon comprises a unique molecular identifier (UMI);
sequencing the plurality of amplicons;
removing a sequence lacking the cell barcode, the UMI, or the nucleic acid sequence that identifies the particular viral strain antigens;
aligning the antibody sequence to a reference library of immunoglobulin V, D, J and C sequences;
constructing a UMI count matrix comprising the cell barcode, the nucleic acid sequence that identifies the particular viral strain antigens, and the antibody sequence;
determining a LIBRA-seq score, wherein a low score means no predicted binding to antigens and a high score means high predicted binding to antigens;
determining the nucleic acid sequence that identifies the particular viral strain antigens; and
determining broadly neutralizing antibodies binding more than two viral strain antigens.

In some embodiments, the method of any preceding aspect further comprises determining a length of a complementarity-determining region (CDR) of the antibody specifically binding to the viral strain antigen.

In some embodiments, the method of any preceding aspect further comprises determining a motif of a CDR of the antibody specifically binding to the viral strain antigen. In some embodiments, the CDR is selected from the group consisting of CDRH3 (SEQ ID NOS: 18-47) and CDRL3 (SEQ ID NOS: 50-79).

In some embodiments, the method of any preceding aspect further comprises determining a length of a complementarity-determining region (CDR) of the antibody specifically binding to the antigen. The term “complementarity determining region (CDR)” used herein refers to an amino acid sequence of an antibody variable region of a heavy chain or light chain. CDRs are necessary for antigen binding and determine the specificity of an antibody. Each variable region typically has three CDRs identified as CDR1 (CDRH1 or CDRL1, where “H” indicates the heavy chain CDR1 and “L” indicates the light chain CDR1), CDR2 (CDRH2 or CDRL2), and CDR3 (CDRH3 or CDRL3). The CDRs may provide contact residues that play a major role in the binding of antibodies to antigens or epitopes. Four framework regions, which have more highly conserved amino acid sequences than the CDRs, separate the CDR regions in the VH or VL.

Accordingly, in some embodiments, the method of any preceding aspect further comprises determining a motif of a CDR of the antibody specifically binding to the antigen. In some embodiments, the CDR is selected from the group consisting of CDRH1, CDRH2, CDRH3, CDRL1, CDRL2, and CDRL3.

In some embodiments, the method of any preceding aspect further comprises identification of IGHV, IGHD, IGHJ, IGKV, IGKJ, IGLV, or IGLJ genes, or combinations thereof, associated with any particular combination of antigen specificities.

In some embodiments, the method of any preceding aspect further comprises identification of mutations in heavy or light FW1, FW2, FW3 or FW4 associated with any particular combination of antigen specificities.

In some embodiments, the method of any preceding aspect further comprises identification of overall gene expression profiles or select up- or down-regulated genes associated with any particular combination of antigen specificities.

In some embodiments, the method of any preceding aspect further comprises identification of surface markers, via, for example, fluorescence-activated cell sorting, or oligo-conjugated antibodies associated with any particular combination of antigen specificities

In some embodiments, the method of any preceding aspect further comprises identification of any combination of BCR sequence feature (for example, immunoglobulin gene, sequence motif, or CDR length), gene expression profile, or surface marker profile associated with any particular combination of viral strain antigen specificities.

In some embodiments, the method of any preceding aspect further comprises training a machine learning algorithm on sequence features, sequence motifs, or encoded sequence properties (such as via Kidera factors), associated with any particular combination of viral strain antigen specificities for subsequent application to sequenced antibodies lacking viral strain antigen specificity information due to not using LIBRA-seq or otherwise.

In some embodiments, the barcode-labeled viral strain antigens are labeled with a barcode comprising a DNA sequence or an RNA sequence. In some embodiments, the cell barcode-labeled beads are labeled with a barcode comprising a DNA sequence or an RNA sequence.

It should be understood that the barcode described above is conjugated to the barcode-labeled viral strain antigen in a way that are known to one of ordinary skill in the art. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553 6556), cholic acid (Manoharan et al., Bioorg. Med. Chem. Let., 1994, 4, 1053 1060), a thioether, e.g., hexyl S tritylthiol (Manoharan et al., Ann. N.Y. Acad. Sci., 1992, 660, 306 309; Manoharan et al., Bioorg. Med. Chem. Let., 1993, 3, 2765 2770), a thiocholesterol (Oberhauser et al., Nucl. Acids Res., 1992, 20, 533 538), an aliphatic chain, e.g., dodecandiol or undecyl residues (Saison Bchmoaras et al., EMBO J., 1991, 10, 1111 1118; Kabanov et al., FEBS Lett., 1990, 259, 327 330; Svinarchuk et al., Biochimie, 1993, 75, 49 54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651 3654; Shea et al., Nucl. Acids Res., 1990, 18, 3777 3783), a polyamine or a polyethylene glycol chain (Manoharan et al., Nucleosides & Nucleotides, 1995, 14, 969 973), or adamantane acetic acid (Manoharan et al., Tetrahedron Lett., 1995, 36, 3651 3654), a palmityl moiety (Mishra et al., Biochim. Biophys. Acta, 1995, 1264, 229 237), or an octadecylamine or hexylamino carbonyl oxycholesterol moiety (Crooke et al., J. Pharmacol. Exp. Ther., 1996, 277, 923 937. An oligonucleotide barcode can also be conjugated to an antigen using the Solulink Protein-Oligonucleotide Conjugation Kit (TriLink cat no. S-9011) according to manufacturer's instructions. Briefly, the oligo and protein are desalted, and then the amino-oligo is modified with the 4FB crosslinker, and the biotinylated antigen protein is modified with S-HyNic. Then, the 4FB-oligo and the HyNic-antigen are mixed together. This causes a stable bond to form between the protein and the oligonucleotide. In some embodiments, the cell barcode-labeled beads are labeled with a barcode comprising a DNA sequence or an RNA sequence. In some embodiments, the cell barcode-labeled beads are labeled with a barcode comprising a DNA sequence. In some embodiments, the cell barcode-labeled beads are labeled with a barcode comprising an RNA sequence. In some embodiments, the cell barcode-labeled beads are labeled with a barcode on the inside of the bead. In some embodiments, the cell barcode-labeled beads are labeled with a barcode encapsulated within the bead. In some embodiments, the cell barcode-labeled beads are labeled with a barcode on the outside of the bead.

As used herein, “beads” is not limited to a specific type of bead. Rather, a large number of beads are available and are known to one of ordinary skill in the art. A suitable bead may be selected on the basis of the desired end use and suitability for various protocols. In some embodiments, the bead is or comprises a particle or a bead. In some embodiments, the solid support bead is magnetic. Beads comprise particles have been described in the prior art in, for example, U.S. Pat. Nos. 5,084,169, 5,079,155, U.S. Pat. No. 473,231, and U.S. Pat. No. 8,110,351. The particle or bead size can be optimized for binding B cell in a single cell emulsion and optimized for the subsequent PCR reaction.

These oligos, which contain the cell barcode, both: (1) enable amplification of cellular mRNA transcripts through the template switch oligo that is part of the oligo containing the cell barcode, and (2) directly anneal to the antigen barcode-containing oligos from the antigen. In some embodiments, the oligos delivered from the beads have the general structure: P5_PCR_handle-Cell_barcode-UMI-Template_switch_oligo.

It is noted above that the antibody is determined as specifically binding an antigen if the LIBRA-seq score of the antibody for the antigen is increased in comparison to a control sample. It should be understood herein that between the minimum (y-axis, top) and maximum (y-axis, bottom) LIBRA-seq score for each antigen, the ability of each of 100 cutoffs was tested for its ability to classify each antibody as antigen positive or negative, where antigen positive is defined as having a LIBRA-seq score greater than or equal to the cutoff being evaluated and antigen negative is defined as having a LIBRA-seq score below the cutoff.

In some embodiments, the antibody sequence comprises an immunoglobulin heavy chain (VDJ) sequence, or an immunoglobulin light chain (VJ) sequence. In some embodiments, the antibody sequence comprises an immunoglobulin heavy chain (VDJ) sequence. In some embodiments, the antibody sequence comprises an immunoglobulin light chain (VJ) sequence.

In some embodiments, the barcode-labeled viral strain antigens comprise an antigen from a pathogen or an animal. In some embodiments, the pathogen is a virus. In some embodiments, the viral strain antigens comprise HIV-1 envelope protein (Envs)-BG505 T332N, 97ZA012.29, 286.36, 5768.04, KNH1209.18, DU172.17, HT593.1, MB539.2B7, a CD4 binding site-specific antigen (RSC3), and/or a negative control antigen (CA/09 H1).

In some embodiments, the barcode-labeled viral strain antigens comprise an antigen from an animal. In some embodiments, the animal is a mammal, including, but not limited to, primates (e.g., humans and nonhuman primates), cows, sheep, goats, horses, dogs, cats, rabbits, rats, mice and the like. In some embodiments, the subject is a human.

In some embodiments, the viral strain antigens are not purified. In some embodiments, the antigens are not purified before labeling with barcodes. Therefore, in some embodiments, the LIBRA-seq (LInking B cell Receptor to Antigen specificity through sequencing) disclosed herein can be used for soluble antigens that are not purified. Antigens can be arranged in a plate format for microexpression. Each antigen is expressed in microculture in a single well on a plate. In some embodiments, a unique barcode is added to each well, barcoded antigens are mixed together, mixed with B cells, and LIBRA-seq is performed as described herein.

In some embodiments, the LIBRA-seq disclosed herein can be used for viral strain antigens that are not in soluble form. In some embodiments, a whole virus is tagged with one or more barcodes. In some embodiments, a whole virus can use scRNA-seq to exploit the intrinsic diversity within variants of the same virus (e.g., different strains of HIV-1). In some embodiments, a pseudovirus can contain internal barcode for LIBRA-seq. In some embodiments, comprehensive mutant virus libraries can be generated using the methods of (e.g. Dingens et al (2019). An Antigenic Atlas of HIV-1 Escape from Broadly Neutralizing Antibodies Distinguishes Functional and Structural Epitopes. Immunity, 50 (2): 520-532.c3). In some embodiments, a whole cell can be tagged by lentiviral transfection with barcode or CRISPR-based tagging. In some embodiments, a membrane-anchored antigen (in particular, those for membrane proteins), e.g., the one on liposomes is tagged with one or more barcodes.

Antigens can also be formatted into antigen microarrays (for example, VirScan technology, as described in Xu G J, et al. Comprehensive serological profiling of human populations using a synthetic human virome. Science. 2015; 348 (6239): aaa0698). DNA microarrays can be used followed by phage display of antigens. In some embodiments herein, a unique barcode is added to each antigen in the microarray.

Antigens of the present invention can also be an antigen from a pathogen or an animal. In some embodiments, the antigen is from a human. In some embodiments, the antigen from a human is an antigen encoded by an oncogene. In some embodiments, the antigen encoded by an oncogene can be, for example, NY-ESO-1, MAGEA-A3, hTERT, Tyrosinase, gp100, MART-1, melanA, beta-catenin, CDC27, hsp70, HLA-A2-R170J, CEA, AFP, PSA, EBV-EBNA, HPV16-E7, MUC-1, HER-2/neu, or Mammaglobin-A.

In some embodiments, the viral strain antigen from a virus comprises an antigen from human immunodeficiency virus (HIV). In some embodiments, the viral strain antigen from a virus comprises an antigen from influenza virus. In some embodiments, the viral strain antigen from a virus comprises an antigen from respiratory syncytial virus (RSV).

In some embodiments, the viral strain antigen from HIV comprises an antigen from HIV-1. In some embodiments, the viral strain antigen from HIV comprises an antigen from HIV-2. In some embodiments, the viral strain antigen from HIV comprises HIV-1 Env. In some embodiments, the viral strain antigen from influenza virus comprises hemagglutinin (HA). In some embodiments, the viral strain antigen from RSV comprises an RSV F protein.

In some embodiments, the population of B-cells comprise a memory B-cell, a plasma cell, an IgG expressing B cell, an IgM expressing B cell, a naïve B cell, an activated B-cell, or a B-cell line. In some embodiments, the population of B-cells comprise a memory B-cell, a plasma cell, a naïve B cell, an activated B-cell, or a B-cell line. In some embodiments, the population of B-cells comprise a plasma cell. In some embodiments, the population of B-cells comprise a naïve B cell. In some embodiments, the population of B-cells comprise an activated B-cell. In some embodiments, the population of B-cells comprise a B-cell line.

In some embodiments, the amplicon comprising the cell barcode and nucleic acid sequence that identifies a particular antigen and the amplicon comprising the cell barcode and an antibody sequence are separate amplicons. In some embodiments, the antibody sequence can be a heavy chain sequence and/or a light chain sequence. In some embodiments, the antibody sequence is a heavy chain sequence. In some embodiments, the antibody sequence is a light chain sequence.

In some embodiments, the plurality of viral strain antigens further comprise a panel of epitope knock-outs. In some embodiments, the plurality of viral strain antigens further comprise a panel of viral antigen variants or mutations for epitope mapping.

In some embodiments, the nucleic acid sequence that identifies a particular antigen is a coding nucleic acid. In some embodiments, the nucleic acid sequence that identifies a particular antigen is a coding nucleic acid sequence that is covalently linked to the antigen. In some embodiments, the nucleic acid sequence that identifies a particular antigen is a nucleic acid sequence from the DNA (for example, viral DNA). In some embodiments, the nucleic acid sequence that identifies a particular antigen is an exon sequence. In some embodiments, the nucleic acid sequence that identifies a particular antigen is a transcript sequence. In some embodiments, the nucleic acid sequence that identifies a particular antigen is an antigen barcode.

In some aspects, disclosed herein is a method of developing a vaccine composition in a guinea pig model system, comprising:
immunizing the guinea pig with a cocktail of two or more viral strain antigens from a virus;
isolating antigen-positive IgG-expressing B-cells from the guinea pigs immunized to the viral strain antigens using IgG positive/IgM negative flow panel;
labeling a plurality of viral strain antigens with unique antigen barcodes;
providing a plurality of barcode-labeled viral strain antigens to a population of the isolated IgG-expressing B-cells to form a mixture;
allowing the plurality of barcode-labeled viral strains to bind to the population of the isolated IgG-expressing B-cells;
washing unbound viral strain antigens from the population of isolated IgG-expressing B-cells;
separating the isolated IgG-expressing B-cells into single cell emulsions;
introducing into each single cell emulsion a unique cell barcode-labeled bead;
preparing a single cell cDNA library from the single cell emulsions;
performing nested PCR for target enrichment on the cDNA library using an inner reverse primer SEQ ID NO: 13, 14, or 15, an outer reverse primer SEQ ID NO:9, 10, 11, or 12, and a forward primer SEQ ID NO: 16 or 17;
isolating B-cell receptor (BCR) genes from the cDNA library in the single cell emulsions;
sequencing the paired heavy and light chains of the BCR;
performing PCR amplification reactions to produce a plurality of amplicons, wherein the amplicons comprise: the cell barcode and the nucleic acid sequence that identifies the particular viral strain antigens, wherein each amplicon comprises a unique molecular identifier (UMI);
sequencing the plurality of amplicons;
removing a sequence lacking the cell barcode, the UMI, or the nucleic acid sequence that identifies the particular viral strain antigens;
aligning the antibody sequence to a reference library of immunoglobulin V, D, J and C sequences;
constructing a UMI count matrix comprising the cell barcode, the nucleic acid sequence that identifies the particular viral strain antigens, and the antibody sequence;
determining a LIBRA-seq score, wherein a low score means no predicted binding to antigens and a high score means high predicted binding to antigens;
determining the nucleic acid sequence that identifies the particular viral strain antigens;
determining broadly neutralizing antibodies binding more than two viral strain antigens; and
formulating a vaccine composition comprising the particular viral strain antigens that specifically bind to broadly neutralizing antibodies, wherein the particular viral strain antigens are capable of eliciting an immune response in the guinea pig.

In some embodiments, the method of any preceding aspect further comprises detecting paired heavy and light chain gene among IgG expressing B-cells from multiple guinea pigs administered with the same vaccine composition, thereby identifying vaccine composition public clonotypes.

In some embodiments, the identified vaccine composition public clonotypes are humanized.

In some embodiments, the nested PCR is used with a custom primer set in single-cell suspensions for target enrichment steps. In some embodiments, the target enrichment step 1 consists of a mix of Outer RP sequences 1-4 and FP 1. In some embodiments, the target enrichment step 2 consists of a mix of inner RP sequences 1-3 and FP 2. In some embodiments, the nested PCR strategy is used to amplify the guinea pig IgG genes. In some embodiments, the outer RP 1 anneals to the outer constant region of the heavy chain, and inner RP 1 anneals to the inner constant region of the heavy chain. In some embodiments, the outer RP 2 and outer RP 3 anneal to the outer constant region of the lambda light chain, and inner RP 2 anneals to the inner constant region of the lambda chain. In some embodiments, the outer RP 4 anneals to the outer constant region of the kappa light chain, and inner RP 3 anneals to the inner constant region of the kappa light chain. In some embodiments, the forward primers used in this study were obtained from the user guide of Chromium Next GEM Single Cell V(D)J Reagent Kits v1.1. In some embodiments, the forward primers anneal to the Read 1 region which is appended to the 5′ end of cDNA during single cell sequencing. In some embodiments, a target capture of 10,000 B cells was used per ⅛ 10× cassette for B cells.

In some embodiments, a flow panel is designed to isolate antigen-positive guinea pig B cells from splenocytes or blood obtained from guinea pigs. In some embodiments, the flow panel lymphocytes were gated out based on size, followed by live cells. In some embodiments, cells with high IgG expression and negative for IgM expression were then gated followed by antigen positive B cells.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for.

### Example 1

Prior work has defined key features of soluble HIV-1 Envelope (Env) immunogen cocktails that influence the neutralization breadth and potency of multivalent vaccine-elicited antibody responses including the number of Env strains in the regimen. Immunization groups were designed that consisted of different numbers of SOSIP Env strains to be used in a cocktail immunization strategy: the smallest cocktail (group 2) consisted of a set of two Env strains, which were a subset of the three Env strains that made up group 3, which in turn were a subset of the six Env strains that made up group 4. Serum neutralizing titers were modestly broader in guinea pigs that were immunized with a cocktail of three Envs compared to cocktails of two and six, suggesting that multivalent Env immunization could provide a benefit but may be detrimental when the cocktail size is too large. The LIBRA-seq platform was adapted for antibody discovery to be compatible with guinea pigs, and isolated several tier 2 neutralizing monoclonal antibodies. Three antibodies isolated from two separate guinea pigs were similar in their gene usage and CDR3s, establishing evidence for a guinea pig public clonotype elicited through vaccination. Taken together, this work investigated multivalent HIV-1 Env immunization strategies and provides a novel methodology for screening guinea pig B cell receptor antigen specificity at a high throughput level using LIBRA-seq.

LIBRA-seq (linking B cell receptor to antigen specificity through sequencing) platform is compatible with guinea pigs, thus allowing for a high-throughput technology to assess the antigen specificity of guinea pig B cells against potentially large numbers of antigen variants, while also providing a prioritization metric (the LIBRA-seq Score) to down-select candidate antibodies for validation. This technology is especially beneficial for deconvoluting the antigen specificity patterns in antibody responses to multivalent vaccines (cocktails of immunogens).

Researchers have made significant progress through multivalent vaccination approaches targeted against HIV-1. Inclusion of more Env strains in a vaccine has been shown to increase the elicited neutralization breadth. More recent work systematically defined Env amino acid signatures associated with sensitivity to bNAbs and appended these amino acid motifs onto Env immunogens, termed signature-based epitope targeting (SET) immunogens. When combined with an immunogen engineered to display higher epitope diversity, SET immunogens achieved remarkable breadth in a guinea pig model. To further investigate important factors of multivalent Env immunization regimens, the effect of the number of Env strains included in cocktail immunizations was explored. Different Env cocktail sizes were computationally selected while simultaneously optimizing for bNAb neutralization sensitivity and Env amino acid sequence diversity. Env immunogens that are missing conserved glycans tend to elicit strain-specific neutralizing antibodies that target the glycan holes but lack the ability to neutralize virus isolates that have an intact glycan shield. To mitigate bias introduced by glycan holes, Env strains were selected based on the presence of potential N-linked glycosylation sites (PNGS). The guinea pigs were immunized with computationally selected cocktails of two, three, or six different Env strains expressed in the SOSIP format and evaluated the serum neutralizing antibody response at the polyclonal level. As a proof of concept, the LIBRA-seq platform was adapted to interrogate the B cell repertoire of two guinea pigs (gp109 and gp112), and several hundred B cells were isolated with B cell receptor (BCR) sequences that were predicted to bind to Env based on their LIBRA-seq scores. Thirty antibodies were selected for expression and validated binding for twenty-seven of these antibodies, of which six neutralized tier 2 pseudoviruses. Notably, three of these antibodies (two from gp112 and one from gp109) used the same germline V and J-genes and remarkably similar CDR3 regions for both the heavy and light chains, establishing the existence of vaccine-elicited guinea pig public antibody clonotypes.

### Results

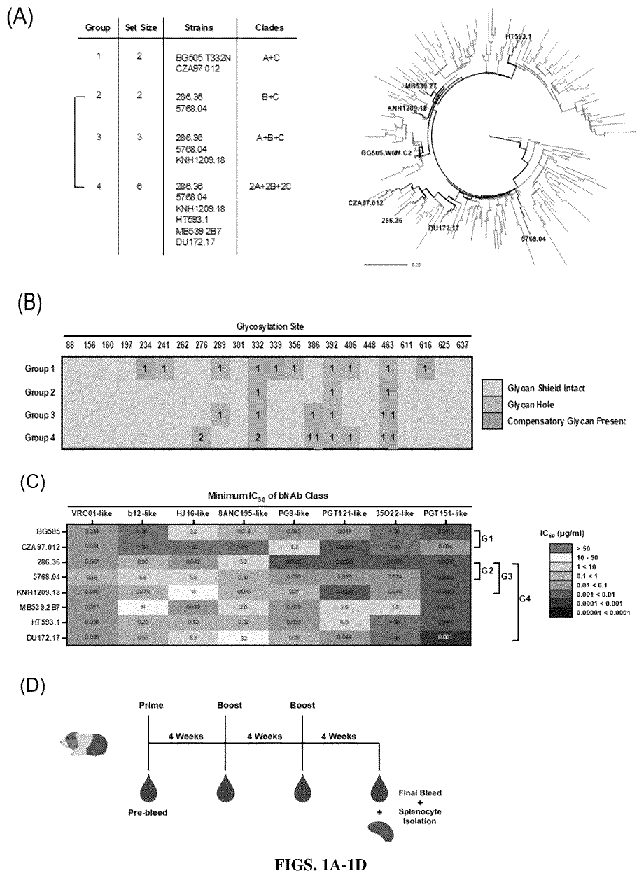

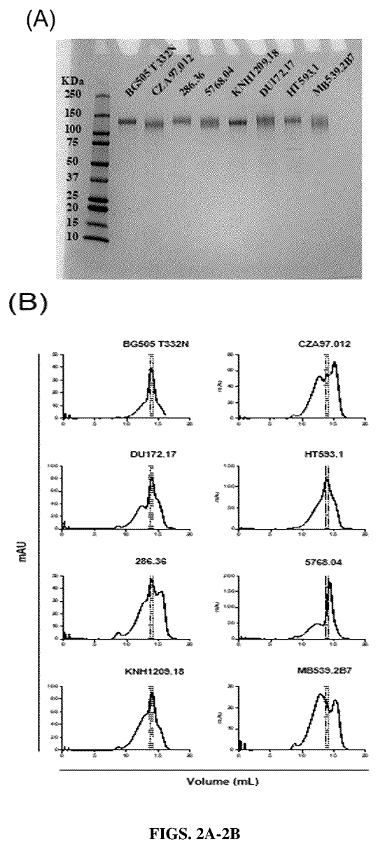

Immunization groups—The vaccine trial was composed of four groups, each of which was immunized with soluble Env immunogen containing the DS.SOSIP.664 mutation set along with a serine glycine linker replacing the furin cleavage site. Group 1 consisted of a pair of strains (BG505 T332N and CZA97.012) that in prior studies have not elicited a robust neutralizing antibody response in cocktail immunizations, serving as a baseline control. Groups 2-4 consisted of strains that were selected using an algorithm that optimized for glycan shield coverage, sensitivity to known bNAb classes, and Env sequence diversity, but for different numbers of strains in each cocktail. Group 2 consisted of two strains (5768.04 and 286.36), Group 3 consisted of three strains (5768.04, 286.36, and KNH1209.18), and Group 4 consisted of six strains (5768.04, 286.36, KNH1209.18, HT593.1, MB539.2B7, and DU172.17) (FIG. 1).

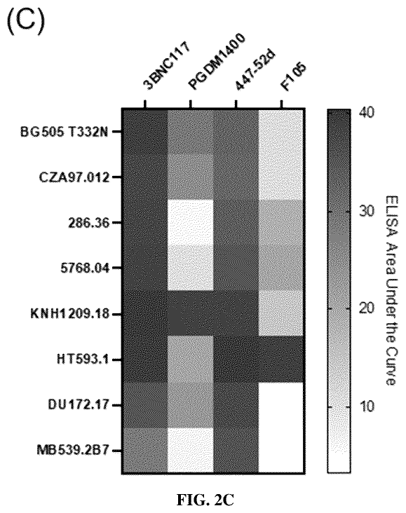

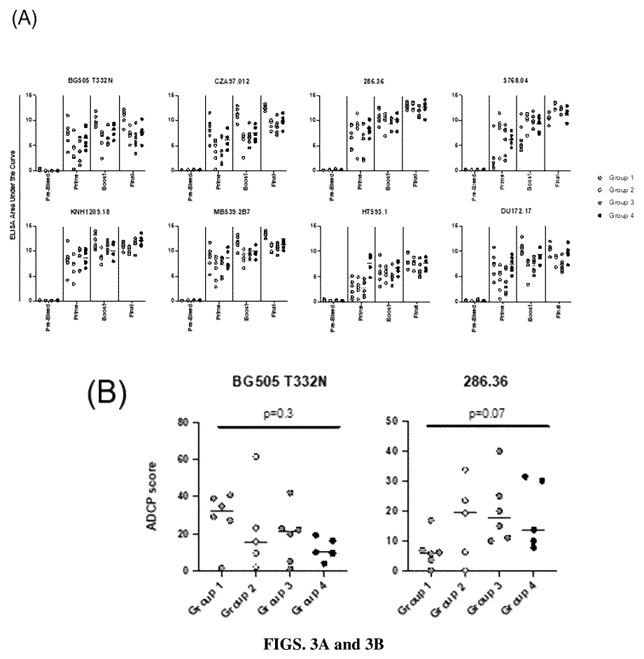

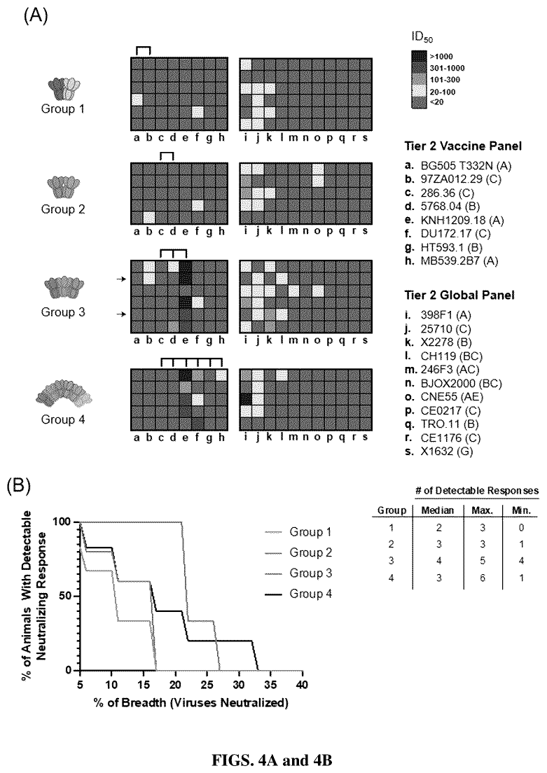

Immunogenicity of the multivalent vaccine cocktails—Guinea pigs were vaccinated intramuscularly in the quadriceps with 100 μg total Env immunogen formulated with CpG/Emulsigen at weeks 0, 4, and 8 (FIG. 1D). For each injection, half of the formulation was injected into the left quadricep, and the other half was injected into the right quadricep. Serum was collected at each time point prior to injections. Guinea pigs were exsanguinated 4 weeks after the final injection, and splenocytes were isolated for downstream antigen-specific B cell sorting. The size and antigenicity of the Env immunogens were verified with SDS-PAGE, FPLC, and ELISA binding to a panel of HIV-1 antibodies (FIG. 2). All groups showed a robust antibody response towards autologous and heterologous strains of Env trimer by ELISA (FIG. 3A). In addition to serum antibody binding, the ability of antibodies in the serum to mediate antibody-dependent cellular phagocytosis (ADCP) was evaluated in a bead-based assay and found that serum from all groups exerted ADCP to autologous and heterologous antigens to varying degrees (FIG. 3B). Next, the neutralizing antibody response was evaluated using serum from the final timepoints against a large panel of HIV-1 pseudoviruses that included eight vaccine strains and eleven global panel strains (FIG. 4A). Overall, group 3 exhibited slightly broader levels of serum neutralization with a median number of detectable responses against 4 pseudoviruses (21% breadth) compared to a range of 2.5-3 (13-16% breadth) for the other groups. Notably, all animals in group 3 had detectable neutralizing responses against at least 21% of the full virus panel, and 2/6 animals neutralized 26% of the viruses. In comparison, none of the animals in group 2 and only 2/5 animals in group 4 showed neutralizing responses against at least 21% of the viruses (FIG. 4B). These data suggest that increasing the number of diverse strains in a cocktail regimen has the potential to expand neutralization breadth, but that adding more strains may not always be beneficial.

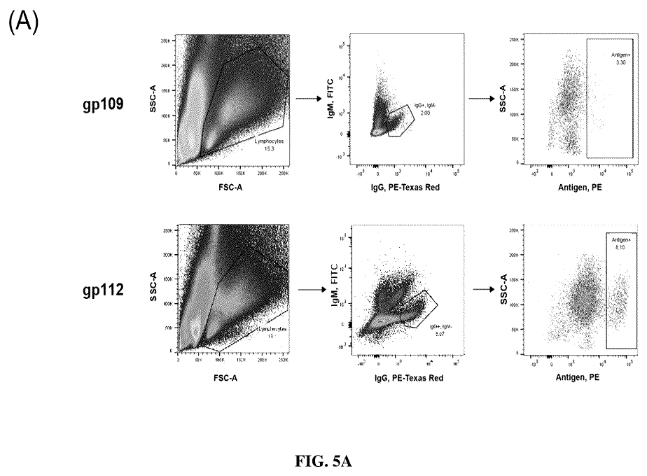

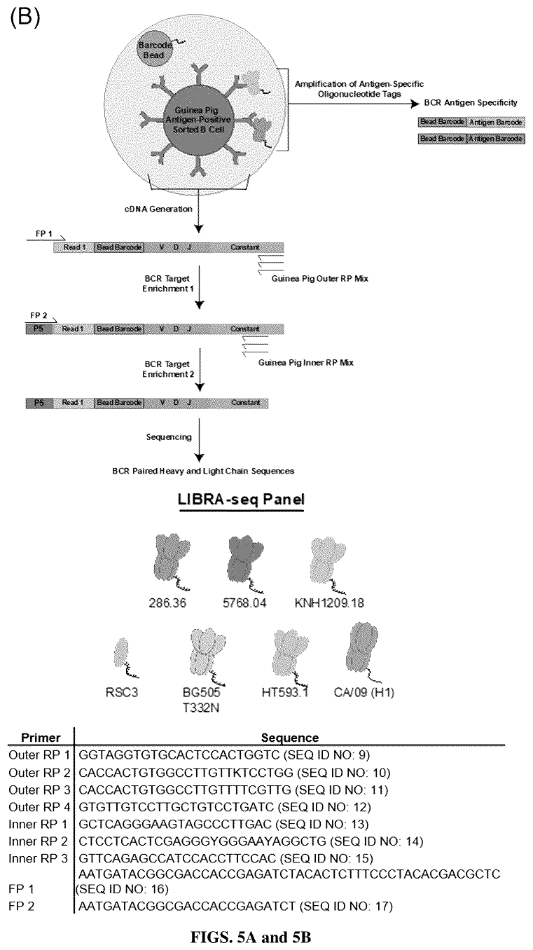

Isolation and characterization of tier 2 neutralizing monoclonal guinea pig antibodies with LIBRA-seq. LIBRA-seq was adapted for use with guinea pig splenocytes to isolate tier 2 neutralizing antigen-specific monoclonal antibodies from gp109 and gp112 (indicated with arrows in FIG. 4A). LIBRA-seq is a single-cell sequencing technology for high-throughput screening of B cells against a diverse antigen panel labeled with oligonucleotide barcodes. A flow panel was designed to isolate antigen-positive IgG-expressing B cells. Later, IgG^{Positive} IgM^{Negative} cells were selected to bias towards IgG class-switched B cells, followed by isolation of antigen-positive B cells (FIG. 5A). The LIBRA-seq antigen panel consisted of 3 autologous Envs (286.36, 5768.04, and KNH1209.18) that were part of the group 3 immunization cocktail, 2 heterologous Envs (HT593.1 and BG505 T332N), a gp120-based CD4 binding site-specific antigen (RSC3), and a negative control antigen (CA/09 H1), all of which were biotinylated and conjugated with distinct oligonucleotide barcodes to allow for staining with PE-streptavidin and antigen identification through sequencing (FIG. 5B). A critical step in single cell sequencing to obtain paired heavy and light BCR chains is target enrichment following cDNA generation. The inner and outer reverse primers were generated based on previous work with antigen-specific B cell sorting with guinea pig samples, whereas the forward primers are not species-specific, and thus did not require alteration (FIG. 5B).

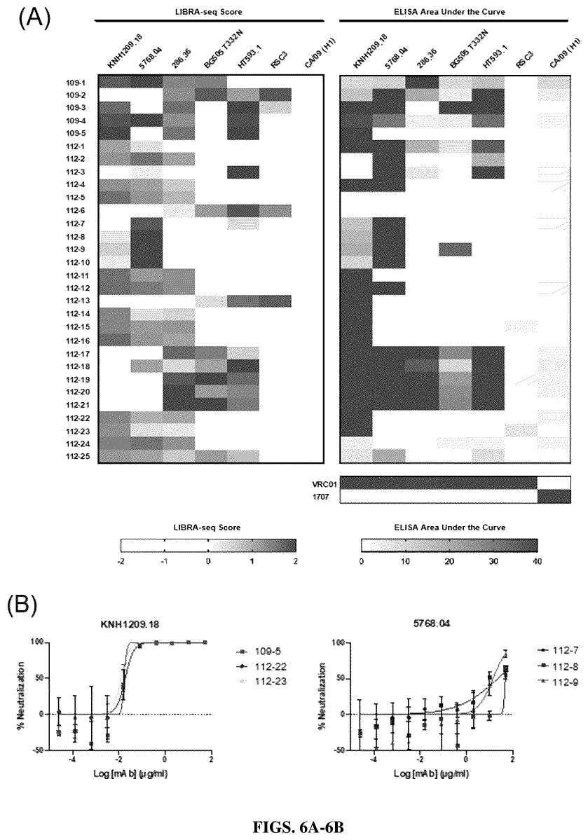

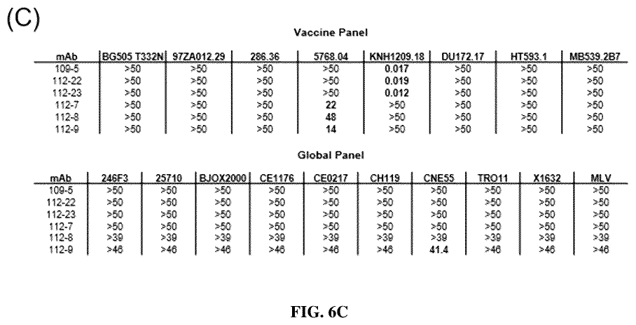

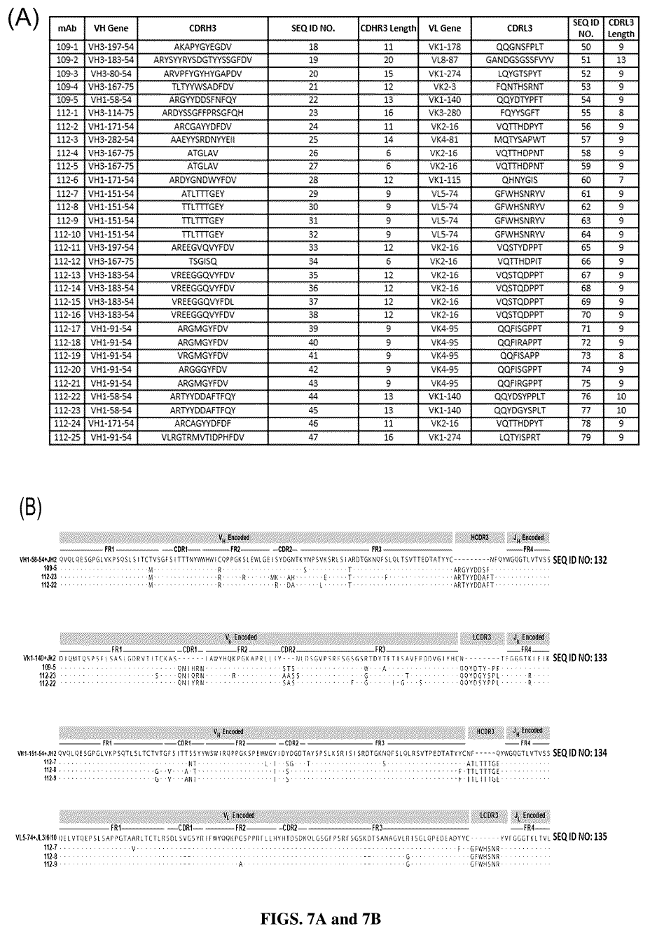

From the LIBRA-seq output a total of 277 cells were obtained from gp109, and 108 cells from gp112. From these thirty paired heavy and light chain sequences were selected to express as humanized IgG1 antibodies (five from gp109 and twenty-five from gp112) based on antigen specificity scores for Envs in the LIBRA-seq panel (FIG. 6A). Of the thirty antibodies, twenty-seven are bound to at least one Env antigen in ELISA, and several are bound to multiple diverse strains. In total, 90% of the LIBRA-seq-identified cells were confirmed to be HIV-specific. Next, the antibodies were selected for neutralization against a panel of autologous strains of tier 2 HIV-1 pseudoviruses (286.36, 5768.04, and KNH1209.18) in the TZM-bl cell neutralization assay. A total of six antibodies were identified as neutralizing, with three potently neutralizing the KNH1209.18 strain and three neutralizing the 5768.04 strain with low potency (FIG. 6B). The six neutralizing antibodies were found to be strain specific as they did not neutralize any other strains in the vaccine panel nor a global panel, except for antibody 112-9 which showed weak neutralization against CNE55 (FIG. 6C). V-gene usage as well as CDR3 sequence and length for all thirty antibodies are reported (FIG. 7A). Sequence comparison of the three antibodies that neutralized the KNH1209.18 strain (two from gp112 and one from gp109) revealed the use of the same germline genes and high CDR3 identity for both the heavy and light chains, suggesting a vaccine-elicited public guinea pig clonotype (FIG. 7B).

### DISCUSSION

It was investigated the effect of the number of Env strains included in a multivalent HIV-1 vaccine on the elicited serum neutralization breadth in guinea pigs. In parallel, LIBRA-seq platform was adapted to be compatible with guinea pig vaccine samples and this technology was applied to the vaccine study described here, as a proof of concept, thus expanding the toolkit for studying vaccine-elicited antibody responses at the monoclonal level. The Env strains used in the immunization cocktails were selected using criteria that have been shown to be critical in eliciting broadly neutralizing antibody responses, including glycan shield coverage, bNAb neutralization sensitivity, and Env amino acid sequence diversity. Immunization group 3, immunized with a cocktail of three Envs, exhibited the broadest neutralizing serum of all the groups. This could suggest that in the context of multivalent HIV-1 vaccines, larger cocktail sizes are not always better, as was evidenced with the results for group 4, immunized with a cocktail of six Envs that included all Envs from group 3 along with three additional diverse Envs. The modest breadth and potency of the elicited neutralizing antibody responses in this study, combined with the small samples sizes in each group, make definitive conclusions about the effect of strain number on multivalent HIV-1 vaccination out of reach. However, the trends observed in this study justify further investigation into these tunable features of multivalent vaccination.

It is interesting, however, to further dissect the differences in neutralizing serum responses for the three immunization groups, given that the Env strains in group 2 are a subset of the Env strains in group 3, which in turn are a subset of the Env strains in group 4. None of the animals from group 2 showed neutralization against the two autologous viruses (286.36 and 5768.04). In contrast, two animals from group 3 (that also included KNH1209.18 in addition to the other two viruses from group 2) were able to neutralize 5768.04, with one of the animals reaching high potency (ID₅₀ of >250). These observations suggest that the addition of an additional strain can modulate the immune response toward neutralization against an autologous strain for which neutralization was otherwise not observed. Compared to group 4, a larger number of animals from group 3 were capable of neutralizing higher fractions of the tested viruses. While this is a single example with limited data, it is also interesting to note that adding more strains to multivalent vaccines does not necessarily result in improved neutralization breadth, compared to groups with smaller sizes (group 3 was better than both the group 4 and group 2). Together, these results indicate that the selection of strains for incorporation into multivalent vaccines can indeed impact neutralization breadth. The results with the group 3 set are the most promising among the four multivalent vaccines tested, including the elicitation of autologous neutralization against all strains that are part of the multivalent vaccine, consistent neutralization breadth among all animals, and increased overall potency against heterologous viruses.

A major push in the field of HIV-1 vaccine design centers on the use of soluble Env immunogens that faithfully mimic the native closed prefusion conformation of Env found on live virions. The closed conformation was recapitulated with the immunogens using the SOSIP mutation sets, all immunogens in this study exhibited some degree of binding in ELISA to antibodies known to target more open conformations of Env (antibodies 447-52d and F105). More stringent purification methods, such as affinity columns using antibodies known to preferentially bind to the closed conformation of Env, will mitigate this issue. When compared to all other immunogens in the vaccine panel, the KNH1209.18 strain showed the strongest binding to PGDM1400, an antibody that specifically binds Env in the closed conformation. Consistent elicitation of potent autologous neutralizing serum directed towards this strain was observed in groups 3 and 4 and isolated several potent neutralizing monoclonal antibodies that recapitulate this phenotype. It is possible that the KNH1209.18 strain more closely mimics a near-native state of Env in comparison to other strains used in this study, and perhaps this explains the strong elicitation of potently neutralizing antibodies directed towards this strain. Recent work has demonstrated that SOSIPs does not fold into the native conformation of Env that is found on virions. Additionally, SOSIP mutations have been found to alter several features of Env including glycosylation and antigenicity. Although SOSIPs does not represent the biologically relevant prefusion conformation of Env, several bNAbs bind to SOSIPs, thus it is still be feasible to elicit antibodies that target bNAb epitopes using SOSIP immunogens.

The ability to characterize the vaccine-elicited antibody response at the monoclonal level is paramount to understanding vaccine trials and designing next-generation vaccines. LIBRA-seq technology is compatible with guinea pig samples, which allowed to probe the antigen specificity of the guinea pig B cell repertoire at unprecedented levels. LIBRA-seq was validated on guinea pig samples using splenocytes from two guinea pigs in group 3, gp109 and gp112. A diverse panel of Env antigens was used in the LIBRA-seq screening library to help prioritize antibody leads for further characterization. Several neutralizing antibodies were identified among those tested for neutralization. Of note, for gp112, the serum neutralization assays failed to detect any neutralization against the 5768.04 strain, for which three of the monoclonal antibodies showed modest neutralization ability. This emphasizes the importance of being able to isolate and characterize antibodies at the monoclonal level, as it allows us to identify rare antibodies that may not be reflected in the signals observed with polyclonal assays. Although previous HIV-1 vaccine studies with guinea pigs have reported elicitation of polyclonal sera capable of neutralizing tier 2 pseudoviruses, most did not isolate monoclonal antibodies. In this study, reverse primers specific for the IgG guinea pig BCR genes for target enrichment during sequencing were utilized. In this study a human BCR reference library was utilized to assemble the sequence contigs obtained from single cell sequencing into full-length variable regions of the BCR genes, which likely only allowed the recovery of a subset of guinea pig BCR sequences (those that map close enough to human BCR sequences). Finally, the guinea pig antibodies in this study were expressed with human IgG1 constant regions, which alters their binding and could play a role in the concordance between LIBRA-seq scores and ELISA binding. Previous studies have shown that antibodies expressed in their native isotype can display dramatically different binding profiles compared to alternative isotype expressions. Heterologous neutralization in the serum of several guinea pigs was detected. For example, guinea pig 109 had an ID50 value of 83 against the 25710 strains. Likewise, guinea pig 112 had ID50 values of 53, 158, and 63 for the 25710, X2278, and CH119 strains respectively. It is therefore possible that heterologous neutralizing antibodies were elicited, but they did not overlap with the monoclonal antibodies that were selected for validation.

Sequence analysis of the three antibodies that potently neutralize the KNH1209.18 strain (109-5, 112-22, and 112-23) revealed the existence of vaccine-elicited public clonotypes in guinea pigs. In addition to the similar point mutations present on the heavy chains, a large insertion of 6 similar amino acids on the light chain of all three antibodies was identified (FIG. 7B). While there are similarities between the three antibodies, there were multiple residue positions with divergent mutations in the different antibodies, likely indicating that antigen-driven evolution of the antibodies occurred. Guinea pigs are a popular model for assessing vaccine responses, and that multivalent vaccine cocktails are of interest for a variety of pathogens of biomedical significance, including influenza, hepatitis C, SARS-COV-2, and HIV-1 among others. As such, the LIBRA-seq technology plays an important role as a general platform for vaccine development and evaluation.

### Materials and Methods

### HIV-1 Env Strain Selection Algorithm

Prior work has established key factors related to the characteristics of Env strains used for immunization. These include the presence of an intact glycan shield at conserved positions, the availability of bNAb epitopes, the number of strains, as well as the level of amino acid sequence diversity between the strains, included in a multivalent vaccine. A multi-objective optimization algorithm was developed that selects for groups of Env strains that are optimized for the features listed in FIG. 1. Glycan Shield Coverage: A set of ˜5,000 representative HIV-1 strains was selected from the Los Alamos National Laboratory HIV database and their Env amino acid sequences were aligned to the reference HXB2 strain. From the Env alignment, all residue positions that corresponded to an N-linked glycosylation sequon were extracted from each strain. Residue positions for which at least 50% of strains have an N-linked glycosylation sequon were defined as conserved glycan positions. For strains that were missing conserved glycans, structural analysis of the Env trimer structure was performed to identify potential compensatory glycans that are within 10 Å of a missing conserved glycan. For each strain, the list of conserved and compensatory glycans was then used for further analysis. The completeness of the glycan shield is shown for each vaccine group (FIG. 1B). Availability of bNAb Epitopes: The algorithm was designed to select Envs from HIV-1 strains that are sensitive to every major bNAb class, and thus present numerous epitopes that are capable of eliciting antibodies that evolve neutralization breadth. Published datasets of bNAb-virus neutralization were used to divide bNAbs into discrete sets of epitope specificities (e.g. VRC01-like, PG9-like, etc.). The VRC01-like bNAb group consisted of VRC01, VRCPG04, VRC-CH31, 3BNC117, 12A12, VRC-PG20, VRC-23, and VRC27. The b12-like group consisted of b12. The HJ16-like group consisted of HJ16. The 8ANC195 group consisted of 8ANC195. The PG9-like group consisted of PG9, PGT145, and CH01. The PGT121-like group consisted of PGT121, PGT128, and PGT135. The 35022-like group consisted of 35022. The PGT151-like group consisted of PGT151 and N123-VRC34.01. For each strain and bNAb specificity group, the minimum (best), median, and maximum (worst) neutralization IC₅₀ values among all bNAbs in that group were computed. Strains with minimum neutralization values of greater than 1 μg/ml for any bNAb specificity group, and strains for which two or more bNAb groups include an antibody that cannot neutralize the given strain (IC₅₀ value of >50 μg/ml) were filtered out. The remaining strains were used for further optimization. Neutralization sensitivities of each strain composing the vaccine groups for every major bNAb class are shown (FIG. 1C). Env Sequence Diversity: The Env sequence diversity within each combination of strains was computed. This was done both for the entire Env sequence (to account for overall clade diversity), as well as specifically for the protein surface residues (to account for antibody epitope diversity). The number of Env strains used in a multivalent vaccine have opposing effects: on one hand, adding more strains allow for closer mimicking of virus swarms during HIV-1 infection; on the other hand, the inclusion of more strains increase the likelihood of generating off-target antibody responses. To assess this effect, the algorithm to pick set sizes of 2, 3, or 6 Env strains was used (FIG. 1A).

### Immunogen Expression and Purification

Immunogens were designed using the SOSIP platform to create soluble Env proteins stabilized in the prefusion conformation. In addition to the SOS (A501C and T605C) and IP (1559P) mutations that form the base of the SOSIP platform, Envs were truncated at position 664 and added the DS (1201C and A433C) mutation set to prevent CD4 triggering. The furin cleavage site between gp120 and gp41 was replaced with a serine-glycine linker (length 15) to create single-chain constructs. The immunogens were produced in Expi293F cells by transient transfection in FreeStyle F17 expression media (Thermo Fisher) supplemented to a final concentration of 0.1% Pluronic Acid F-68 and 20% 4 mM L-glutamine using Expifectamine transfection reagent (Thermo Fisher Scientific), cultured for 4-7 days at 8% CO₂ saturation and 37° C. with shaking. After transfection, cultures were centrifuged at 4000 g for 20 minutes. Supernatant was filtered with Nalgene Rapid Flow Disposable Filter Units with PES membrane (0.45 μm), and then run slowly over a column with agarose-bound Galanthus nivalis lectin (Vector Laboratories cat no. AL-1243-5) at 4° C. The column was washed with PBS, and protein was eluted with 15 mL of 1M methyl-α-D-mannopyranoside. The protein elution was buffer exchanged 3 times into PBS and concentrated using 100 kDa Amicon Ultra centrifugal filter units. Concentrated protein was run on a Superose 6 Increase 10/300 GL on the AKTA FPLC system. Peaks corresponding to trimeric species were identified based on elution volume and antigenicity to quaternary structure-preferring antibody (PGDM1400) (FIG. 2).

### Immunizations

Immunizations were conducted by, and in accordance with the guidelines of, Cocalico Biologicals, Inc., Reamstown, PA, USA. Immunizations were done at four-week intervals, for a total of three injections per guinea pigs. A pre-bleed sample was taken prior to the first immunization. Guinea pigs were healthy, outbred, research-naïve, between 350-500 grams, about 1-2 months of age, half male and half female for each vaccine group. Immediately before each administration, 100 μL of poteen mix (100 μg total Env) in PBS was mixed with 325 μL of CpG at 0.154 μg/μL (IDT), then with 75 μL of Emulsigen-D (MVP Adjuvants), giving a total volume of 0.5 mL. The final mixture was vortexed 5 times with 10 second intervals, and then was administered bilaterally in the quadricep muscles with half (0.25 mL) in the left quadricep and half (0.25 mL) in the right quadricep. Bleeds were obtained from the vena cava of anesthetized animals four weeks after each immunization. Spleens were collected for splenocyte isolation four weeks after the final immunization.

### Biotinylation of Antigens

Protein antigens used for LIBRA-seq, and serum ELISA contained a C-terminal Avi-tag and were site specifically biotinylated using BirA biotin-protein ligase reaction kit (Avidity) according to manufacturer's instructions.

### Oligonucleotide Barcodes

Oligos that possess a 15 bp antigen barcode, a sequence capable of annealing to the template switch oligo that is part of the 10× bead-delivered oligos and contain truncated TruSeq small RNA read 1 sequence in the following structure: 5′-CCTTGGCACCCGAGAATTCCANNNNNNNNNNNNNNNCCCATATAAGA*A*A-3′ (SEQ ID NO: 1), where Ns represents the antigen barcode. Oligos were ordered from Sigma-Aldrich and IDT with a 5′ amino modification and HPLC purified. The following antigen barcodes were used: GCAGCGTATAAGTCA (CA/09) (SEQ ID NO:2), CAGTAAGTTCGGGAC (RSC3) (SEQ ID NO:3), TGACCTTCCTCTCCT (HT593.1) (SEQ ID NO: 4), GACCTCATTGTGAAT (KNH1209.18) (SEQ ID NO:5), TAACTCAGGGCCTAT (5768.04) (SEQ ID NO:6), CAGATGATCCACCAT (286.36) (SEQ ID NO:7), ATCGTCGAGAGCTAG (BG505 T332N) (SEQ ID NO:8).

### Conjugation of Oligonucleotide Barcodes to Antigens

For each antigen, a unique DNA barcode was directly conjugated to the antigen using a SoluLINK Protein-Oligonucleotide Conjugation kit (TriLink, S-9011) according to the manufacturer's protocol. Briefly, the oligo and protein were desalted, and then the amino-oligo was modified with the 4FB crosslinker, and the biotinylated antigen protein was modified with S-HyNic. Then, the 4FB-oligo and the HyNic-antigen were mixed together. This causes a stable bond to form between the protein and the oligonucleotide. The concentration of the antigen-oligo conjugates was determined by a BCA assay, and the HyNic molar substitution ratio of the antigen-oligo conjuagtes was analyzed using NanoDrop according to the Solulink protocol guidelines. AKTA FPLC was used to remove excess oligonucleotide from the protein-oligo conjugates, which were also checked using SDS-PAGE with a silver stain.

### Enrichment of Antigen-Specific Guinea Pig B Cells

Splenocytes from each guinea pig were sorted and sequenced on separate days. Frozen splenocytes were thawed and resuspended in 10 ml of pre-warmed RPMI 1640 medium supplemented with 10% heat-inactivated Fetal bovine serum (FBS). The cells were then washed with 10 ml of warm DPBS supplemented with 0.1% Bovine serum albumin (BSA). Cells were resuspended in DPBS-BSA and stained with cell markers including viability dye (Ghost Red 780), anti-guinea pig IgM-FITC, and anti-guinea pig IgG-Alexa Fluor 594 in the dark for 30 minutes at room temperature. Cells were then washed three times with DPBS-BSA at 300 g for 5 minutes. Antigen-oligo conjugates were then added and incubated with the cells for 30 minutes in the dark. Cells were then washed three times with DPBS-BSA at 300 g for 5 minutes. Streptavidin-PE was added to label the cells with bound antigen and incubated for 15 minutes in the dark at room temperature. Cells were then washed three times with DPBS-BSA, resuspended in DPBS-BSA, and then sorted by FACS.

### 10× Genomics Single Cell Processing and Next Generation Sequencing

Single-cell suspensions were loaded onto the Chromium Controller microfluidics device (10× Genomics) and processed using previously described methods for LIBRA-seq, with the exception that a custom primer set outlined in (FIG. 5B) was used for target enrichment steps. Target enrichment step 1 consisted of a mix of Outer RP sequences 1-4 (SEQ ID NOS: 9-12) (final concentrations of 0.5 μM each) and FP 1 (SEQ ID NO:16) (final concentration 1 μM). Target enrichment step 2 consisted of a mix of inner RP sequences 1-3 (SEQ ID NOS: 13-15) (final concentration of 0.5 μM each) and FP 2 (final concentration of 1 μM). Briefly, these sequences were designed to be used in a semi-nested PCR strategy to amplify the guinea pig IgG genes. Outer RP 1 (SEQ ID NO: 9) anneals to the constant region of the heavy chain, and inner RP 1 (SEQ ID NO: 13) anneals to the constant region of the heavy chain as well, but a more inner region. Outer RP 2 (SEQ ID NO: 10) and Outer RP 3 (SEQ ID NO: 11) anneal to the constant region of the lambda light chain, and inner RP 2 (SEQ ID NO: 14) anneals to the constant region of the lambda chain as well but at a more inner region. Likewise, Outer RP 4 (SEQ ID NO: 12) anneals to the constant region of the kappa light chain, and inner RP 3 (SEQ ID NO: 15) anneals to the constant region of the kappa light chain as well but at a more inner region. The forward primers used in this study were obtained from the user guide of Chromium Next GEM Single Cell V(D)J Reagent Kits v1.1. Briefly, the forward primers anneal to the Read 1 region which is appended to the 5′ end of cDNA during single cell sequencing. A target capture of 10,000 B cells was used per ⅛ 10× cassette for B cells. Slight modifications were made to intercept, amplify, and purify the antigen barcode libraries as previously described.

### Sequence Processing and Bioinformatics Analysis

Paired-end FASTQ files of oligonucleotide libraires were taken as input, to process and annotate reads for cell barcodes, unique molecular identifiers (UMIs) and antigen barcodes, resulting in a cell barcode-antigen barcode UMI count matrix. B cell receptor contigs were processed using CellRanger 3.1.0 (10× Genomics) and GRCh38 Human V(D)J 7.0.0 as reference, while the antigen barcode libraries were also processed using CellRanger (10× Genomics). The cell barcodes that overlapped between the two libraries formed the basis of the subsequent analysis. Cell barcodes that only had non-functional heavy chain sequences as well as cells with multiple functional heavy chain sequences and/or multiple functional light chain sequences, were eliminated, reasoning that these are multiplets. The B cell receptor contigs were aligned (filtered_contigs.fasta file output by CellRanger, 10× Genomics) to IMGT references genes using IMGT/HighV-QUEST. The output of HighV-Quest was parsed using Change-O and combined with an antigen barcode UMI count matrix. Finally, the LIBRA-seq score for each antigen in the library for every cell was determined. Raw sequencing results were aligned to the human VDJ reference genome using Cell Ranger to assemble heavy and light chains due to the absence of a suitable guinea pig VDJ reference. For guinea pig gene assignments of the antibodies shown in (FIG. 6C) and (FIG. 7A), the immunoglobulin amino acid sequences for guinea pigs were obtained and aligned to amino acid sequences of antibodies discovered in the work described here using Clustal Omega.

### Public Antibody Analysis

Public clones were identified on the basis of a minimum of 70% amino acid sequence identity in both CDRH3 and CDRL3 regions and matching heavy and light variable (V) and joining (J) gene usage.

### Humanized Guinea Pig Antibody Expression and Purification

The variable regions of guinea pig antibody sequences obtained from LIBRA-seq were appended into antibody expression vectors that contained human constant regions for heavy and light chains to create humanized guinea pig antibody sequences. Humanized guinea pig antibodies were produced in Expi293F cells by transient transfection in FreeStyle F17 expression media (Thermo Fisher) supplemented to a final concentration of 0.1% Pluronic Acid F-68 and 20% 4 mM L-glutamine using Expifectamine transfection reagent (Thermo Fisher Scientific), cultured for 4-7 days at 8% CO₂ saturation and 37° C. with shaking. After transfection, cultures were centrifuged at 4000 g for 20 minutes. Supernatant was filtered with Nalgene Rapid Flow Disposable Filter Units with PES membrane (0.45 μm), and then run slowly over a column with agarose-bound Protein A at 4° C. The column was washed with PBS, and antibody was eluted with 5 mL of 100 mM glycine at a pH of 2.8. The elution was buffer exchanged 3 times into PBS and concentrated using 50 kDa Amicon Ultra centrifugal filter units.

### Enzyme Linked Immunosorbent Assay (ELISA) for Immunogens

ELISA for immunogens utilized a lectin-based capture format. Galanthus nivalis lectin was plated at 2 μg/mL overnight at 4° C. The next day, plates were washing three times with PBS supplemented with 0.05% Tween20 (PBS-T) and blocked with 1% BSA in PBS-T. Plates were incubated at room temperature for one hour and then washed three times with PBS-T. Immunogens were diluted to 2 μg/mL in 1% BSA in PBS-T and then added to the plates. Plates were incubated at room temperature for two hours and then washed three times with PBS-T. A dilution series of antibodies was made in 1% BSA in PBS-T and then added to the plates. Plates were incubated at room temperature for one hour and then washed three times with PBS-T. The secondary antibody, goat anti-human IgG conjugated to peroxidase, was added at 1:10,000 dilution in 1% BSA in PBS-T to the plates, which were incubated for one hour at room temperature. Plates were washed three times and then developed by adding TMB substrate to each well. The plates were incubated at room temperature for five minutes, and then IN sulfuric acid was added to stop the reaction. Plates were read at 450 nm. ELISAs were repeated two more times.

### ELISA for Guinea Pig Serum

ELISA for serum was similar to as described above but with some modifications. Antigens with C-terminal Avi tags were site specifically biotinylated as described in above biotinylation section. Streptavidin was plated at 2 μg/mL overnight at 4° C. The next day, plates were washing three times with PBS supplemented with 0.05% Tween20 (PBS-T) and blocked with 1% BSA in PBS-T. Plates were incubated at room temperature for one hour and then washed three times with PBS-T. Biotinylated antigens were diluted to 2 μg/mL in 1% BSA in PBS-T and then added to the plates. Plates were incubated at room temperature for two hours and then washed three times with PBS-T. Serum was diluted starting at 1:50 (highest concentration) in 1% BSA in PBS-T, and then 5-fold down for a total of seven dilutions. Positive control antibodies for each antigen were included alongside. Plates were incubated at room temperature for one hour and then washed three times with PBS-T. The secondary antibody, goat anti-guinea pig IgG conjugated to peroxidase (highly cross-adsorbed), was added at 1:10,000 dilution in 1% BSA in PBS-T to the plates, which were incubated for one hour at room temperature. Plates were washed three times and then developed by adding TMB substrate to each well. The plates were incubated at room temperature for five minutes, and then IN sulfuric acid was added to stop the reaction. Plates were read at 450 nm. ELISAs were repeated two more times.

### ELISA for Humanized Monoclonal Guinea Pig Antibodies

ELISA for humanized monoclonal guinea pig antibodies was identical to ELISA for serum, with the exceptions that antibodies were diluted and screened (instead of serum), and the secondary was goat anti-human IgG conjugated to peroxidase (as used in ELISA for immunogens).

### Antibody-Dependent Cellular Phagocytosis Assay (ADCP)

The THP-1 phagocytosis assay was performed using 1 μM neutravidin beads (Molecular Probes Inc, Eugene, OR) coated with BG505 or 286 trimer. Sera were titrated and tested at a 1 in 100 dilution. Phagocytic scores were calculated as the geometric mean fluorescent intensity (MFI) of the beads multiplied by the percentage bead uptake as measured on a FACSAria II (BD biosciences, Franklin Lakes, New Jersey). THP-1 cells were obtained from the NIH AIDS Reagent Program and cultured at 37° C., 5% CO₂ in RPMI containing 10% heat-inactivated fetal bovine serum (Gibco, Gaithersburg, MD) with 1% Penicillin Streptomycin (Gibco, Gaithersburg, MD) and not allowed to exceed 4×10⁵ cells/ml.

### TZM-Bl Neutralization Assays

Serum and monoclonal antibody neutralization was assessed using the TZM-bl assay. This standardized assay measures antibody-mediated inhibition of infection of TZM-bl cells by molecularly cloned Env-pseudoviruses. Viruses that represent circulating strains (tier 2) were included. Murine leukemia virus (MLV) was included as an HIV-specificity control. Neutralization was measured as a reduction in luciferase gene expression after a single round of infection of TZM-bl cells in a 96 well plate. Results are presented as serum dilution required to inhibit 50% of virus infection (ID₅₀), or as antibody concentration required to inhibit 50% of virus infection (IC₅₀). Responses that were not at least 20 above background (ID₅₀ of 20) were considered negative and are shown as blue (ID₅₀<40); all other colors denote a positive response. Although some guinea pigs displayed low levels of neutralization towards MLV, these same guinea pigs had ID₅₀ responses of less than or equal to 20 for several HIV-1 pseudoviruses.

### REFERENCES

Ackerman, M. E., et al., A robust, high-throughput assay to determine the phagocytic activity of clinical antibody samples. J Immunol Methods, 2011. 366 (1-2): p. 8-19.
Bricault, C. A., et al., A multivalent clade C HIV-1 Env trimer cocktail elicits a higher magnitude of neutralizing antibodies than any individual component. J Virol, 2015. 89 (5): p. 2507-19.
Bricault, C. A., et al., HIV-1 Neutralizing Antibody Signatures and Application to Epitope-Targeted Vaccine Design. Cell Host Microbe, 2019. 25 (1): p. 59-72 c8.
Bricault, C. A., et al., Neutralizing Antibody Responses following Long-Term Vaccination with HIV-1 Env gp140 in Guinea Pigs. J Virol, 2018. 92 (13).
Cale, E. M., et al., Virus-like Particles Identify an HIV VIV2 Apex-Binding Neutralizing Antibody that Lacks a Protruding Loop. Immunity, 2017. 46 (5): p. 777-791 e10.
Cheng, C., et al., Consistent elicitation of cross-clade HIV-neutralizing responses achieved in guinea pigs after fusion peptide priming by repetitive envelope trimer boosting. PLOS One, 2019. 14 (4): p. c0215163.
Chuang, G. Y., et al., Structure-Based Design of a Soluble Prefusion-Closed HIV-1 Env Trimer with Reduced CD4 Affinity and Improved Immunogenicity. J Virol, 2017. 91 (10).
deCamp, A., et al., Global panel of HIV-1 Env reference strains for standardized assessments of vaccine-elicited neutralizing antibodies. J Virol, 2014. 88 (5): p. 2489-507.
Doria-Rose, N. A., et al., Developmental pathway for potent VIV2-directed HIV-neutralizing antibodies. Nature, 2014. 509 (7498): p. 55-62.
Gao, F., et al., Cooperation of B cell lineages in induction of HIV-1-broadly neutralizing antibodies. Cell, 2014. 158 (3): p. 481-91.
Georgiev, I. S., et al., Single-Chain Soluble BG505.SOSIP gp140 Trimers as Structural and Antigenic Mimics of Mature Closed HIV-1 Env. J Virol, 2015. 89 (10): p. 5318-29.
Griffith, S. A. and L. E. McCoy, To bnAb or Not to bnAb: Defining Broadly Neutralising Antibodies Against HIV-1. Front Immunol, 2021. 12: p. 708227.
Guo, Y., et al., Immunoglobulin genomics in the guinea pig (Cavia porcellus). PLOS One, 2012. 7 (6): p. e39298.
Haynes, B. F., et al., Strategies for HIV-1 vaccines that induce broadly neutralizing antibodies. Nat Rev Immunol, 2023. 23 (3): p. 142-158.
Hessell, A. J., et al., Achieving Potent Autologous Neutralizing Antibody Responses against Tier 2 HIV-1 Viruses by Strategic Selection of Envelope Immunogens. J Immunol, 2016. 196 (7): p. 3064-78.
Klasse, P. J., et al., Sequential and Simultaneous Immunization of Rabbits with HIV-1 Envelope Glycoprotein SOSIP.664 Trimers from Clades A, B and C. PLOS Pathog, 2016. 12 (9): p. e1005864.
Kong, L., et al., Supersite of immune vulnerability on the glycosylated face of HIV-1 envelope glycoprotein gp120. Nat Struct Mol Biol, 2013. 20 (7): p. 796-803.
Korber, B., et al., Polyvalent vaccine approaches to combat HIV-1 diversity. Immunol Rev, 2017. 275 (1): p. 230-244.
Kwong, P. D., et al., HIV-1 evades antibody-mediated neutralization through conformational masking of receptor-binding sites. Nature, 2002. 420 (6916): p. 678-82.
Lei, L., et al., Antigen-Specific Single B Cell Sorting and Monoclonal Antibody Cloning in Guinea Pigs. Front Microbiol, 2019. 10: p. 672.
Munro, J. B., et al., Conformational dynamics of single HIV-1 envelope trimers on the surface of native virions. Science, 2014. 346 (6210): p. 759-63.
Murji, A. A., et al., Elicitation of Neutralizing Antibody Responses to HIV-1 Immunization with Nanoparticle Vaccine Platforms. Viruses, 2021. 13 (7).
Nkolola, J. P., et al., Breadth of neutralizing antibodies elicited by stable, homogeneous clade A and clade C HIV-1 gp140 envelope trimers in guinea pigs. J Virol, 2010. 84 (7): p. 3270-9.
Pilewski, K. A., et al., Functional HIV-1/HCV cross-reactive antibodies isolated from a chronically co-infected donor. Cell Rep, 2023. 42 (2): p. 112044.
Sanders, R. W., et al., A next-generation cleaved, soluble HIV-1 Env trimer, BG505 SOSIP.664 gp140, expresses multiple epitopes for broadly neutralizing but not non-neutralizing antibodies. PLOS Pathog, 2013. 9 (9): p. e1003618.
Sarzotti-Kelsoe, M., et al., Optimization and validation of the TZM-bl assay for standardized assessments of neutralizing antibodies against HIV-1. J Immunol Methods, 2014. 409: p. 131-46.
Setliff, I., et al., High-Throughput Mapping of B Cell Receptor Sequences to Antigen Specificity. Cell, 2019. 179 (7): p. 1636-1646 e15.
Sok, D., et al., Recombinant HIV envelope trimer selects for quaternary-dependent antibodies targeting the trimer apex. Proc Natl Acad Sci USA, 2014. 111 (49): p. 17624-9.
Tong, T., et al., Impact of stabilizing mutations on the antigenic profile and glycosylation of membrane-expressed HIV-1 envelope glycoprotein. PLOS Pathog, 2023. 19 (8): p. e1011452.
Torrents de la Pena, A., et al., Immunogenicity in Rabbits of HIV-1 SOSIP Trimers from Clades A, B, and C, Given Individually, Sequentially, or in Combination. J Virol, 2018. 92 (8).
Wagh, K., et al., Completeness of HIV-1 Envelope Glycan Shield at Transmission Determines Neutralization Breadth. Cell Rep, 2018. 25 (4): p. 893-908 e7.
Walker, L. M., et al., Broad neutralization coverage of HIV by multiple highly potent antibodies. Nature, 2011. 477 (7365): p. 466-70.
Wang, Q., A. Finzi, and J. Sodroski, The Conformational States of the HIV-1 Envelope Glycoproteins. Trends Microbiol, 2020. 28 (8): p. 655-667.
Wu, X. Y., Zhi-Yong, Rational Design of Envelope Identifies Broadly Neutralizing Human Monoclonal Antibodies to HIV-1. Science, 2010. 329.
Xu, K., et al., Epitope-based vaccine design yields fusion peptide-directed antibodies that neutralize diverse strains of HIV-1. Nat Med, 2018. 24 (6): p. 857-867.
Zhu, P., et al., Electron tomography analysis of envelope glycoprotein trimers on HIV and simian immunodeficiency virus virions. Proc Natl Acad Sci USA, 2003. 100 (26): p. 15812-7.

SEQUENCES
Nucleotide sequence for oligo containing
15bp annealing antigen barcode
SEQ ID NO: 1:
5′CCTTGGCACCCGAGAATTCCANNNNNNNNNNNNNNNCCCATATAAGA*
A*A3′
Antigen barcode for CA/09
SEQ ID NO: 2:
GCAGCGTATAAGTCA
Antigen barcode for RSC3
SEQ ID NO: 3:
CAGTAAGTTCGGGAC
Antigen barcode for HT593.1
SEQ ID NO: 4:
TGACCTTCCTCTCCT
Antigen barcode for KNH1209.18
SEQ ID NO: 5:
GACCTCATTGTGAAT
Antigen barcode for 5768.04
SEQ ID NO: 6:
TAACTCAGGGCCTAT
Antigen barcode for 286.36
SEQ ID NO: 7:
CAGATGATCCACCAT
Antigen barcode for BG505 T332N
SEQ ID NO: 8:
ATCGTCGAGAGCTAG
Outer Reverse primer (RP) 1
SEQ ID NO: 9:
GGTAGGTGTGCACTCCACTGGTC
Outer RP 2
SEQ ID NO: 10:
CACCACTGTGGCCTTGTTKTCCTGG
Outer RP 3
SEQ ID NO: 11:
CACCACTGTGGCCTTGTTTTCGTTG
Outer RP 4
SEQ ID NO: 12:
GTGTTGTCCTTGCTGTCCTGATC
Inner RP 1
SEQ ID NO: 13:
GCTCAGGGAAGTAGCCCTTGAC
Inner RP 2
SEQ ID NO: 14:
CTCCTCACTCGAGGGYGGGAAYAGGCTG
Inner RP 3
SEQ ID NO: 15:
GTTCAGAGCCATCCACCTTCCAC
Forward Primer (FP) 1
SEQ ID NO: 16:
AATGATACGGCGACCACCGAGATCTACACTCTTTCCCTACACGACGCTC
Forward Primer (FP) 2
SEQ ID NO: 17:
AATGATACGGCGACCACCGAGATCT
Amino acid sequence for CDRH3- VH3-197-54
SEQ ID NO: 18:
AKAPYGYEGDV
Amino acid sequence for CDRH3- VH3-183-54
SEQ ID NO: 19:
ARYSYYRYSDGTYYSSGFDV
Amino acid sequence for CDRH3- VH3-80-54
SEQ ID NO: 20:
ARVPFYGYHYGAPDV
Amino acid sequence for CDRH3- VH3-167-75
SEQ ID NO: 21:
TLTYYWSADFDV
Amino acid sequence for CDRH3- VH1-58-54
SEQ ID NO: 22:
ARGYYDDSFNFQY
Amino acid sequence for CDRH3- VH3-114-75
SEQ ID NO: 23:
ARDYSSGFFPRSGFQH
Amino acid sequence for CDRH3- VH1-171-54
SEQ ID NO: 24:
ARCGAYYDFDV
Amino acid sequence for CDRH3- VH3-282-54
SEQ ID NO: 25:
AAEYYSRDNYYEII
Amino acid sequence for CDRH3- VH3-167-75
SEQ ID NO: 26:
ATGLAV
Amino acid sequence for CDRH3- VH3-167-75
SEQ ID NO: 27:
ATGLAV
Amino acid sequence for CDRH3- VH1-171-54
SEQ ID NO: 28:
ARDYGNDWYFDV
Amino acid sequence for CDRH3- VH1-151-54
SEQ ID NO: 29:
ATLTTTGEY
Amino acid sequence for CDRH3- VH1-151-54
SEQ ID NO: 30:
TTLTTTGEY
Amino acid sequence for CDRH3- VH1-151-54
SEQ ID NO: 31:
TTLTTTGEY
Amino acid sequence for CDRH3- VH1-151-54
SEQ ID NO: 32:
TTLTTTGEY
Amino acid sequence for CDRH3- VH3-197-54
SEQ ID NO: 33:
AREEGVQVYFDV
Amino acid sequence for CDRH3- VH3-167-75
SEQ ID NO: 34:
TSGISQ
Amino acid sequence for CDRH3- VH3-183-54
SEQ ID NO: 35:
VREEGGQVYFDV
Amino acid sequence for CDRH3- VH3-183-54
SEQ ID NO: 36:
VREEGGQVYFDV
Amino acid sequence for CDRH3- VH3-183-54
SEQ ID NO: 37:
VREEGGQVYFDL
Amino acid sequence for CDRH3- VH3-183-54
SEQ ID NO: 38:
VREEGGQVYFDV
Amino acid sequence for CDRH3- VH1-91-54
SEQ ID NO: 39:
ARGMGYFDV
Amino acid sequence for CDRH3- VH1-91-54
SEQ ID NO: 40:
ARGMGYFDV
Amino acid sequence for CDRH3- VH1-91-54
SEQ ID NO: 41:
VRGMGYFDV
Amino acid sequence for CDRH3- VH1-91-54
SEQ ID NO: 42:
ARGGGYFDV
Amino acid sequence for CDRH3- VH1-91-54
SEQ ID NO: 43:
ARGMGYFDV
Amino acid sequence for CDRH3- VH1-58-54
SEQ ID NO: 44:
ARTYYDDAFTFQY
Amino acid sequence for CDRH3- VH1-58-54
SEQ ID NO: 45:
ARTYYDDAFTFQY
Amino acid sequence for CDRH3- VH1-171-54
SEQ ID NO: 46:
ARCAGYYDFDF
Amino acid sequence for CDRH3- VH1-91-54
SEQ ID NO: 47:
VLRGTRMVTIDPHFDV
>112_22_HC Nucleotide sequence CDRH
SEQ ID NO: 48:
CAGGTCCAGCTCCAGGAATCAGGTCCCGGCCTTGTTAAACCTTCACAGTC
TTTAAGCATCACTTGTATGGTGAGTGGCTTTTCCATCACCACTACCAACT
ACTGGTGGCACTGGATTCGACAGCCCCCCGGCAAGAGCCTGGAGTGGCTG
GGAGAAATTAGATACGACGATGCTACTAAGTACAATCCCTCTCTGAAGTC
CAGATTAAGTATTACTCGCGACACCGGTAAGAATCAGTTCAGCCTGCAGT
TAACCTCAGTGACCACAGAGGATACTGCTACGTATTACTGTGCCAGAACC
TATTACGACGACGCCTTCACCTTCCAGTACTGGGGGCAAGGGACGCTCGT
CACAGTGAGCAGT
>112_23_HC Nucleotide sequence CDRH
SEQ ID NO: 49:
CAGGTGCAGCTCCAAGAAAGCGGACCGGGCCTGGTTAAACCAAGTCAGAG
CTTATCTATAACTTGTATGGTCAGTGGATTCAGTATTACCACCACAAATT
ATTGGTGGCACTGGATTCGGCAACCGCCCGGCAAGCGCCTGGAGTGGCTG
GGAGAAATGAAGTACGACGCTCATACCAAATACAACCCATCCGTGGAGAG
TCGCCTGAGCATCACTAGGGATACAGGAAAAAATCAATTTTTTCTGCAGC
TCACTTCCGTTACCACCGAAGATACCGCGACTTACTACTGCGCCAGAACA
TATTATGATGATGCCTTCACATTCCAATACTGGGGCCAGGGGACACTGGT
CACGGTTAGCTCC
Amino acid sequence for CDRL3- VK1-178
SEQ ID NO: 50:
QQGNSFPLT
Amino acid sequence for CDRL3- VL8-87
SEQ ID NO: 51:
GANDGSGSSFVYV
Amino acid sequence for CDRL3- VK1-274
SEQ ID NO: 52:
LQYGTSPYT
Amino acid sequence for CDRL3- VK2-3
SEQ ID NO: 53:
FQNTHSRNT
Amino acid sequence for CDRL3- VK1-140
SEQ ID NO: 54:
QQYDTYPFT
Amino acid sequence for CDRL3- VK3-280
SEQ ID NO: 55:
FQYYSGFT
Amino acid sequence for CDRL3- VK2-16
SEQ ID NO: 56:
VQTTHDPYT
Amino acid sequence for CDRL3- VK4-81
SEQ ID NO: 57:
MQTYSAPWT
Amino acid sequence for CDRL3- VK2-16
SEQ ID NO: 58:
VQTTHDPNT
Amino acid sequence for CDRL3- VK2-16
SEQ ID NO: 59:
VQTTHDPNT
Amino acid sequence for CDRL3- VK1-115
SEQ ID NO: 60:
QHNYGIS
Amino acid sequence for CDRL3- VL5-74
SEQ ID NO: 61:
GFWHSNRYV
Amino acid sequence for CDRL3- VL5-74
SEQ ID NO: 62:
GFWHSNRYV
Amino acid sequence for CDRL3- VL5-74
SEQ ID NO: 63:
GFWHSNRYV
Amino acid sequence for CDRL3- VL5-74
SEQ ID NO: 64:
GFWHSNRYV
Amino acid sequence for CDRL3- VK2-16
SEQ ID NO: 65:
VQSTYDPPT
Amino acid sequence for CDRL3- VK2-16
SEQ ID NO: 66:
VQTTHDPIT
Amino acid sequence for CDRL3- VK2-16
SEQ ID NO: 67:
VQSTQDPPT
Amino acid sequence for CDRL3- VK2-16
SEQ ID NO: 68:
VQSTQDPPT
Amino acid sequence for CDRL3- VK2-16
SEQ ID NO: 69:
VQSTQDPPT
Amino acid sequence for CDRL3- VK2-16
SEQ ID NO: 70:
VQSTQDPPT
Amino acid sequence for CDRL3- VK4-95
SEQ ID NO: 71:
QQFISGPPT
Amino acid sequence for CDRL3- VK4-95
SEQ ID NO: 72:
QQFIRAPPT
Amino acid sequence for CDRL3- VK4-95
SEQ ID NO: 73:
QQFISAPP
Amino acid sequence for CDRL3- VK4-95
SEQ ID NO: 74:
QQFISGPPT
Amino acid sequence for CDRL3- VK4-95
SEQ ID NO: 75:
QQFIRGPPT
Amino acid sequence for CDRL3- VK1-140
SEQ ID NO: 76:
QQYDSYPPLT
Amino acid sequence for CDRL3- VK1-140
SEQ ID NO: 77:
QQYDGYSPLT
Amino acid sequence for CDRL3- VK2-16
SEQ ID NO: 78:
VQTTHDPYT
Amino acid sequence for CDRL3- VK1-274
SEQ ID NO: 79:
LQTYISPRT
>109_1_HC Nucleotide sequence CDRH
SEQ ID NO: 80:
GAGGCCCAACTGGTAGAGAGCGGAGGAGGGCTGGTGCAACCTGGCGGGTC
TCTTAGACTGTCCTGCGTGGCTTCAGGCTTCACTTTCAACAGGTATGCTA
TGAACTGGATCCGGCAGGCCCCCGGGAAAGGATTGGAGTGGATTAGCATG
ATTTGGTACGACGGCTCCAAAATGTATTACGCGGATAGTGTTAAAGGCAG
ATTTACGATTTCCCGGGATAACGATAAAAATACACTCTATTTACAGATGA
GCTCACTGCGCCCAGAGGACACCGCCCTGTATTACTGTGCCAAGGCTCCC
TACGGATACGAGGGTGACGTTTGGGGAGCCGGCACATTGGTGACCGTGTC
CAGT
>109_1_LC Nucleotide sequence CDRL
SEQ ID NO: 81:
TGTGGAATACAGCTCACTCAACCTCCTAGCGCCAGCGCTAGCGTCGGGGA
CACTGTCAAGATTTCCTGCCAGGCATCACAATCAGTTTCAAGCTATCTTA
ACTGGTACCAACAGAAACCTGGCCAGGCGCCTAAACGCTTGATTTATGGC
GCTACCAACCTGGAAAGTGGCGTCCCTTCCAGGTTTTCAGGTTCTGGAAG
CGGTACTGGTTTTACCCTCACTATCAGCTCCCTGCAACCCGAAGATTTTG
CTACATATTACTGCCAGCAAGGCAATTCTTTCCCCTTGACCTTTGGCGGC
GGCACCAGACTGGAGATCAAG
>109_2_HC Nucleotide sequence CDRH
SEQ ID NO: 82:
GAAGAGCAGCTCGTTGAGTCAGGCGGAGGACTCGTACAGTCCGGAGGCTC
TCTCAAGCTGTCCTGCATGGCTTCCGGCTTTACGTTTTCCAGCTACCCTA
TGAGCTGGATTCGGCAGGCACCTGGCAAAGGCCTCGAGTGGATCTCAACC
ATCGGATCTCGCGGGTCAAAAACTTACTACGCCGGCTCCGTGAAGGGCCG
TTTCACAATCAGCAGGGACAATGGCAAGAATACGTTGTACCTCCAGATGA
GTAGTTTGCGCCCTGAAGACACGGCAGTTTATTACTGCGCCCGGTACTCC
TATTACCGCTATAGTGATGGAACATATTACTCCAGCGGCTTCGACGTTTG
GGGAGCTGGGACCTTGGTGACCGTCTCCTCC
>109_2_LC Nucleotide sequence CDRL
SEQ ID NO: 83:
CAGCCTGTGCTGACCCAACTGCCTTCAGCAAGCGCCTCAGTGGGGCAGAC
ATTAAAGCTGACTTGTACCCTGAGCAGCGGGTATAGCAACTATAATGTGA
ATTGGTACCAACAATCACAGGGGAAGTCTCCCCGCTTTGTGATGCGCGTC
GGAACTAGCGGTATAGTGGGGAGCAAGGGAGACGGCATACCAGATAGGTT
TTCAGGTAGTGGTAGCGGCTGGGATCGATATTTGACTATCCAGAACATCC
AGGAGGAGGATGAGAACGTTTACTATTGTGGCGCTAACGATGGCTCTGGC
TCTTCCTTTGTCTACGTCTTCGGGGGAGGAACCAAACTCACGGTCCTT
>109_3_HC Nucleotide sequence CDRH
SEQ ID NO: 84:
GAGGAACAGCTGGTAGAAAGTGGAGGGGGACTGGTTCAACCGGGCGGATC
ACTGAAGCTTTCTTGCGTGGCGAGTGGATTCACATTTAGCGACTATTGGA
TGAGCTGGATCCGGCAGGCTCCTGGTCGCGGTTTGGAGTGGCTGACCTAC
ATAAATTATGACGGCGATGATACGTACTACACAGACAGCGTGAAGGGGCG
GTTTACCATCTCCCGAGACAACTCAAGGAATGTCCTGTTCTTGCAGATGT
CACGCCTCAAGCCCGAAGATACCGCTGTGTATTACTGCGCTCGGGTGCCC
TTTTATGGATACCATTATGGGGCCCCAGACGTGTGGGGTGCGGGCACCCT
CGTGACAGTGTCCTCT
>109_3_LC Nucleotide sequence CDRL
SEQ ID NO: 85:
TGTGACATTCAGCTCACACAGCCGGCCTCCGCGAGCGCCAGCGTGGGCGA
CACTGTGAAGATTAGCTGTCAGGCCAGCCAGAATATCTATAGCTATCTCT
CTTGGTACCAGCAAAAGCCCGGACAGGCCCCGAAGCTCCTTATCTACTAT
GCGAACCGCCTCGCAAGCGGCGTCCCGTCACGCTTCAGCGGAGCAGGGAG
TGGAACTGACTTTACACTCACAATCTCTTCCCTGCAGCCTGAAGATTTTG
CCACATACTATTGTTTGCAATACGGCACATCACCTTACACCTTCGGCGCC
GGCACCAAACTGGAAATAAAG
>109_4_HC Nucleotide sequence CDRH
SEQ ID NO: 86:
GAGGTGCAGCTGGTGGAGTCCGGCGGGGGGCTGGTGAAGCCAGGCGAGTC
ACTTCGCCTTTCTTGCGTGGCTAGCGGTTTTACCTTTAGCTCATACTGGA
TGAACTGGATCCGTCAGGCCCCTGGTAAAGGGCTGGAGTGGATCAGCGCC
CTGAATAGCGATGGCAGCACCACTCTTTACGCGGACAGTGTCAAGGGCCG
GTTTACCATTAGCCGAGATAACGGTAAGAACACCCTCTACCTGCAAATGA
GCTCACTGAGACCTGAGGACACAGCAATCTACTATTGCACGCTGACTTAT
TACTGGAGTGCAGACTTCGACGTCTGGGGCGCAGGTACCCTCGTGACAGT
TTCAAGC
>109_4_LC Nucleotide sequence CDRL
SEQ ID NO: 87:
GACGTGGTAATGACACAAACCCCCCTGTCACTGTCCGTCAGTCCAGGTGA
ACCAGCGTCCATTTCCTGTAGAGCTAGCCAGTCACTGCTTCACTCCAATG
GTAAGACATATCTTCATTGGGTGGTTCATAAACCAGGGCAAGCTCCACGC
GGCATGATCTACCAGGTATCAAATAAGTATAGTGGAACCCCAGAGCGCTT
CAGTGGAAGTGGGTCCGGGACTGATTTCACTCTCAAGATAAGTCGCGTGG
AAGCAGAAGACGCCGGAGTGTACTATTGTTTCCAGAACACTCATTCCAGA
AATACTTTTGGGGGCGGCACAAAATTAGAGATTAAG
>109_5_HC Nucleotide sequence CDRH
SEQ ID NO: 88:
CAAGTGCAGCTGCAGGAATCAGGACCCGGCCTTGTCAAGCCCTCTCAGAG
CCTCAGCATCACCTGCATGGTGTCTGGATTTAGCATCACAACCACAAACT
ATTGGTGGCACTGGATCAGGCAGCCACCTGGCAAATCCCTGGAATGGCTG
GGGGAAATCAGCTACGACGGCAATACCAAAAGCAACCCATCAGTCAAGTC
AAGACTGTCTATCACTAGGGACACCGGCAAAAATCAGTTCTCCCTCCAGC
TGACCAGCGTGACAACGGAAGACACGGCTACATACTATTGTGCGCGCGGA
TATTACGACGACAGTTTTAATTTCCAGTACTGGGGACAGGGCACCCTGGT
TACTGTGTCTTCC
>109_5_LC Nucleotide sequence CDRL
SEQ ID NO: 89:
GATATCCAGATGACTCAAAGCCCCAGTTTCCTTTCAGCATCTCTTGGGGA
CCGAGTAACCATCACTTGTAAGGCATCTCAAAACATTCATCGGAATCTGG
CCTGGTACCACCAGAAACCTGGAAAAGCCCCCCGGCTGTTGATTTATAGT
ACAAGCAACCTGGACTCAGGTGTGCCTTCTCGCTTCTCCGGGAGCGGCAG
TTGGACTGACTACGCATTTACTATTTCAGCTGTCGAGCCCGACGATGTAG
GAATTTATCATTGTCAACAGTATGATACTTACCCTTTTACATTCGGAGGG
GGCACTAAGCTCGAAATCAAG
>112_1_HC Nucleotide sequence CDRH
SEQ ID NO: 90:
GAGGTCCAGCTGGTGGAAAGTGGTGGCGGCTTGGTGCAACCAGGGGGCTC
TCTGAGACTGAGCTGTGTCGCTTCCGGATTTTCCTTCGACAATTATGGTA
TGACCTGGATTAGGCAAGCCCCTGGCAAAGGCTTGGAATGGATTAGCGCC
ATCAGCTTCTCTGGGGGTAACACTAATTATGCAGACAGTGTGAAGGGGAG
GTTCACAATTTCCCGCGATAACGGAAAGAACGCTCTCTACCTCCAGCTCT
CCTCACTCCGACCTGAGGACACCGCCACTTATTATTGCGCTCGAGACTAT
TCAAGCGGCTTCTTCCCACGCTCCGGGTTCCAACACTGGGGACAGGGAAC
GTTGGTGACCGTGTCCTCT
>112_1_LC Nucleotide sequence CDRL
SEQ ID NO: 91:
CAAACACTGTTGACACAAACTCCAGATAGTTTAGCGGTGTCTCCCGGCGA
GACGGTTACCCTGAATTGTCGGGCATCCCAGGGGGTCTCAGACTACCTCG
AGTGGTATCAACAGAAGCCCGGACGTGCCCCTCGCCTGCTCATATACTCA
GCCAGTGACAGAGCTACCGGGGTGCCTGCCCGCTTTAGCGGAAGCGGATC
TGGGACAGATTTTACTTTAACAATTTCCAGTCTGCAACCACAGGACATTG
CGGTCTATTATTGTTTTCAGTACTATTCAGGATTTACCTTTGGTGGTGGG
ACTAAATTAGAAATTAAA
>112_2_HC Nucleotide sequence CDRH
SEQ ID NO: 92:
CAGGTCCAGCTTCAGGAGAGTGGATCCGGCTTAGTGGGCCCATCCCAGAC
TCTTTCCCTCGCATGTTCTGTGAGCGGGTTCAGCATTACCACAAGCGGAT
ACTCTTGGGCTTGGATTCGGCAACCTCCCGGCAAGTCACTGGAGTGGATG
GGCCACATTTGGTACGATGCCGACACTTACTACAACCCCTCCATCAAGAA
CAGGATTAGCATTTCCCGTGACACCGGGAAAAATCAGTTTTCCCTCCAGC
TCAGCTCCATCACCACGGAGGATACAGCAATCTATTACTGTGCTCGATGT
GGGGCCTACTATGATTTTGACGTCTGGGGGGCCGGCACTTTGGTGACTGT
AAGCTCA
>112_2_LC Nucleotide sequence CDRL
SEQ ID NO: 93:
GACGTCGTAATGACACAGACTCCATTGCTGCTGTCCGTCTCTCCCGGAGA
GCCTGCCAGCATGTCCTGCAAGAGCAGCCAAAGCCTGTGGAACTCAGATG
GTTACACTTACTTACACTGGCTGGTGCATAAACCAGGCCAGGCCCCACGG
GGCGTGATCTATGAAGTGTCTGATAAATATTCATGGACCAGCGAACGGTT
TACAGGCGGGGGATCCGGAACAGACTTTACTCTGAAGATATCTCGCGTAG
AGGCCGAGGATGCCGGGGTGTACTACTGCGTGCAGACAACCCATGACCCA
TATACTTTTGGAGCGGGGACTAAACTCGAGATTCGG
>112_3_HC Nucleotide sequence CDRH
SEQ ID NO: 94:
GAGGTCCAGCTGGTTGAGTCCGGAGGTGGGCTCGCTCAGCCGGGTGGTTC
CTTGCGACTCAGCTGCTTGGCGAGCGGTTTTACTTTCAACGAGTCTTGGA
TGTCTTGGGTGCGGCAGGTGCCCGGCAAGGCCCTTGAGTGGCTGGCAAAC
ATTAACGGAGATAGTACCAAAATGAACTACGCTGATAGCGTCAAAGGTAG
ATTCACCATGTCCAGGGACTCTGGCAAAAACACCCTGTACCTTCAAATGA
ACGGCTTGCGAAGCGCCGACACCGCCTTGTACTACTGTGCCGCCGAATAT
TACTCACGCGACAACTATTATGAAATAATTTGGGGCGCCGGAACTCTGGT
GACCGTATCTTCC
>112_3_LC Nucleotide sequence CDRL
SEQ ID NO: 95:
GACATCGTGATGACTCAGTCCCCTGCTTCACTGGCAGTGACACCTGGTGA
GTCCGCCACCATACACTGTAAGAGCTCTCAATCCCTGCTGTATTCATATG
ACAACAAAAACTACCTCATATGGTATCAGCAAAAGCCAGGCCAGTCTCCA
AAATTGTTGATTTCTTTAGCCTCTACCCGTAACACAGGAGTCTCAGATCA
CTTTAGCGGGTCTGGTTCTGGGACCGACTTCACACTCACAATCTCAAGGG
TGCAGGCTGAGGATGTTGCGGATTATTACTGCATGCAAACGTACTCCGCA
CCATGGACATTCGGCGCTGGAACAAAGTTAGAGATTAAG
>112_4_HC Nucleotide sequence CDRH
SEQ ID NO: 96:
GAGGTACAACTGGTTGAGTCAGGGGGGGGATTGGTGAAACCAGGCGAAAG
CCTTCGCTTGTCTTGTGTGGCTAGTGGATTCACGTTCTCCTCATACTGGA
TGAACTGGATTCGCCAGGCTCCAGGGAAGGGGCTCGAGTGGATCTCTACC
ATCAATTCCGACGGCACTAATACTTATTATTCCGACAGTGTGAAAGGCAG
ATTCACCATTAGTCGCGATAATGGGAAGAATACTCTGTACCTCCAGATGA
GCAGCTTGCGTCCTGAAGATACTGCCATGTTCTATTGCGCTACTGGCCTG
GCCGTGTGGGGCGCCGGGACTCTGGTGACTGTTTCATCA
>112_4_LC Nucleotide sequence CDRL
SEQ ID NO: 97:
GATGTGGTCATGACACAGACCCCACTCCTTTTGAGTGTGTCTCCCGGCGA
GCCTGCGTCTATTTCATGTCGGAGTTCCCAGTCCCTCTTATATAGCGACG
GCTCCACCCTGCTGCACTGGTTAGTACATAAGCCAGGCCAAGCCCCAAGG
GGAGTGATCTACGAAGTTAGCGATAAGTACTCCTGGACCCCTGAAAGGTT
CACGGGCTCTGGCTCAGGAACCGATTTTACACTTAAGATCTCTCGGGTCG
AGGCTGAGGACGCTGGAGTCTACTACTGCGTTCAGACTACGCACGATCCC
AACACCTTCGGCGGAGGCACCAAGCTCGAAATAAAG
>112_7_HC Nucleotide sequence CDRH
SEQ ID NO: 98:
CAGGTCCAATTGCAGGAATCCGGCCCAGGACTTGTGAAGCCTTCACAGAC
ACTGAGCCTGACATGCACAGTGACAGGCTTTTCCATCACAACGAACACGT
ATTATTGGTCCTGGATCCGCCAACCTCCTGGTAAATCACCCGAATGGCTC
GGTATTATAGACAGCGGAGGTGACACGACCTACAGCCCCTCATTGAAGAG
CCGAATTAGTATCTCTAGGGACACTGGCAAAAGTCAGTTTTCTCTTCAGC
TGCGTTCCGTGACTCCTGAGGACACAGCCACTTATTACTGCGCTACTCTG
ACAACCACGGGAGAATATTGGGGTCAGGGTACCCTGGTAACTGTGTCATC
AG
>112_7_LC Nucleotide sequence CDRL
SEQ ID NO: 99:
CAGGAGTTAGTGACTCAAGAACCTAGTCTCTCCGCACCCCCCGGAACGGC
AGTCCGCTTAACTTGCACACTGCGGTCCGATCTTAGTTCCTACCGCATTT
TCTGGTACCAGCAGAAGCCCGGGTCTCCCCCCCGGTTCCTCCTTCATTAC
CATACCGATTCTGACAAGCAGCTCGGCTCTGGGTTCCCTTCTCGGTTTAG
TGGCAGTACCAGCGCCAATGCTGGTGTGTTACGGATTTCCGGTCTCCAGC
CCGAGGATGAGGCGGACTTCTATTGCGGCTTTTGGCACAGTAACCGATAT
GTGTTTGGTGGCGGCACTAAACTTACAGTGCTC
>112_8_HC Nucleotide sequence CDRH
SEQ ID NO: 100:
CAGGTGCAACTCCAGGAATCCGGACCCGGATTAGTGAAGCCTAGCCAGAC
CCTCAGCCTCACATGTACCGGGACTGGAGTCTCTATCACCGCCAGCACTT
ATTACTGGAGCTGGATTCGCCAACCCCCAGGTAAGAGTCCCGAGTGGATG
GGCATCATCGACTCAGACGGCGACACAGCATATTCTCCATCTCTCAAAAG
CCGAATATCTATCTCTAGAGATACTGGTAAGAACCAGTTCAGCCTTCAGC
TCAGGAGCGTCACTCCAGAGGATACAGCTACATACTTCTGTACAACACTG
ACAACCACTGGCGAGTATTGGGGCCAGGGAACTCTGGTGACCGTCAGCTC
A
>112_8_LC Nucleotide sequence CDRL
SEQ ID NO: 101:
CAGGAACTGGTTACCCAGGAGCCTTCCCTGTCTGCCCCCCCAGGCACTGC
CGCTCGCTTGACCTGCACATTAAGGAGTGACCTTTCTTCTTACAGGATCT
TCTGGTATCAGCAGAAACCAGGCTCTCCTCCGAGATTTCTGTTGCACTAT
CATACTGACTCTGATAAGCAGTTGGGCAGCGGCTTCCCAAGCCGGTTCTC
TGGATCCACTTCCGCGAATGCCGGGGTTTTAGGAATTAGTGGGCTGCAGC
CAGAGGATGAGGCCGATTACTATTGCGGATTTTGGCATAGCAACAGGTAT
GTGTTTGGTGGTGGAACAAAACTGACCGTACTT
>112_9_HC Nucleotide sequence CDRH
SEQ ID NO: 102:
CAGGTGCAGCTGCAAGAATCCGGGCCCGGACTTGTGAAGCCTTCTCAAAC
CCTCTCACTGACCTGTACAGGGACAGGAGTGTCTATCACCGCTAATACTT
ACTATTGGAGCTGGATTCGCCAGCCCCCTGGGAAATCTCCTGAGTGGATG
GGAATAATTGACAGCGACGGCGACACGGCATACAGCCCTTCTCTGAAATC
CCGGATATCCATCTCCAGAGACACTGGTAAGAATCAGTTCAGCCTGCAGT
TACGCTCCGTGACCCCTGAGGATACTGCAACCTATTTTTGCACCACGCTG
ACAACAACTGGCGAGTATTGGGGCCAGGGAACTCTCGTTACAGTGTCCTC
T
>112_9_LC Nucleotide sequence CDRL
SEQ ID NO: 103:
CAAGAGCTCGTTACGCAGGAGCCCAGCCTGTCCGCCCCCCCAGGCACCGC
CGCAAGGCTGACATGTACCCTGAGAAGTGATCTCAGCAGCTATAGGATCT
TCTGGTACCAACAGAAACCTGCCTCCCCCCCACGCTTTTTGCTCCACTAT
CATACCGATAGTGATAAGCAGCTCGGCTCCGGCTTCCCTTCTCGATTTTC
AGGAAGCACAAGCGCTAACGCCGGGGTTCTCGGTATCTCCGGTCTGCAGC
CTGAGGATGAAGCTGACTATTATTGTGGTTTCTGGCATAGTAACAGGTAC
GTCTTCGGGGGCGGTACAAAACTCACAGTGCTG
>112_10_HC Nucleotide sequence CDRH
SEQ ID NO: 104:
CAGGTCCAGCTGCAGGAAAGTGGTCCCGGACTGGTGAAACCTTCACAAAC
ACTCAGCCTGACCTGTACAGGAACTGGGGTTAGTATTACAGCAAGTACTT
ATTACTGGTCATGGATCAGGCAGCCACCCGGTAAGTCTCCTGAGTGGATG
GGTATTATTGACAGTGATGGGGACACGGCTTACAGCCCAAGCTTGAAGTC
AAGAATATCCATCAGCAGGGATACAGGAAAAAATCAATTCTCTCTTCAAC
TTCGTAGCGTGGCACCCGAGGATACCGCCACGTATTTCTGTACAACTCTT
ACTACAACTGGTGAGTACTGGGGCCAGGGGACTTTAGTTACCGTCTCCTC
T
>112_10_LC Nucleotide sequence CDRL
SEQ ID NO: 105:
CAGGAACTGGTAACCCAGGAGTCTTCTCTGAGCGCCCCACCCGGGACCGC
TGCACGACTGACTTGCACTCTCCGCAGCGACCTGAGTTCCTATCGCATCT
TCTGGTATCAGCAGAAACCAGGCTCTCCTCCTAGATTTCTGCTGCACTAC
CACACAGACAGTGATAAGCAGTTAGGAAGCGGCTTCCCTAGCCGGTTCTC
TGGGTCAACCTCAGCAAATGCCGGGGTTCTGGGCATCTCAGGCCTTCAGC
CTGAGGACGAGGCTGCATATTATTGTGGGTTTTGGCATTCTAATCGCTAC
GTGTTCGGCGGGGGCACCAAGCTGACAGTGCTC
>112_11_HC Nucleotide sequence CDRH
SEQ ID NO: 106:
GAAGAGCATCTCGTCGAGAGCGGCGGCGGCCTGGTTCAGCCTGGGGGGCC
CCTCCAGCTGAGCTGCGTCGCAAGTGGCTTCACTTTTTCTAACTATGCAA
TGAGTTGGATCAGACAAGCGCCTGGGAAAGGCCTGGAATGGATTTCAGCG
ATAAATAGCGTCTCTAAGATCTATTACGCGGATTCCGTCAAGGGCAGGTT
CACAATCAGCAGGGACAACGACAAAAACACGCTCTACCTCCAGATGAGCT
CCTTAAGGCCAGAGGACACCGCAGTGTACTACTGCGCTAGGGAAGAAGGC
GTTCAGGTTTACTTCGATGTCTGGGGAGCGGGCACGCTGGTCACTGTGTC
TTCT
>112_11_LC Nucleotide sequence CDRL
SEQ ID NO: 107:
GACGTCGTGATGACCCAGACTCCGCTGCTGCTGAGCGTATCTCCTGGTGA
GCCAGCCTCAATCAGCTGTCGAAGCTCCCAGTCCTTGCTCTATTCTGACG
GATATACTTATCTCCATTGGCTTGTTCACAAGCCTGGGCAGGCTCCACGC
GGAGTTATATACGAGGTGAGCGACAAATATTCCTGGACTCCCGAGCGATT
CACCGGGAGTGGCTCTGGGACTGACTTCACTCTGAAGATCAACCGGGTGG
AGGCTGAAGACGCTGGTCTGTATTACTGCGTTCAGAGTACCTATGACCCT
CCTACTTTTGGGGCCGGTACAAAACTGGAGATTAAG
>112_12_HC Nucleotide sequence CDRH
SEQ ID NO: 108:
GAGGTTCAGCTCGTCGAGTCAGGCGGAGGATTAGTGAAACCTGGGGAGTC
CCTCAGGCTCAGTTGTGTGGCTAGCGGCTTCGCCTTCTCCACCTACTGGA
TGAATTGGGTTAGACAGGCACCCGGGAAAGGGTTAGAATGGATTTCAGGC
ATCTCCGGAGACGGCTCAAGCACCTATTACGCCGACTCAGTGCAGGGGCA
GTTCACAATCTCCCGCGATAACGACAAGAACACTCTGTCCCTCCAGATGT
CTTCTTTAAGACCTGAAGACACAGCAATGTATTACTGCACTTCCGGCATC
AGTCAGTGGGGTGCCGGGACACTTGTGACCGTGTCATCT
>112_12_LC Nucleotide sequence CDRL
SEQ ID NO: 109:
GACGTCGTCATGACGCAGACGCCCCTGCTTTTGTCCGTGTCACCCGGTGA
GCCCGCTTCAATTTCCTGCAGGAGCTCCCAATCCCTTCTCGATTCTGACG
GATATAACTACCTGCACTGGGTCGTTCACAAGCCCGGGCAGGCCCCCCGC
GGAGTGATCTACGAGGTCTCTGACAAGTATAGCTGGACCCCAGAGCGGTT
TACTGGATCCGGCAGCCGAACAGACTTCACACTCAAAATTAGTAGGGTGG
AAGCCGAAGACGCCGGAGTCTACTACTGTGTACAGACAACTCACGACCCC
ATAACTTTTGGCGGGGGGACTAAGCTCGAAATCAAA
>112_13_HC Nucleotide sequence CDRH
SEQ ID NO: 110:
GAGGAGCAGCTGGTCGAATCCGGTGGGGGACTGGTGCAGCCAGGCGGCTC
CCTGAAACTGTCTTGCATGGCTTCCGGCTTTACATTTAGCTCCTACGCCA
TGACCTGGATAAGGCAGGCACCAGGCAAGGGTCTTGAGTGGATCAGTGCC
ATTAGATCCTCAGGGTCAAACACTTATTATGCTGGAAGCGTAAAAGGCCG
TTTTACCATAAGCAGGGATAATGATAAGAATACCTTGTACCTGCAGATGT
CTAGCCTCAGACCAGAGGACACAGCCGTCTACTATTGCGTTCGCGAGGAA
GGGGGACAGGTCTATTTTGACGTTTGGGGTGCGGGGACCCTCGTGACCGT
GTCATCT
>112_13_LC Nucleotide sequence CDRL
SEQ ID NO: 111:
GATGTGGTTATGACGCAGACGCCGCTGCTCCTGAGCGTGTCACCCGGCGA
ACCCGCCTCTATTAGTTGTAGGTCCTCCCAATCCCTGTTATATAGCGACG
GCTATACATACTTGCACTGGCTGGTGCACAAACCCGGTCAGGCCCCCCGT
GGAGTGATCTACGAGGTGTCCGACAAATATAGTTGGACTCCAGAGCGATT
CAGTGGCTCCGGCTCTGGTACGGATTTTACGTTAAAGATTTCTCGGGTGG
AGGCCGAGGACATCGGCGTGTACTACTGTGTGCAGTCTACTCAAGACCCC
CCCACTTTCGGAGCTGGGACTAAACTGGAAATCAAA
>112_14_HC Nucleotide sequence CDRH
SEQ ID NO: 112:
GAGGAACAATTAGTCGAGTCAGGAGGGGGACTTGTTCAGCCCGGCGGGTC
ACTGAAATTAAGCTGCATGGCCTCAGGTTTCAGCTTTAGCTCCTACGCCA
TGACATGGATACGTCAGGCACCAGGAAAGGGCCTGGAATGGATCAGCGCC
ATCAGGTCCTCCGGCAGTAACACTTACTACGCGGACTCTGTCAAAGGGCG
TTTTACAATCTCTCGGGATAATGACAAGAATATGCTCTATCTGCAGATGT
CCTCCCTGAGGCCGGAGGACACCGCAGTGTACTATTGTGTACGCGAAGAA
GGAGGCCAGGTGTACTTCGACGTGTGGGGTGCCGGTACTCTGGTCACCGT
CTCCTCC
>112_14_LC Nucleotide sequence CDRL
SEQ ID NO: 113:
GAGATCGTCATGACCCAGACGCCATTGCTGCTCTCAGTTTCTCCAGGGGA
GCCCGCAAGCATTAGCTGCCGTTCATCTCAGTCTCTGCTGTACAGTGACG
GTTACACTTACCTCCACTGGCTCGTTCATAAGCCTGGGCAGGCACCCCGC
GGAGTGATTTACGAGGTGTCAGACAAGTATTCCTGGACACCAGAACGATT
CACGGGGTCAGGATCCGGAACAGACTTCACATTGAAGATTTCCCGCGTGG
AAGCAGAAGACATTGGGGTGTATTATTGTGTCCAGTCAACCCAGGATCCC
CCTACGTTCGGGGCAGGCACCAAGCTCGAAATCAAG
>112_15_HC Nucleotide sequence CDRH
SEQ ID NO: 114:
GAGGAGCAGCTCGTCGAGAGTGGCGGGGGACTGGTGCAGCCAGGAGGATC
TCTTAAACTCTCCTGTATGGCCAGTGGATTTAGTTTTTCATCCTACGCCA
TGACCTGGATTAGGCAGGGTCCCGGGAAGGGCCTGGAATGGATCAGCGCT
ATTCGTTCTTCTGGCAGCAAGACATATTATACTGGAAGCGTGAAAGGTCG
CTTTACTATTTCCAGAGATAATGACAAGAACATGCTCTATTTACAGATGT
CCAGCCTGAGGCCAGAGGATACAGCAGTGTACTATTGTGTACGGGAGGAG
GGGGGGCAAGTGTACTTTGATCTCTGGGGGGCAGGTACCTTGGTCACCGT
GTCCTCTG
>112_15_LC Nucleotide sequence CDRL
SEQ ID NO: 115:
GAGATTGTTATGACTCAGACTCCCCTGCTGCTGAGCGTGTCACCGGGCGA
ACCTGCATCAATTAGTTGCCGCAGTTCTCAGAGCCTCCTCTATTCAGACG
GCTACACATACTTGCATTGGTTGGTTCACAAGCCCGGACAAGCACCAAGG
GGCGTTATCTATGAAGTCTCCGACAAATACTCCTGGACTCCTGAACGCTT
CACCGGCTCTGGTTCCGGTACAGATTTCACCCTTAAAATCTCCAGAGTAG
AGGCAGAGGACATAGGAGTTTACTACTGTGTGCAGTCAACTCAGGACCCC
CCGACATTCGGTGCAGGTACAAAACTCGAAATCAAG
>112_16_HC Nucleotide sequence CDRH
SEQ ID NO: 116:
GAGGAGCAGCTGGTGGAATCCGGTGGTGGATTGGTCCAGCCCGGTGGCAG
TCTGAAACTTAGCTGCATGGCCTCCGGCTTCTCCTTCTCCTCTTATGCAA
TGACTTGGATCCGGCAGGCACCTGGGAAAGGACTTGAATGGATAAGCGCT
ATTAGAAGCAGCGGCTCTAATACGTACTACGCCGACAGCGTTAAAGGGAG
ATTCACCATTAGTCGAGATAACGACAAAAACATGTTGTACTTACAGATGA
GCTCTTTAAGGCCTGAAGACACAGCCGTTTACTATTGTGTACGTGAGGAG
GGCGGACAAGTCTATTTTGACGTCTGGGGAGCCGGTACGTTGGTGACTGT
TAGCTCC
>112_16_LC Nucleotide sequence CDRL
SEQ ID NO: 117:
GAAATTGTAATGACTCAGACCCCCTTGCTGCTTAGCGTCAGCCCTGGTGA
GCCGGCATCCATTAGCTGTCGGAGTTCACAAAGTCTGCTGTACAGCGATG
GTTACACCTACCTCCACTGGCTTGTTCACAAGCCGGGACAGGCCCCACGG
GGGGTGATTTATGAAGTGTCAGATAAGTATTCTTGGACTCCTGAACGCTT
CACAGGATCCGGAAGCGGCACCGACTTTACTCTTAAGATCAGCAGAGTAG
AGGCAGAGGACATCGGCGTCTACTATTGTGTGCAGTCAACGCAAGATCCC
CCAACTTTCGGTGCCGGCACCAAGCTCGAAATCAAA
>112_17_HC Nucleotide sequence CDRH
SEQ ID NO: 118:
CAGATGCAGCTGCAAGAAAGCGGGCCAGGGCTCGTGAAGCCGAGCCAGAC
TCTGTTTTTGACCTGTAGCGTTAGCGGGTTTTCCATAACCACGTCCGGGA
GTGGCTGGAGTTGGATTCGGCAACCCCGCGGGAAGACTCTGGAGGTTTTA
GGGGGAGTTGCATACAACGGAGGGACTGGCTATAATCCATCCATAGAATC
CCGGATTTCCATATCTAGGGATACTGGCAAAAACCAATTTAGCCTGCAGG
TGAACTCAGTGACCGAAGAAGACACCGCCACATACTATTGCGCTAGAGGG
ATGGGATATTTCGACGTTTGGGGGGCGGGTACTCTGGTTACTGTGAGCTC
A
>112_17_LC Nucleotide sequence CDRL
SEQ ID NO: 119:
GAGACAGTGATGACACAGTCTCCCGCCACTATGGCCGTTTCACCAGGTGA
GAGAGTAACCATACATTGTAAATCCAGCCAGTCTCTGTTTTACAGCGGTA
ACAATAAGCATCTCCTTAGCTGGTATCAGCAGAAGCCTGGACAGAGCCCT
AAGCTGCTCATTTACTTCACGTCCACGAGAGCCAGTGGAGTGCCTGACCG
GTTTTCCGGATCCGGGTCTGGAACCGACTTCACTCTCACAATTAATTCCC
TGCAGGCCGAGGACGTAGCCGATTATTTTTGCCAGCAGTTCATTTCTGGG
CCACCTACATTCGGGGCTGGAACAAAATTGGAAATCAAG
>112_18_HC Nucleotide sequence CDRH
SEQ ID NO: 120:
CAGATGCATCTGCAGGAGAGCGGCCCCGGCCTCGTCAAACCATCACAGAC
GTTGTTCCTGACATGTTCAGTTAGCGGGTTTAGTATCACAACAAGTGCCT
ATGGCTGGAGCTGGATACGGCAACCCAGAGGCAAAACCCTGGAAGTAATG
GGTGGCATTGCCTATAATGGAGGAACAGGCTATAACCCAAGTATCAAATC
CCGGATTTCCATATCCAGGGACACAGGTAAGAACCAATTTTCACTTCAGC
TCAACAGCGTGACCGAGGAAGACACGGCAACCTACTATTGCGCTAGGGGA
ATGGGGTACTTTGATGTGTGGGGCGCCGGAACACTGGTTACCGTAAGCTC
C
>112_18_LC Nucleotide sequence CDRL
SEQ ID NO: 121:
GATATCGTTATGACTCAAAGTCCAGCAACCATGGCCGTGTCTCCCGGAGA
GAGAGTGACTATCCACTGCAAGTCCTCCCAGAGTCTCTTCTATTCAGGCA
ATTCCAAACACCTGTTATCATGGTATCAGCAGAGACCCGGTCAGTCCCCG
AAGCTGCTGATCAGTTGGGCATCCACCCGCGCGAGTGGAGTCCCTGATAG
ATTCAGCGGGTCTGGCTCTGGTACAGACTTCACACTTACGATCTCTTCAC
TCCAGGCTGAAGACGTAGCAGTGTACTTCTGTCAGCAGTTTATCCGAGCC
CCTCCCACATTTGGAGCCGGCACTAAACTCGAAATTAAG
>112_19_HC Nucleotide sequence CDRH
SEQ ID NO: 122:
CAGATGCAGTTACAGGAATCAGGTCCTGGCCTTGTGAAGCCATCCCAGAC
CCTTTTTCTCACCTGTTCCGTGTCAGGCTTCAGTATTACTACGAGTGGAT
ACGGATGGAGCTGGATTAGGCAGCCACGGGGTAAAACACTGGAGGTCATG
GGCGGAATCGCATATAACGGGGCGACAGGCTACAACCCAAGTATTAAGAG
CAGGATCTCTATCTCCAGAGATACCGGCAAAAATCAATTTTCACTCCAGT
TAAATTCTGTTACTGAAGAGGATACGGCCACTTACTACTGCGTACGAGGA
ATGGGTTACTTTGATGTCTGGGGCGCAGGAACATTGGTGACAGTCTCTAG
T
>112_19_LC Nucleotide sequence CDRL
SEQ ID NO: 123:
GACATCGTTATGACCCAGTCCCCCGCAACAATGGCTGTTTCCCCAGGTGA
GAGAGTGACCATCCATTGTAAGAGCAGCCAAAGCCTGTTTTATTCCGGAA
ATAATAAACACCTCCTGAGTTGGTACCAGCAAAAGCCCGGCCAGTCTCCT
AAACTGTTGATCTATTGGCCTTCAACTAGGGCCTCAGGCGTTCCAGATCG
CTTCAGTGGATCCGGATCCGGTACAGATTTCACTCTCACCATTAGTTCCC
TCCAAGCCGAGGATGTTGCAGATTACTTTTGTCAGCAGTTTATCAGCGCT
CCCCCTACATTCGGCGCCGGGACAAAGCTTGAAATAAAA
>112_20_HC Nucleotide sequence CDRH
SEQ ID NO: 124:
CAGATGCAACTGCAGGAGAGTGGCCCCGGACTTGTAAAGCCTTCTCAAAC
GCTGTTTCTCACTTGCAGTGTGTCCGGCTTCAGCATCACAACATCTGGAA
GCGGGTGGTCCTGGATCAGACAACCACGCGGGAAAACTTTAGAAGTGATG
GGACTGATTGCCTACAATGGAGATACCGGTTATAACCCTAGTATCAAATC
TCGCCTCAGTATAAGTCGGGATACAGGAAAAAACCAGTTTAGCCTTCAAT
TGAACAGTGTGACTGAAGAAGATACTGCTACCTACTACTGTGCGAGGGGG
GGAGGGTACTTTGACGTCTGGGGCGCTGGCACCTTAGTGACAGTCTCCTC
C
>112_20_LC Nucleotide sequence CDRL
SEQ ID NO: 125:
GATATCGTGATGACACAGAGCCCGGCTACCATGGCCGTTTCTCCAGGCGA
GCGGGTGACCATCCATTGTAAGAGTAGTCAGTCTTTGTTTTACTCCGGCA
ACAATAAATATTTGTTGAGTTGGTACCAGCAGAACCCAGGTCAGTCTCCG
CGCCTGCTGATCTACTGGGCTTCTACACGCGCTAGTGGAGTACCTGACAG
ATTCAGCGCATCCGGATCAGGCACCGATTTCACCTTGACTATCTCCAGCC
TCCAGGCCGAAGACGTCGCAGACTATTTCTGCCAGCAGTTTATCTCCGGC
CCCCCAACGTTCGGGGCCGGCACAAAGCTCGAGATCAAG
>112_21_HC Nucleotide sequence CDRH
SEQ ID NO: 126:
CAGATGCAGTTACAGGAGTCAGGACCGGGACTGGTTAAGCCTTCCCAGAC
CCTGTTTCTCACATGCAGCGTGTCTGGTTTTTCAATCACTACCAGCGGCT
CTGGGTGGAGCTGGATAAGGCAGCCAAGGGGAAAGACCCTGGAAGTTATG
GGAGGGTTTGCCTACAATGGAGACACGGCTTATAACCCGTCCATCACATC
CAGAATTTCCATCTCTAGGGACACAGGGAAGAATCAATTTTCTCTGCAGC
TCAATTCCGTGACTGAAGAGGATACCGCCACTTACTATTGTGCGAGAGGC
ATGGGCTACTTTGACGTCTGGGGGGCAGGCACACTGGTTACAGTCTCATC
T
>112_21_LC Nucleotide sequence CDRL
SEQ ID NO: 127:
GACATCGTCATGACCCAGTCCCCGGCCACAATGGCCGTCTCTCCAGGGGA
GAGAGCTACAATACACTGTAAGTCATCCCAAACCCTGTTTTACTCTGGGA
ATAAAAAACACCTCCTTACCTGGTATCAACAGAAGCCAGGCCAGTCACCC
AAAATGATTATTAATTGGGCATCCACGCGAGCAAGCGGCGTGCCTGATAG
GTTTAGCGGCTCAGGCAGTGGAACAGATTTTACTCTCACAATCAACTCTC
TGCAGGCCGAAGACGTGGCCGACTATTTTTGTCAGCAATTCATCCGCGGA
CCGCCAACGTTCGGCGCAGGAACAAAATTAGAAATCAAG
>112_22_LC Nucleotide sequence CDRL
SEQ ID NO: 128:
GACATACAGATGACACAATCCCCCTCATTCCTTTCTGCGTCCCTCGGGGA
CCGCGTCACAATTACCTGCAAGGCATCTCAGAACATTTATCGCAACTTGG
CCTGGTACCACCAGAAACCTGGCAAGGCCCCCAGGCTGCTTATCTACAGT
GCCTCTAATCTGGACTCTGGGGTCCCGTCTAGATTTAGCGGATTCGGCTC
AGGCACCGACTACACATTTATCATTGGCGCCGTAGAATCAGATGACGTGG
GCATCTACCACTGTCAGCAGTACGATAGTTATCCTCCTCTGACATTCGGT
GGCGGTACGAGGCTGGAAATTAAA
>112_23_LC Nucleotide sequence CDRL
SEQ ID NO: 129:
GACATTCAGATGACACAATCACCCTCCTTTTTATCAGCCAGCCTCGGAGA
TCGGGTGACAATTTCTTGTAAGGCTTCTCAGAATATTCAGCGTAACCTGG
CCTGGTACCACCAGAGGCCGGGCAAGGCCCCTAGACTGCTGATATATGCC
GCCAGTAGTCTGGACAGTGGGGTGCCTAGCCGTTTTTCAGGGTCAGGCTC
CGGTACAGATTATACTTTTACCATTTCTACCGTGGAGCCAGATGACGTAG
GGATTTATCACTGTCAGCAGTACGACGGGTACAGTCCCCTGACGTTCGGA
GGCGGAACCCGGTTGGAGATCAAG
>112_25_HC Nucleotide sequence CDRH
SEQ ID NO: 130:
CAGGTCCAATTGCAGGAATCTGGACCTGGACTGGTAAAACCAAGCCAAGC
CCTGTCACTGACATGTGCGGTATCTGGATTTTCCATTACTACCACCTACT
ATGGATGGCACTGGTTTAGGCAGCCGAAAGGGAAGTCTCCAGAATGGATG
GGTGTGATCGCTTATAACGGGGACACGGGGTACAATCCCTCTCTGCAGTC
ACGAATCAGTATTTCCAGAGAAGCTGGCAAGAATCAGTTCTTTCTGAAGC
TGCACAGCGTGACCGAAGAGGATACGGCGATGTATTACTGCGTGCTGCGG
GGCACTAGAATGGTCACGATCGACCCGCATTTTGATGTGTGGGGCGCAGG
AACGCTTGTTACCGTGTCTTCC
>112_25_LC Nucleotide sequence CDRL
SEQ ID NO: 131:
TGCGACGTGTTACTGACCCAGCCCGCTAGCGCTAGTGCATCTGTTGGGGA
TACCGTAAAAATTTCCTGCCGAGCCTCCCAAAGCGTAGGGGACTTTCTGT
CTTGGTACCAGCAGAAGGCGGGAAAGGCCCCCCAACTGTTGATTTATGCC
GCCACCCACTTGGCCTATGGCGTGTCCAGCAGATTCTCTGGCTCTGGCAG
TGGAACGGACTTCACCCTCACTATAAGTAGCTTGCAGCCTGAGGACTTTG
GAACATACTATTGTCTGCAGACTTATATCTCTCCACGAACCTTTGGTGCC
GGGACAAACCTCGAGATAAAA
Amino acid sequence- VH1-58-54+JH2
SEQ ID NO: 132:
QVQLQESGPGLVKPSQLSITCTVSGFSITTTNYWWHWICQPPGKSLEWLG
EISYDGNTKYNPSVKSRLSIARDTGKNQFSLQLTSVTTEDTATYYCNFQY
WGQGTLVTVSS
Amino acid sequence- VK1-140+Jκ2
SEQ ID NO: 133:
DIQMTQSPSSLSASLGDRVTIITCKASLAWYHQKPGKAPRLLIYNLDSGV
PSRFSGSGSGTDFTLTISAVEPEDVGVYYCNTFGGGTKLEIK
Amino acid sequence- VH1-151-54+JH2
SEQ ID NO: 134:
QVQLQESGPGLVKPSQTLSLCTCVTGGFSITTSSYYWSWIRQPPGKSPEW
MGVIDYDGDTAYSPSLKSRISISRDTGKQFSLQLRSVTEDTATYYCNFQY
WGQGTLVTVSS
Amino acid sequence- VL5-74+JL3/6/10
SEQ ID NO: 135:
QELVTQEPSLSPPGTAARLTCTLRSDLSVGGSYRIFWYQQKPGSPPRFLL
HYHTDSDKQLGSGPSRFSRSGSKDTSANAGVLRISGLQPEDEADYYCYVF
GGGGTKLTVL

What is claimed is:

1. A method of determining broadly neutralizing antibodies in a guinea pig model system, comprising:
immunizing a guinea pig with a cocktail of two or more viral strain antigens from a virus;
isolating antigen-positive IgG-expressing B-cells from the guinea pigs immunized to the viral strain antigens using IgG^{positive}/IgM^{negative} flow panel;
labeling a plurality of viral strain antigens with unique antigen barcodes;
providing a plurality of barcode-labeled viral strain antigens to a population of the isolated IgG-expressing B-cells to form a mixture;
allowing the plurality of barcode-labeled viral strains to bind to the population of the isolated IgG-expressing B-cells;
washing unbound viral strain antigens from the population of isolated IgG-expressing B-cells;
separating the isolated IgG-expressing B-cells into single cell emulsions;
introducing into each single cell emulsion a unique cell barcode-labeled bead;
preparing a single cell cDNA library from the single cell emulsions;
performing nested PCR for target enrichment on the cDNA library using an inner reverse primer SEQ ID NO: 13, 14, or 15, an outer reverse primer SEQ ID NO:9, 10, 11, or 12, and a forward primer SEQ ID NO: 16 or 17;
isolating B-cell receptor (BCR) genes from the cDNA library in the single cell emulsions;
sequencing paired heavy and light chains of the BCR;
performing PCR amplification reactions to produce a plurality of amplicons, wherein the amplicons comprise: the cell barcode and a nucleic acid sequence that identifies a particular viral strain antigen, wherein each amplicon comprises a unique molecular identifier (UMI);
sequencing the plurality of amplicons;
removing a sequence lacking the cell barcode, the UMI, or the nucleic acid sequence that identifies the particular viral strain antigens;
aligning the antibody sequence to a reference library of immunoglobulin V, D, J and C sequences;
constructing a UMI count matrix comprising the cell barcode, the nucleic acid sequence that identifies the particular viral strain antigens, and the antibody sequence;
determining a LIBRA-seq score, wherein a low score means no predicted binding to antigens and a high score means high predicted binding to antigens;
determining the nucleic acid sequence that identifies the particular viral strain antigens; and
determining broadly neutralizing antibodies binding more than two viral strain antigens.

2. The method of claim 1, wherein the barcode-labeled viral strain antigens are labeled with a barcode comprising a DNA sequence or an RNA sequence.

3. The method of claim 1, wherein the cell barcode-labeled beads are labeled with a barcode comprising a DNA sequence or an RNA sequence.

4. The method of claim 1, wherein the antibody sequence comprises an immunoglobulin heavy chain (VDJ) sequence, or an immunoglobulin light chain (VJ) sequence.

5. The method of claim 1, wherein the barcode-labeled viral strain antigens comprise an antigen from a pathogen or an animal.

6. The method of claim 5, wherein the viral strain antigens are not purified.

7. The method of claim 5, wherein the pathogen is a virus.

8. The method of claim 7, wherein the viral strain antigens comprise HIV-1 envelope protein (Envs)-BG505 T332N, 97ZA012.29, 286.36, 5768.04, KNH1209.18, DU172.17, HT593.1, MB539.2B7, a CD4 binding site-specific antigen (RSC3), and/or a negative control antigen (CA/09 H1).

9. The method of claim 7, wherein the viral strain antigens from a virus comprises an antigen from human immunodeficiency virus (HIV), an antigen from influenza virus, or an antigen from respiratory syncytial virus (RSV).

10. The method of claim 9, wherein the viral strain antigen from HIV comprises HIV-1 Env.

11. The method of claim 9, wherein the viral strain antigen from influenza virus comprises hemagglutinin (HA).

12. The method of claim 9, wherein the viral strain antigen from RSV comprises an RSV F protein.

13. The method of claim 1, wherein the plurality of viral strain antigens comprises a panel of epitope knock-outs.

14. The method of claim 1, wherein the plurality of viral strain antigens comprises a panel of viral antigen variants or mutations for epitope mapping.

15. The method of claim 1, further comprising determining a length of a complementarity-determining region (CDR) of the antibody specifically binding to the viral strain antigen.

16. The method of claim 1, further comprising determining a motif of a CDR of the antibody specifically binding to the viral strain antigen.

17. The method of claim 16, wherein the CDR is selected from the group consisting of CDRH3 (SEQ ID NOS: 18-47) and CDRL3 (SEQ ID NOS: 50-79).

18. A method of developing a vaccine composition in a guinea pig model system, comprising:
immunizing a guinea pig with a cocktail of two or more viral strain antigens from a virus;
isolating antigen-positive IgG-expressing B-cells from the guinea pigs immunized to the viral strain antigens using IgG positive/IgM negative flow panel;
labeling a plurality of viral strain antigens with unique antigen barcodes;
providing a plurality of barcode-labeled viral strain antigens to a population of the isolated IgG-expressing B-cells to form a mixture;
allowing the plurality of barcode-labeled viral strains to bind to the population of the isolated IgG-expressing B-cells;
washing unbound viral strain antigens from the population of isolated IgG-expressing B-cells;
separating the isolated IgG-expressing B-cells into single cell emulsions;
introducing into each single cell emulsion a unique cell barcode-labeled bead;
preparing a single cell cDNA library from the single cell emulsions;
performing nested PCR for target enrichment on the cDNA library using an inner reverse primer SEQ ID NO: 13, 14, or 15, an outer reverse primer SEQ ID NO:9, 10, 11, or 12, and a forward primer SEQ ID NO: 16 or 17;
isolating B-cell receptor (BCR) genes from the cDNA library in the single cell emulsions;
sequencing paired heavy and light chains of the BCR;
performing PCR amplification reactions to produce a plurality of amplicons, wherein the amplicons comprise: the cell barcode and a nucleic acid sequence that identifies a particular viral strain antigen, wherein each amplicon comprises a unique molecular identifier (UMI);
sequencing the plurality of amplicons;
removing a sequence lacking the cell barcode, the UMI, or the nucleic acid sequence that identifies the particular viral strain antigens;
aligning an antibody sequence to a reference library of immunoglobulin V, D, J and C sequences;
constructing a UMI count matrix comprising the cell barcode, the nucleic acid sequence that identifies the particular viral strain antigens, and the antibody sequence;
determining a LIBRA-seq score, wherein a low score means no predicted binding to antigens and a high score means high predicted binding to antigens;
determining the nucleic acid sequence that identifies the particular viral strain antigens;
determining broadly neutralizing antibodies binding more than two viral strain antigens; and
formulating a vaccine composition comprising the particular viral strain antigens that specifically bind to broadly neutralizing antibodies, wherein the particular viral strain antigens are capable of eliciting an immune response in the guinea pig.

19. The method of claim 18, further comprises:
detecting paired heavy and light chain gene among IgG expressing B-cells from multiple guinea pigs administered with the same vaccine composition, thereby identifying vaccine composition public clonotypes.

20. The method of claim 18, wherein the vaccine composition comprises an adjuvant to enhance immune response.
